(19) United States
(12) Patent Application Publication
(10) Pub. No.: US 2025/0345442 A1
(43) Pub. Date: Nov. 13, 2025

(54) PROTEIN AND PEPTIDE DELIVERY SYSTEMS AND METHODS FOR MAKING AND USING THEM

(71) Applicant: San Diego State University (SDSU) Foundation, dba San Diego State University Research Foundation, San Diego, CA (US)

(72) Inventors: Nicholas SHIKUMA, San Diego, CA (US); Kyle MALTER, San Diego, CA (US)

(21) Appl. No.: 18/868,945

(22) PCT Filed: Jun. 1, 2023
(86) PCT No.: PCT/US2023/024108

§ 371 Date: Nov. 25, 2024

Related U.S. Application Data
(60) Provisional application No. 63/347,873, filed on Jun. 1, 2022.

(51) Int. Cl. A61K 47/64; C07K 14/195; C12N 9/20

(52) U.S. Cl. CPC A61K 47/645; A61K 47/6415; C07K 14/195; C12N 9/20; C12Y 301/01003; C12Y 301/01008

(57) ABSTRACT

Provided are compositions, kits, and methods for delivering a proteinaceous cargo, or a protein or a peptide, or a drug or a marker, to or into a cell or to an individual in need thereof. In alternative embodiments, products of manufacture as provided herein comprise: (a) a recombinant bacterial Contractile Injection System (CIS) or a Metamorphosis Associated Contractile structure (MAC) formed or configured to comprise a tube having an inner core, (b) a Metamorphosis-Inducing Factor 1 (Mif1) protein positioned in the inner core of the tube of the CIS or MAC, (c) a chaperone 605 protein non-covalently associated with the Mif1 protein positioned in the inner core of the tube of the CIS or MAC, and (d) a proteinaceous cargo, or a heterologous protein or peptide, or compound, non-covalently associated or covalently associated or linked to the Mif1.

### RELATED APPLICATIONS

This Patent Convention Treaty (PCT) International Application claims the benefit of priority under 35 U.S.C. § 119(e) of U.S. Provisional Patent Application Ser. No. (USSN) 63/347,873, filed Jun. 1, 2022. The aforementioned application is expressly incorporated herein by reference in its entirety and for all purposes. All publications, patents, patent applications cited herein are hereby expressly incorporated by reference for all purposes.

### STATEMENT AS TO FEDERALLY SPONSORED RESEARCH

This invention was made with government support under Department of Defense, Office of Naval Research (ONR) grant nos. N00014-17-1-2677; and N00014-20-1-2120; and, NSF grant no. 1942251. The government has certain rights in the invention.

### TECHNICAL FIELD

This invention generally relates to microbiology and bioengineering. In alternative embodiments, provided are chimeric products of manufacture and methods for delivering a proteinaceous cargo, a polypeptide or peptide, or a compound to or into a cell, for example, a eukaryotic cell such as a mammalian or a human cell, or to a plant cell, or to an individual in need thereof. In alternative embodiments, products of manufacture as provided herein comprise: (a) a recombinant bacterial Contractile Injection System (CIS) or a Metamorphosis Associated Contractile structure (MAC) formed or configured to comprise a tube having an inner core, (b) a Metamorphosis-Inducing Factor 1 (Mif1) protein positioned in the inner core of the tube of the CIS or MAC, (c) a chaperone 605 protein non-covalently associated with the Mif1 protein positioned in the inner core of the tube of the CIS or MAC, and (d) a proteinaceous cargo, or a heterologous protein or peptide, or compound, non-covalently associated or covalently associated or linked to the Mif1.

### BACKGROUND

Many bacteria interact with target organisms using syringe-like structures called Contractile Injection Systems (CIS). CIS structurally resemble headless bacteriophages and share evolutionarily related proteins such as the tail tube, sheath, and baseplate complex. Recent evidence shows that CIS are specialized to puncture membranes and often deliver effectors to target-cells. In many cases, CIS mediate trans-kingdom interactions between bacteria and eukaryotes, however the effectors delivered to target cells and their mode of action are often unknown.

A CIS mediating the beneficial relationship between the gram-negative bacterium Pseudoalteromonas luteoviolacea and marine tubeworm Hydroides elegans was recently characterized (Shikuma et al., 2014, 2016); and this CIS was named “Metamorphosis Associated Contractile structure” (MACs), because they stimulate the metamorphosis of Hydroides (Shikuma et al., 2014). While MACs provide an example of CIS-eukaryote interactions, the range of hosts targeted by CIS like MACs as well as the identity and mode of action of effectors that mediate these interactions remain poorly understood.

### SUMMARY

In alternative embodiments, provided are chimeric products of manufacture for delivering a proteinaceous cargo, or a heterologous protein or peptide, or a compound, into a cell, comprising:
(a) a recombinant bacterial Contractile Injection System (CIS) or a Metamorphosis Associated Contractile structure (MAC) formed or configured to comprise a tube having an inner core,
(b) a Metamorphosis-Inducing Factor 1 (Mif1) protein positioned in the inner core of the tube of the CIS or MAC,
(c) a chaperone 605 protein non-covalently associated with or covalently associated with or linked to the Mif1 protein positioned in the inner core of the tube of the CIS or MAC, and
(d) a proteinaceous cargo, or a heterologous protein or peptide, or compound, non-covalently associated or covalently associated or linked to the Mif1,
wherein optionally the proteinaceous cargo, the heterologous protein or peptide, or drug is chemically linked or electrostatically linked to the Mif1,
and optionally the compound is or comprises a small molecule, a lipid, a saccharide, a nucleic acid, a drug or a marker, optionally a detectable marker or a detectable moiety,
and optionally the proteinaceous cargo, the heterologous protein or peptide has enzymatic activity, optionally a lipase activity,
and optionally the proteinaceous cargo, the heterologous protein or peptide has binding activity, optionally heterologous protein or peptide comprises an antibody or antigen binding fragment,
and optionally the Mif1 protein is encoded by a nucleic acid sequence having at least about 85%, 86%, 87%, 88%, 89%, 90%, 91%, 92%, 93%, 94%, 95%, 96%, 97%, 98% or 100% sequence identity to SEQ ID NO:1, or between about 80% to 100% sequence identity to SEQ ID NO:1,
and optionally the Mif1 protein comprises a sequence having at least about 85%, 86%, 87%, 88%, 89%, 90%, 91%, 92%, 93%, 94%, 95%, 96%, 97%, 98% or 100% sequence identity to SEQ ID NO:2, or between about 80% to 100% sequence identity to SEQ ID NO:2,
and optionally CIS or MAC proteins are encoded by a nucleic acid sequence having at least about 85%, 86%, 87%, 88%, 89%, 90%, 91%, 92%, 93%, 94%, 95%, 96%, 97%, 98% or 100% sequence identity to SEQ ID NO:5, or between about 80% to 100% sequence identity to SEQ ID NO:5,
and optionally the chaperone 605 protein is encoded by a nucleic acid sequence having at least about 85%, 86%, 87%, 88%, 89%, 90%, 91%, 92%, 93%, 94%, 95%, 96%, 97%, 98% or 100% sequence identity to SEQ ID NO:3, or between about 80% to 100% sequence identity to SEQ ID NO:3,
and optionally the chaperone 605 protein comprises a sequence having at least about 85%, 86%, 87%, 88%, 89%, 90%, 91%, 92%, 93%, 94%, 95%, 96%, 97%, 98% or 100% sequence identity to SEQ ID NO:4, or between about 80% to 100% sequence identity to SEQ ID NO:4.

In alternative embodiments, provided are liposomes or lipid-comprising nanoparticle comprising, or incorporating or expressing on its outer surface, a chimeric product of manufacture as provided herein.

In alternative embodiments, provided are protoplasts or a spheroplasts comprising, or incorporating or expressing on its outer surface, a chimeric product of manufacture as provided herein.

In alternative embodiments, provided are cells comprising, or expressing on its extracellular surface, a chimeric product of manufacture as provided herein, wherein optionally the cell is a microbial cell or a eukaryotic cell, and optionally the microbial cell is a bacterial cell or a yeast cell, or a human cell.

In alternative embodiments, provided are methods for delivering a proteinaceous cargo, or a protein or a peptide, or a compound, to a cell, optionally to a eukaryotic, mammalian or human cell, or to a plant cell, or to an individual in need thereof, comprising contacting the cell with:
a chimeric product of manufacture as provided herein,
a liposome or lipid-comprising nanoparticle as provided herein,
a protoplast or a spheroplast as provided herein, or
a cell as provided herein,
under conditions wherein the proteinaceous cargo, or the protein or peptide, or the compound, is delivered into the cell.

In alternative embodiments of methods as provided herein:
the proteinaceous cargo, or the protein or peptide, comprises or is an antibody or an enzyme or an active biological agent;
the contacting of the formulation or composition with the cell eukaryotic cell is in vitro, ex vivo, or in vivo; and/or
the eukaryotic cell is a mammalian, human or an animal cell.

In alternative embodiments, provided are pharmaceutical compositions or formulations comprising:
a chimeric product of manufacture as provided herein,
a liposome or lipid-comprising nanoparticle as provided herein,
a protoplast or a spheroplast as provided herein, or
a cell as provided herein.

In alternative embodiments, provided are kits comprising:
a chimeric product of manufacture as provided herein,
a liposome or lipid-comprising nanoparticle as provided herein,
a protoplast or a spheroplast as provided herein, or
a cell as provided herein,
wherein optionally the kit further comprises instructions for practicing a method as provided herein.

In alternative embodiments, provided are uses of:
a chimeric product of manufacture as provided herein,
a liposome or lipid-comprising nanoparticle as provided herein,
a protoplast or a spheroplast as provided herein, or
a cell as provided herein,
for delivery of a proteinaceous cargo, a protein or peptide, or a compound, into a cell,
wherein optionally the delivery of the proteinaceous cargo, or the protein or peptide, or compound, into the cell is in vitro, ex vivo, or in vivo.

In alternative embodiments, provided are products of manufacture for use in delivering a proteinaceous cargo, a protein or peptide, or a compound, into a cell, wherein the product of manufacture is or comprises:
a chimeric product of manufacture as provided herein,
a liposome or lipid-comprising nanoparticle as provided herein,
a protoplast or a spheroplast as provided herein, or
a cell as provided herein.

The details of one or more embodiments as provided herein are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### BRIEF DESCRIPTION OF THE DRAWINGS

The drawings set forth herein are illustrative of embodiments as provided herein and are not meant to limit the scope of the invention as encompassed by the claims.

FIG. 1A-C illustrates Mif1 alpha fold prediction;

FIG. 1A schematically illustrates ALPHAFOLD2™ prediction of the effector protein Mif1;

FIG. 1B graphically illustrates the predicted IDDT local superposition-free score for each residue 1-943;

FIG. 1C graphically illustrates predicted alignment error of predicted residues vs scored residues;

FIG. 1D graphically illustrates sequence coverage of predicted residues; and

FIG. 1E-F graphically illustrate images of negative staining transmission electron microscopy of purified Mif1.

FIG. 2A-B illustrate domains of Mif1 required for Hydroides metamorphosis:

FIG. 2A graphically illustrates data where two hundred amino acid residues were systematically removed from Mif1 in order to determine their role in Mif1 effector loading; and

FIG. 2B graphically illustrates data from metamorphosis assays of extracted MACs complexes with the various mutants (including Mif1 knockouts, as indicated) were tested and assessed for their ability to induce metamorphosis.

FIG. 3A-C illustrate that Mif1 amino acid residues are required for binding with the MACs loading protein 1 (Mlp1):

FIG. 3A schematically illustrates the design of Mif1 protein fragments to be expressed in E. coli with the full Mlp1 protein for recombinant protein analysis, and identifies the Mif1 amino acid residues are required for binding with the MACs loading protein 1;

FIG. 3B illustrates a Western blot showing the presence of Mlp1 tagged with a S-tag. Ni²⁺ agarose pull-down using Mif1 or Mif1 fragments was washed of unbound protein and the resultant preparation was blotted for the presence of Mlp11, total lysate was used for comparison of pull-down protein versus total expressed protein; and

FIG. 3C illustrates a Western blot showing data from where a reciprocal S-tag was also used as bait and the Mif1 or Mif1 fragments were blotted by 6×His tag antibody.

FIG. 4A-B illustrate images of the N- and C-termini of Mif1 are toxic when overexpressed in E. coli:

FIG. 4A illustrates E. coli expressing recombinant mif1, mif1 fragments A-E, JF50_0605 or gfp genes from an IPTG inducible promoter in a pET15b vector in the presence or absence of 0.1 mM IPTG. Bacteria were grown overnight and then spotted by 1/5 serial dilutions starting at OD 1.0; and

FIG. 4B illustrates E. coli expressing recombinant mif1 fragments A1-3, C1-3 from an IPTG inducible promoter in a pET15b vector in the presence or absence of 0.1 mM IPTG; bacteria were grown overnight and then spotted by 1/5 serial dilutions starting at OD 1.0.

FIG. 5A-D illustrate that Mif1 binds membrane lipids and possesses lipase activity:

FIG. 5A illustrates images of lipid spotted membrane with various membrane lipids;

FIG. 5B illustrates images of Far western using purified Mif1 protein and Mif1 specific antibody shows binding to both PI3P and PA; and

FIG. 5C illustrates images of a lipid cleavage assay with purified Mif1 protein or chaperone (12605) protein, incubated for 1 hour with decanoic acid-PNPP substrate, cleavage and PnPP (4-nitrophenyl phosphate) release occurs if acyl-ester linkage is hydrolyzed.

FIG. 6A-E illustrate that Mif1 possesses lipase activity:

FIG. 6A graphically illustrates data from a lipid cleavage assay with purified Mif1 protein or chaperone (12605) protein, or a GFP control protein incubated with Tween-20 in the presence of Ca²⁺;

FIG. 6B graphically illustrates data from a assay where purified proteins were incubated for 1 hour with decanoic acid-PNPP substrate, and cleavage and PnPP (4-nitrophenyl phosphate) release occurs if acyl-ester linkage is hydrolyzed;

FIG. 6C graphically illustrates data from a PLD specific lipid cleavage assay with phosphatidylcholine substrate to assess enzymatic cleavage site of lipases by presence of choline release;

FIG. 6D graphically illustrates data from a Phospholipase A2 specific cleavage assay with Mif1, Buffer, or a control protein GH1; and

FIG. 6E graphically illustrates data from a Phospholipase C specific cleavage assay with Buffer, Mif1 or 605 control protein.

FIG. 7A-B illustrates Mif1 fragment analysis for lipase activity:

FIG. 7A graphically illustrates a Pnpp-decanoic acid lipase assay with purified proteins from BL21 plysE E. coli; the average of 4 technical replicates is shown; and

FIG. 7B graphically illustrates a tween-20 esterase assay of individually purified Mif1 fragments; the average of 3 technical replicates is shown.

FIG. 8 schematically illustrates a table of psiBLAST hits from full length Mif1 which identified full length Mif1 domain from psiBLAST hits (DUF4157).

FIG. 9A-B illustrates related strains of bacteria with similar Mif1 homologues stimulate Hydroides metamorphosis:

FIG. 9A schematically illustrates a maximum likelihood tree showing the relatedness of Mif1 homologs in marine bacteria; and

FIG. 9B graphically illustrates data from a metamorphosis assay of marine bacteria possessing a Mif1 homolog related to P. Luteo.

FIG. 10 schematically illustrates the alignment of Mif1 and E. coli hemolysin E pore forming toxin via PHYRE2™ (Protein Homology/AnalogY Recognition Engine) (Creative Commons Attribution-2.0); SEQ ID NO:6 illustrates the query sequence, and SEQ ID NO: 7 illustrates the template sequence.

FIG. 11 graphically illustrates data showing metamorphosis of larvae is not affected by lipids produced during lipase assay; metamorphosis assay of Hydroides with lipids isolated after incubation with purified recombinant protein.

Like reference symbols in the various drawings indicate like elements.

Reference will now be made in detail to various exemplary embodiments of the invention, examples of which are illustrated in the accompanying drawings. The following detailed description is provided to give the reader a better understanding of certain details of aspects and embodiments of the invention, and should not be interpreted as a limitation on the scope of the invention.

### DETAILED DESCRIPTION

In alternative embodiments, provided are chimeric products of manufacture and methods for delivering a proteinaceous cargo, a protein or a peptide, or compound such as a drug or a marker, to a cell such as a eukaryotic cell such as a human cell, or to an individual in need thereof.

In alternative embodiments, methods as provided herein comprise use of chimeric products of manufacture as provided herein to deliver a proteinaceous cargo, a protein or a peptide, or compound such as a drug or a marker, to a cell such as a eukaryotic cell such as a human cell, or to an individual in need thereof.

### Generating and Manipulating Nucleic Acids

In alternative embodiments, nucleic acids used to generate protein components of products of manufacture as provided herein, including (a) a recombinant bacterial Contractile Injection System (CIS) or a Metamorphosis Associated Contractile structure (MAC) formed or configured to comprise a tube having an inner core, (b) a Metamorphosis-Inducing Factor 1 (Mif1) protein positioned in the inner core of the tube of the CIS or MAC, (c) a chaperone 605 protein non-covalently associated with the Mif1 protein positioned in the inner core of the tube of the CIS or MAC, and (d) a proteinaceous cargo, or a heterologous protein or peptide, or compound, non-covalently associated or covalently associated or linked to the Mif1. In alternative embodiments, nucleic acids used to practice methods as provided herein.

In alternative embodiments, nucleic acids used to practice embodiments as provided herein, for example, encoding components of products of manufacture as provided herein, for example, comprising nucleic acids encoding MACs or CIS, Mif1, chaperone 605 protein and/or payload, are isolated and/or manipulated by, or inserted into bacteria and expressed, for example, by cloning and expression of cDNA libraries, amplification of message or genomic DNA by PCR, and the like. The nucleic acids and genes used to practice this invention, including DNA, RNA, iRNA, antisense nucleic acid, cDNA, genomic DNA, vectors, viruses or hybrids thereof, can be isolated from a variety of sources, genetically engineered, amplified, and/or expressed/generated recombinantly. Recombinant polypeptides generated from these nucleic acids can be individually isolated or cloned and tested for a desired activity. Any recombinant expression system or gene therapy delivery vehicle can be used, including for example, viral (for example, AAV constructs or hybrids) bacterial, fungal, mammalian, yeast, insect or plant cell expression systems or expression vehicles.

Alternatively, nucleic acids used to practice methods as provided herein, or to make products of manufacture, compositions or recombinant bacteria as provided herein, can be synthesized in vitro by well-known chemical synthesis techniques, as described in, for example, Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids as provided herein, or to make compositions or recombinant bacteria as provided herein, such as, for example, subcloning, labeling probes (for example, random-primer labeling using Klenow polymerase, nick translation, amplification), sequencing, hybridization and the like are well described in the scientific and patent literature, see, for example, Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice methods as provided herein, or to make compositions or recombinant bacteria as provided herein, is to clone from operons or genomic samples, and, if desired, screen and re-clone inserts isolated or amplified from, for example, genomic clones or cDNA clones. Sources of nucleic acid used in the methods of the invention include genomic or cDNA libraries contained in, for example, mammalian artificial chromosomes (MACs), see, for example, U.S. Pat. Nos. 5,721,118; 6,025,155; human artificial chromosomes, see, for example, Rosenfeld (1997) Nat. Genet. 15:333-335; yeast artificial chromosomes (YAC); bacterial artificial chromosomes (BAC); P1 artificial chromosomes, see, for example, Woon (1998) Genomics 50:306-316; P1-derived vectors (PACs), see, for example, Kern (1997) Biotechniques 23:120-124; cosmids, recombinant viruses, phages or plasmids.

In alternative embodiments, a heterologous peptide or polypeptide joined or fused to a protein made by a method or a recombinant bacteria as provided herein can be an N-terminal identification peptide which imparts a desired characteristic, such as fluorescent detection, increased stability and/or simplified purification. Peptides and polypeptides made by a method or a recombinant bacteria as provided herein can also be synthesized and expressed as fusion proteins with one or more additional domains linked thereto for, for example, producing a more immunogenic peptide, to more readily isolate a recombinantly synthesized peptide, to identify and isolate antibodies and antibody-expressing B cells, and the like. Detection and purification facilitating domains include, for example, metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle WA). The inclusion of a cleavable linker sequences such as Factor Xa or enterokinase (Invitrogen, San Diego CA) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see for example, Williams (1995) Biochemistry 34:1787-1797; Dobeli (1998) Protein Expr. Purif. 12:404-414). The histidine residues facilitate detection and purification while the enterokinase cleavage site provides a means for purifying the epitope from the remainder of the fusion protein. Technology pertaining to vectors encoding fusion proteins and application of fusion proteins are well described in the scientific and patent literature, see for example, Kroll (1993) DNA Cell. Biol., 12:441-53.

Nucleic acids or nucleic acid sequences used to practice embodiments as provided herein can be an oligonucleotide, nucleotide, polynucleotide, or to a fragment of any of these, to DNA or RNA of genomic or synthetic origin which may be single-stranded or double-stranded and may represent a sense or antisense strand, to peptide nucleic acid (PNA), or to any DNA-like or RNA-like material, natural or synthetic in origin. Compounds use to practice this invention include “nucleic acids” or “nucleic acid sequences” including oligonucleotide, nucleotide, polynucleotide, or any fragment of any of these; and include DNA or RNA (for example, mRNA, rRNA, RNA, iRNA) of genomic or synthetic origin which may be single-stranded or double-stranded; and can be a sense or antisense strand, or a peptide nucleic acid (PNA), or any DNA-like or RNA-like material, natural or synthetic in origin, including, for example, iRNA, ribonucleoproteins (for example, for example, double stranded iRNAs, for example, iRNPs). Nucleic acids or nucleic acid sequences used to practice embodiments as provided herein include nucleic acids or oligonucleotides containing known analogues of natural nucleotides. Nucleic acids or nucleic acid sequences used to practice embodiments as provided herein include nucleic-acid-like structures with synthetic backbones, see for example, Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156. Nucleic acids or nucleic acid sequences used to practice embodiments as provided herein include “oligonucleotides” including a single stranded polydeoxynucleotide or two complementary polydeoxynucleotide strands that may be chemically synthesized. Compounds use to practice this invention include synthetic oligonucleotides having no 5′ phosphate, and thus will not ligate to another oligonucleotide without adding a phosphate with an ATP in the presence of a kinase. A synthetic oligonucleotide can ligate to a fragment that has not been dephosphorylated.

In alternative aspects, methods and recombinant bacteria as provided herein comprise use of “expression cassettes” comprising a nucleotide sequences capable of affecting expression of the nucleic acid, for example, a structural gene or a transcript (for example, encoding a Contractile Injection System (CIS)) in a host compatible with such sequences. Expression cassettes can include at least a promoter operably linked with the polypeptide coding sequence or inhibitory sequence; and, in one aspect, with other sequences, for example, transcription termination signals. Additional factors necessary or helpful in effecting expression may also be used, for example, enhancers.

In alternative aspects, expression cassettes used to practice embodiments as provided herein also include plasmids, expression vectors, recombinant viruses, any form of recombinant “naked DNA” vector, and the like. In alternative aspects, a “vector” used to practice embodiments as provided herein can comprise a nucleic acid that can infect, transfect, transiently or permanently transduce a cell. In alternative aspects, a vector used to practice embodiments as provided herein can be a naked nucleic acid, or a nucleic acid complexed with protein or lipid. In alternative aspects, vectors used to practice embodiments as provided herein can comprise viral or bacterial nucleic acids and/or proteins, and/or membranes (for example, a cell membrane, a viral lipid envelope, etc.). In alternative aspects, vectors used to practice embodiments as provided herein can include, but are not limited to replicons (for example, RNA replicons, bacteriophages) to which fragments of DNA may be attached and become replicated. Vectors thus include, but are not limited to RNA, autonomous self-replicating circular or linear DNA or RNA (for example, plasmids, viruses, and the like, see, for example, U.S. Pat. No. 5,217,879), and can include both the expression and non-expression plasmids. In alternative aspects, the vector used to practice embodiments as provided herein can be stably replicated by the cells during mitosis as an autonomous structure, or can be incorporated within the host's genome.

In alternative aspects, “promoters” used to practice this invention include all sequences capable of driving transcription of a coding sequence (for example, for a Contractile Injection System (CIS)) in a cell, for example, a bacterial cell. Thus, promoters used in the constructs of the invention include cis-acting transcriptional control elements and regulatory sequences that are involved in regulating or modulating the timing and/or rate of transcription of a gene. For example, a promoter used to practice this invention can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) transcription.

### Bacterial Contractile Injection System (CIS) or Metamorphosis Associated Contractile Structures (MAC)

In alternative embodiments, products of manufacture as provided herein comprise a Bacterial Contractile Injection System (CIS) or a Metamorphosis Associated Contractile structure (MAC), which are a toxin-delivery particle that evolved from a bacteriophage tail, as described for example in Geller, A. M., Pollin, I., Zlotkin, D. et al. The extracellular contractile injection system is enriched in environmental microbes and associates with numerous toxins. Nat Commun 12, 3743 (2021). In alternative embodiments, the CIS or MAC is homologous to a bacteria from which the CIS or MAC is isolated for use in a product of manufacture as provided herein, or the CIS or MAC is heterologous to a bacteria, and coding sequence

Bacterial CISs as provided herein can be extracellular CISs (eCISs) or type VI secretion systems (T6SSs), as described for example in Xu et al, Nature Microbiology volume 7, pgs 397-410 (2022). eCISs resemble headless phage particles that are assembled in the bacterial cytoplasm and then released into the medium upon cell lysis, and upon binding to a target cell via tail fibres, and eCISs contract and puncture the target's cell envelope. T6SSs remain intracellular and are anchored to the inner membrane, injecting payloads by a cell-cell contact-dependent mechanism.

In alternative embodiments, the CIS or MAC structure comprises a contractile sheath enveloping a rigid tube that is sharpened by a spike-shaped protein complex at its tip. The spike complex forms the centerpiece of a baseplate complex that terminates the sheath and the tube. The baseplate anchors the tail to the target cell membrane with the help of fibrous proteins emanating from it and triggers contraction of the sheath. The contracting sheath drives the tube with its spiky tip through the target cell membrane, thus resulting in injection of a payload through the tube.

In alternative embodiments, the protein subunits that comprise a CIS or MAC complex can encoded by an operon having a nucleic acid sequence having at least about 85%, 86%, 87%, 88%, 89%, 90%, 91%, 92%, 93%, 94%, 95%, 96%, 97%, 98% or 100% sequence identity to SEQ ID NO:5, or between about 80% to 100% sequence identity to SEQ ID NO:5.

### Metamorphosis-Inducing Factor 1 (Mif1) Proteins

In alternative embodiments, products of manufacture as provided herein comprise a Metamorphosis-Inducing Factor 1 (Mif1) protein positioned in the inner core of the tube of the CIS or MAC; and a proteinaceous cargo, or a heterologous protein or peptide, or compound, is non-covalently associated or covalently associated or linked to the Mif1.

In alternative embodiments, the Mif1 protein is encoded by a nucleic acid sequence having at least about 85%, 86%, 87%, 88%, 89%, 90%, 91%, 92%, 93%, 94%, 95%, 96%, 97%, 98% or 100% sequence identity to SEQ ID NO:1, or between about 80% to 100% sequence identity to SEQ ID NO:1, or optionally the Mif1 protein comprises a sequence having at least about 85%, 86%, 87%, 88%, 89%, 90%, 91%, 92%, 93%, 94%, 95%, 96%, 97%, 98% or 100% sequence identity to SEQ ID NO:2, or between about 80% to 100% sequence identity to SEQ ID NO:2.

### Chaperone 605 Proteins

In alternative embodiments, products of manufacture as provided herein comprise chaperone 605 proteins, which are associated with the Mif1 protein component of the product of manufacture as provided herein. A chaperone 605 protein can be non-covalently associated with or covalently associated with or linked to the Mif1 protein positioned in the inner core of the tube of the CIS or MAC.

In alternative embodiments, the chaperone 605 protein is encoded by a nucleic acid sequence having at least about 85%, 86%, 87%, 88%, 89%, 90%, 91%, 92%, 93%, 94%, 95%, 96%, 97%, 98% or 100% sequence identity to SEQ ID NO:3, or between about 80% to 100% sequence identity to SEQ ID NO:3, and/or the chaperone 605 protein comprises a sequence having at least about 85%, 86%, 87%, 88%, 89%, 90%, 91%, 92%, 93%, 94%, 95%, 96%, 97%, 98% or 100% sequence identity to SEQ ID NO:4, or between about 80% to 100% sequence identity to SEQ ID NO:4.

### Sequence Identity Determinations

In alternative embodiments, a sequence identity is calculated using a sequence comparison algorithm consisting of a BLAST version 2.2.2 algorithm where a filtering setting is set to blastall-p blastp-d “nr pataa”-F F, and all other options are set to default. In alternative embodiments, protein and/or nucleic acid sequence homologies are calculated using any of the variety of sequence comparison algorithms and programs known in the art. Such algorithms and programs include, but are by no means limited to, TBLASTN, BLASTP, FASTA, TFASTA and CLUSTALW (Pearson and Lipman, Proc. Natl. Acad. Sci. USA 85(8):2444-2448, 1988; Altschul et al., J. Mol. Biol. 215(3):403-410, 1990; Thompson et al., Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993).

In alternative embodiments, the sequence identity (homology) is calculated using BLAST and BLAST 2.0 algorithms, which are described in Altschul et al., Nuc. Acids Res. 25:3389-3402, 1977 and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3 and expectations (E) of 10 and the BLOSUM62 scoring matrix (see Henikoff & Henikoff, Proc. Natl. Acad. Sci. USA 89:10915, 1989) alignments (B) of 50, expectation (E) of 10, M=5, N=−4 and a comparison of both strands.

In alternative embodiments, protein and nucleic acid sequence homologies (sequence identity) are evaluated using the Basic Local Alignment Search Tool (“BLAST”) In particular, five specific BLAST programs are used to perform the following task: (1) BLASTP and BLAST3 compare an amino acid query sequence against a protein sequence database; (2) BLASTN compares a nucleotide query sequence against a nucleotide sequence database; (3) BLASTX compares the six-frame conceptual translation products of a query nucleotide sequence (both strands) against a protein sequence database; (4) TBLASTN compares a query protein sequence against a nucleotide sequence database translated in all six reading frames (both strands); and (5) TBLASTX compares the six-frame translations of a nucleotide query sequence against the six-frame translations of a nucleotide sequence database.

| SEQ ID NO: 1 (Mif1 protein) is: | |
|---|---|
| ATGCAACAACAAGAACAGGAGCAAGCTCCTACCTTTCAAAGTTATCCCAC | |
| GCGCACTGCGTTATCAGTGAGCAATGCATTAGACTCTGATTCTGTGAGTGT | |
| CGATCTACTCTCATCGGGCGTTGATGTTGCAAAGCGGTATGCCTCACAAGT | |
| GGTTTGGGATCATTTTAATGGCGATGCAACAGCTAAGCTGATTATCTCAAG | |
| TGTATTTAATTATGCGGTGACATCATTAAAGTCCTTGAACCCATGGGTGAC | |
| GGCAATCTCACAAGCACTTTTACTTTTGGCAAAAGTTCCGCCTGGTGTTGT | |
| TTCCGCCGTTTTATGGGCCATTGGAAAAATCTGGCTTTGGGCTGCAAATAA | |
| ATTTTATAACGGTGGTTGGATAGCCGCTGCGTGGGGAGATATTGATGAGC | |
| CATATATTTATCAATGGTTAAAAAAAGGCAGTGATGCACATGGGGCATTA | |
| CGGGCACTCGTGGATGATTTAAAAGCTTGGGTTAAGTATATTCAAGATAA | |
| GCTTGCCAGTAGCGTTGCACGTTTAATTGGTGTCTCGGATTCAAGTAGTGA | |
| AGATGAGCAAAGCGATGAGCAACAAACGGATCAAGATGCACAAACATCA | |
| CCTAATATAGTTGATAATAAGTTCATTTCGTTGGGGATAAACCAACCTGAA | |
| TTATTGGACTGGGAAGAGGGGGCAAGTAAGCCCAAGCGCGCTGGACTAC | |
| ATGGTACGGGTGTTGCCAAGGTCAATCTGCTTGGTCAGCAATTTGGTGGTG | |
| ATATAGATGTAAAATTACCATTTGGCAGTGGCTGGGAAGTGGCTGTATCT | |
| CCGGTTTTTGCAAGCCAACAAGGGGTGGGTTTTAGTGGTTATATTGAAGC | |
| GCATCAAGTATTGGGCGATGAGCTGACCATTAATGAAGAAGGCCTTGCTC | |
| GTTTTAAAGCAAGCATTGTTGGGCTAAACATTGCGAACAAGCGGGTTTAC | |
| TCCGATTTAATGTCACTTGATTACAATAAGCAGCAAAAAGAAGTTCATTTT | |
| GCAGGTGATGCACATGTGCCGCTTTGGGATAAAAAGAAACTGGATGGTGA | |
| GTTTGATTTAAAATTGGATACTGCAGGTAAATTCAAATCAGGCAGGACCA | |
| AAATCTCTTCTAAAGATACATTCGAAGTAATCCCCAAGTTTTTGTCTATCA | |
| GCAACCCGAGTGGTGAAGTAGAAGTTAAAGAGGATGCTTCACCAGAGTTT | |
| GAGGTGAGTACTGACGCTGCGCTTTACGGCTTACCCGCTGGTGTGAAAGC | |
| ATCAATTACGCAAGCAAAAGTGATGTATCACGATGAGCAATTAGGCGGTG | |
| AGGTTGAGCAGGCTGATGTGGAGATCCCAATTAGCAAACACACTACAATG | |
| ACGCTGGCCATGACGAAGGCTAAGTTTACAAAAGATGCAATTGAAGCTGA | |
| AAATGCTTTTATGGATCTCGATCATGATAATGCCAAAGTGCTTAATGAAAC | |
| GGCCATTGTCGAGTCAGACTTTTTCTCTGGAAACTTCGATTTTGGAAAAGT | |
| GTTTGAGCTTAAGCAGTTGAAAGTCCACCAAGGGTTAAACGCGCTTAAAT | |
| TTGCTGGTGGTAAATTCTCCTACGAGAAGGATAGCAACAATGGTTTACAG | |
| TTACTTAGAGCACGTATTTTAGGCCTTGAGGCTTACTATAATAGAAAAGAT | |
| CGTGAAGGTGGGATCACTGGAGAATGGCACAAAGGGATTGATTTTCCGGC | |
| TTTCTCTTTGGACTTTCCCGTTGCGGCTGGTGTTGCAGGAGTTGGAGTTGA | |
| TATTACCGGGGGGTTTGAGTTTGGTGCGAGCATCGGCGCTAAGCTTAAAA | |
| ATGAAGAGCAGCATAACACAGAGACACAAATGCTTTTCTCTGTGAATGGT | |
| GCAGCTTCGGCCAGTGCCAAAGCTAAAGCGAGAATTGAGGCTGGTGCTTT | |
| TGTGGGGGTCCCTTATCTTGCAAAGGTTCAAGGTGGTGTATTTGGTGAGAT | |
| CCGAGGTGGTGTTGAAGGTAAGGTTGAAACAGAAGGGCGTTTGAAATACA | |
| CGCGTGGCAAAGGCGGTGATAGCTTTGGTCAATTTGCCATTGATAGTGATT | |
| ATCCAATGGAGGCGAGCTTTAGCCTAACCGGCTCATTAGAAGCGGAAGTA | |
| GGTGCATCGATAAAAGCCAAAGTACTGACATTTGAAAAAGAAATTGCGTC | |
| GGTATCTATTGGTGATTGGACACTGGGTGAGTATACGCTTAATGGTAAAA | |
| TCAAAAAAGATCCTAATGGGAAAGGGTATATCGTTGAGCGCTCTAAAGGG | |
| GAGTTTACCGAAGGCAAACCAAAACCTGCCGAGGTAACTAAGGAGGCAC | |
| TCCCAGTAGATAAGTGGGTTAATCAGTTAGAAAAAGATCATACTCATATT | |
| GAATACACCGAAGATGAAGCCAAAAGAAAGTTACCTGAGGTTGCACGTA | |
| AAACGGTATATAGTCACCTGAGTCCAATGCAAAAAGAGGATGTAAGTGAG | |
| CGATATACTCATTTGCTCAAGTTGATATCGCAGCAAAATAAGTTCACTGAA | |
| ATTTATCGAGAAACCAAAGCTGATAGGGATAAGTTACCGACCAGTGGCAC | |
| TTATATTTGGACCTCTAAAGTTTGGAATGAAGCGGTACAGCGCAAGAGTT | |
| TCTGGATTTTTGAAATGAGTAAGAGTAAAGCAAAAGTGGCGGATAAGCTG | |
| GATAAATATCACAAAACGCACTCTATTTTAGAGCGGAGACTGTTACTTGC | |
| AAAGCTAGAAGAGATAGCCTCGGACTATCGCAAAAACCGCTCGAAAAAC | |
| GCAGAAAATAAAGAAGCTGCCGGGGAGTTTTTAGAGAGTATTGAAAAAG | |
| AAAGGCTCATTTTAATGTAA | |
| SEQ ID NO: 2 (Mif1 protein) is: | |
| MQQQEQEQAPTFQSYPTRTALSVSNALDSDSVSVDLLSSGVDVAKRYASQVV | |
| WDHFNGDATAKLIISSVFNYAVTSLKSLNPWVTAISQALLLLAKVPPGVVSAV | |
| LWAIGKIWLWAANKFYNGGWIAAAWGDIDEPYIYQWLKKGSDAHGALRAL | |
| VDDLKAWVKYIQDKLASSVARLIGVSDSSSEDEQSDEQQTDQDAQTSPNIVD | |
| NKFISLGINQPELLDWEEGASKPKRAGLHGTGVAKVNLLGQQFGGDIDVKLP | |
| FGSGWEVAVSPVFASQQGVGFSGYIEAHQVLGDELTINEEGLARFKASIVGLN | |
| IANKRVYSDLMSLDYNKQQKEVHFAGDAHVPLWDKKKLDGEFDLKLDTAG | |
| KFKSGRTKISSKDTFEVIPKFLSISNPSGEVEVKEDASPEFEVSTDAALYGLPAG | |
| VKASITQAKVMYHDEQLGGEVEQADVEIPISKHTTMTLAMTKAKFTKDAIEA | |
| ENAFMDLDHDNAKVLNETAIVESDFFSGNFDFGKVFELKQLKVHQGLNALKF | |
| AGGKFSYEKDSNNGLQLLRARILGLEAYYNRKDREGGITGEWHKGIDFPAFS | |
| LDFPVAAGVAGVGVDITGGFEFGASIGAKLKNEEQHNTETQMLFSVNGAASA | |
| SAKAKARIEAGAFVGVPYLAKVQGGVFGEIRGGVEGKVETEGRLKYTRGKG | |
| GDSFGQFAIDSDYPMEASFSLTGSLEAEVGASIKAKVLTFEKEIASVSIGDWTL | |
| GEYTLNGKIKKDPNGKGYIVERSKGEFTEGKPKPAEVTKEALPVDKWVNQLE | |
| KDHTHIEYTEDEAKRKLPEVARKTVYSHLSPMQKEDVSERYTHLLKLISQQN | |
| KFTEIYRETKADRDKLPTSGTYIWTSKVWNEAVQRKSFWIFEMSKSKAKVAD | |
| KLDKYHKTHSILERRLLLAKLEEIASDYRKNRSKNAENKEAAGEFLESIEKER | |
| LILM | |
| SEQ ID NO: 3 (chaperone 605 protein) is: | |
| ATGATGTCAGATATGGACGCCTCATACTTGCTTGATAGCGAAGAGCAGAA | |
| GCAAAGAAACAAAGCTCGTCGTCAAAAAAGAAATCGAACGGAAGAAGCG | |
| CCTTCTCGTTTTCGAGAACGCAGCCCAATTCGGCAGCCAAAGCATAACCT | |
| ATCTCGGCAAAACTCAGATATGAGTATGCAAGAAGAAGATATTGAGACAA | |
| TTGTGCTGGATGATTTAGATTACAGTGAGTCGATTTCCGAAGATGAGTATG | |
| ACAACTTTATTCCTATATTAGACCAGTACTCGACTGATTTGACGTCGACTG | |
| AGAGAGTTGAACAAGCAGTTGAAGTCATAGGTAGCAGCTATTTTGATGTG | |
| TTGATGGATGAAGAGCAAGTTTATGATGTCGCACAACGGTACTATCAAGT | |
| GGTTGGCGCGAGGCTTAGCCACATTTACAAACTGCTTCTGCCAGAGAGCA | |
| AGGATGTTGACTTTGCGTTCAAAGCCATCAATCAAGTGGCTCAAAAAGAC | |
| ATGCCTGGAGTTAACTTAATTCAAAACTTTGCTTATCAATACAACCCGTAT | |
| TTAATAGGGACTTCATTCTCAATTAACCCAGCATGGAATATTGCCATACCT | |
| GCTGGTGGCAGCGAAGAAAGGGTACGTGAGACTGCTGGTTCTGATTCGAT | |
| AAATGATACAAAAATTAAGTTTTTTGCAGCCAAAGATTTTAAGTATAAAA | |
| ATGCATCCGGTGCTGATGTAACGATTAGTAATATCGTTGGTGATAAAGTTG | |
| AGGCTCGATTATCTCGTCTAGCCCCCAGAGGCGAGTCTGCTGAAGTCAAT | |
| GCCGATCAAAGCGATTTGATGAAGAATAAGCTTGTTTACAATAACAACGG | |
| GAATAATGCCAATGAAAAAGGCTGGATCCGTGGCCACCTTCTCAACGACA | |
| ATTTAGGCGGCTCAGCATTGAAATTTAATTTGTATCCTATTACAGGATCTG | |
| CGAATAAAGAGCATCACGCTCGAGTTGAATCTCATGTAAAAAACCTCGTT | |
| GAAGCCGGTTATGTTGTTGAATATAAAGTAGAAGTAGTACCAACAGTCCC | |
| CGCACCTCATCAAACAGAAACCGCCCCTGGGTACAAGAGCGGAGCTGCGC | |
| CTAAGGCTAATTTAGTATGCGAGGTAAAAGTCTTAAGTGATATCAGTACG | |
| GATGCTGTGAGTAGTTACCACCCTGAAGGGTCGTTTTCAGTGACCATCACG | |
| TCTGAATACAAAACCAAACATGCCTCTACAGGAGATGTGTCGACTTTAAA | |
| GGCATTTACAAACAAAGCGGTTAAAAGCAAAGACAACACGGTTGACAGC | |
| AAGGCATATATGCGACCTTGGACAACGAGAAAAGGGCTTACTAAGCCGCT | |
| GGGTCTTGCACATAAAGATGATAAACACATTCGAGATAAAGACTATAAAG | |
| ATTGGACAGATAAGCAAAAAAATAGCAAGTTTTGGACATGGGACCAGAC | |
| ACGAATTGATGCGCTTAAAGCGGCACTAAAAGGCTAA | |
| SEQ ID NO: 4 (chaperone 605 protein) is: | |
| MMSDMDASYLLDSEEQKQRNKARRQKRNRTEEAPSRFRERSPIRQPKHNLSR | |
| QNSDMSMQEEDIETIVLDDLDYSESISEDEYDNFIPILDQYSTDLTSTERVEQA | |
| VEVIGSSYFDVLMDEEQVYDVAQRYYQVVGARLSHIYKLLLPESKDVDFAFK | |
| AINQVAQKDMPGVNLIQNFAYQYNPYLIGTSFSINPAWNIAIPAGGSEERVRE | |
| TAGSDSINDTKIKFFAAKDFKYKNASGADVTISNIVGDKVEARLSRLAPRGES | |
| AEVNADQSDLMKNKLVYNNNGNNANEKGWIRGHLLNDNLGGSALKFNLYP | |
| ITGSANKEHHARVESHVKNLVEAGYVVEYKVEVVPTVPAPHQTETAPGYKS | |
| GAAPKANLVCEVKVLSDISTDAVSSYHPEGSFSVTITSEYKTKHASTGDVSTL | |
| KAFTNKAVKSKDNTVDSKAYMRPWTTRKGLTKPLGLAHKDDKHIRDKDYK | |
| DWTDKQKNSKFWTWDQTRIDALKAALKG | |
| SEQ ID NO: 5 (CIS or MAC operon) is: | |
| 1 | TCACGCCTTT TCGATGCTGT AAAGTGCATC AGCAACCTGT TTCAGATCAA CCTCACCTAC |
| 61 | AAAATCATTC GATAAAGTTA AAATAGCATT CAAATTATTT TTGAGTTTTT CATGACACAT |
| 121 | TGGTTCAGGT TTAAGCGCTG TTTGCAGCAA ATGCAAAGAC ATATCGGCTA TCAATAATCT |
| 181 | TTGCTTCTCA GACCAATCAT CATCTCCTTC AACGGCAGTG TGATGGACGA TTGCTTGCTC |
| 241 | AACTTCATCG TGGATCCCCC AACTCAAGGT TAAGATCTCC TGTCTTTTTA CTTTATCCAT |
| 301 | AGCTCCCTCC TATTTCTTAA AAGGTTGCTA CATACTCAGC CAAATTTCAA TTGACAATTC |
| 361 | GTCATATACC AGTTCAATTT TTACTATTTA TTTAAAGGCT CTTAACACTC GCATTCCCAC |
| 421 | TCTACGACTT TGGATTTTTT GGAAAGTAGT TATCCGGTTG CTTAGGACAC AATTCAAATT |
| 481 | CTCGGGTATT TTTGTAAGGC AGCTTCATAA ACCCTGCTAC ACCATAGTCT TGAATACTCA |
| 541 | CTGTGTATTG AATAATCTCC TTTAAAAGCG CTGCAAAGGC GACCTCTTGC TCAGCATCAA |
| 601 | GCAATCGATA ATAGTAGTGA ATTTTCAATC GGTCACTCAG TGACTCAAAA ATAATACAGC |
| 661 | CTGTGTGGTG AATTTTATTT AGCTCAAATA CACACTGGAT AATTTGCTTA GGCAATTGCA |
| 721 | GTTGCTCATC AGGGTCTTTA CCATCTTCAA AATAAGAATG AGGTCGCGTC ACCTCTGACG |
| 781 | CAGCCACATG GCTCACTGTA TACTGCAACT CAGGAAGCTG CTCAAAGATA TAAGCACCTG |
| 841 | TTTCGTCTCG TTCTAATTGA ATTGTTTGCC TCGCATCAAA TAAACAACAC TTAAAAAGAC |
| 901 | CTTTTTGCTC AATCCCCTGC GCCAGCATAA CCGTGGCGTT GACTGCATCT GTAAACGCAC |
| 961 | TTTGTAAACT ATCATCATGC AGCATCAAAC TGGACTCAAC AAATAAAGAC GCTTGCTGCC |
| 1021 | AATGAAAGCT CAGTCCCCCT ACAACCGGAT ATTTAGCTAA TCGACTAATA GAAGCTAAAA |
| 1081 | ATGCTTTTTC AGTGTCCCCC GTATCAAATA GGAACTCTTT ATAATACCTA TCTAGCTGTG |
| 1141 | CATCATTTAC ATCCAGTAAT TGTTGATTAT GATCGGCACG ACGTAAGAAC TGTTTTCTTG |
| 1201 | AACTATAAAC GACAGTATCT GGCAAATCAG TTTGTGAAGA AGTCCAAAAT GGTATATCCG |
| 1261 | CTTTGAGTTG AGGTGAGTCA ATATCAGGCA AGTATACTTT GGCCATCGCA GGCATATTAC |
| 1321 | GCATAAAATA ATCACCTGCT GCATCGCGGA CCACTTTATC TTTACTCAAC ATACCATCAA |
| 1381 | TAACTCTTGA AAACTCCACG GGCAATCCCA AGCTGGTTGC TGGAATTACT TGCGCACCAA |
| 1441 | AGCGACATGA TTGTGCGCTT GCCAGCGCAT AGATAGTCGA AGCAACGCCT TGCTCATCAA |
| 1501 | AGCGCGGAGA AGACCGTGAA CCCGACATTT GTTCATCACC AATAAAATAC ACATCCCCCA |
| 1561 | TTCGAGCATT CGTACTGGCG ATATCAGAGG ACATTAAATC CATGATATTA CTGGCGACAG |
| 1621 | GCTCACCATG TACGTCTATT TGAGCATAAA CAGAGCTACC CCAATCAACC AAAGAGAACG |
| 1681 | CATCAGATTG CTGATCCCAA ACAATATTTG AAGGTTTTAT GTCCCCATGA ACCACAGGCT |
| 1741 | GTAAGGACAT ACCATTTTTT CGTTCTCTCA AGTCTAACAA AACATTGCGT AACTTCAGGG |
| 1801 | CTAAATGAAC CAAGACATGT GGCTTTAGCC GCCCTTGTTT TAACGAAATT TGCTCAAGGT |
| 1861 | CTTCACCTTG GGCTCGAGCC ATCATTAATA TTCCCTGTTT TTTCACACGT TCAAATGCAA |
| 1921 | AAAACTCTGG CACCATAGGA TTATTAATTT GAGATAACAT ATAAGCTTCA TCTTCAAGCC |
| 1981 | GATCTCTTAC GCTCTGCGCC AGCGTGATAC GAGAAAATTT GAATACCCAC TGCGCGCCAC |
| 2041 | TGTCTTCAAC ACCTGCAAAT ACAAACCCAA AGGCACCAGA GCCTATAAGT TCAACATCTT |
| 2101 | GATACCCAAG CAACGACAAT TGCTTTTTAC AAATGTTTAG CCATTGACGG TGTTTTTTTG |
| 2161 | CGTCATGGTG AGATAGCAAA TATATCGATT GCTGTTCATT AATGTAGAAA TTGTGAATTG |
| 2221 | ACTGTTTGCT CACGGCGCGA CTTAATAAAA CTAATTTGAT GAATATATTG AAAAAAAGGC |
| 2281 | CGACAAATGT CGACCTTTTT TCATAAATAA TAGCGTTAAA TTAAACTGCT TTACGCTTTT |
| 2341 | TCAATTTTTG CCCATGAGTC ACGAAGACCC ACCGTTTGGT TAAACGTCAA AGCTTCAGAC |
| 2401 | TTATTGTCTC GGCTGTAATA GCCTAGGCGT TCAAACTGAA AACCTTGTTC AGCATTCGCT |
| 2461 | TTGGCAAGTG AGGGCTCAAG TTTAGCATTT GCAATCACAA CCAATGAATC TGGGTTTAGC |
| 2521 | GTAGTTGCAA AATCTTCTGC GGCAGCTGGG TTTGGTACAT TGAATAGACG GTCATACTGA |
| 2581 | CGTACTTCCG CTTCGATGCA ATGTGACGCT GAAACCCAAT GAATTACGCC TTTAACTTTA |
| 2641 | CGGCCATCTG CTGGGTTTTT ACCAAGCGTG TCGGCATCAT ATGTACAATA AATAGTTGTA |
| 2701 | ATATTGCCTG CATCATCTTC TTCAATGCGC TCCGCTTTGA TCACATAAGC ATTACGTAAA |
| 2761 | CGCACTTCTT TACCCAACAC TAAACGCTTA AACTTTTTGT TCGCTTCAAC GCGGAAGTCT |
| 2821 | TCACGTTCAA TGAAAATTTC ACGAGTAAAT GGCAATTCAC GCTCACCCAT TTCTAATGTT |
| 2881 | GGGTGATTTG GAGCAGATAA CATCTCCACT TGGTCTGCAT CATAATTTTC GATAACAATT |
| 2941 | TTAACTGGAT CTAAAACAGC CATCGCACGT GGTGCATTTT CATTTAGGTC ATCACGGATA |
| 3001 | CATGCTTCAA GCATCCCCAT TTCAACCATG TTATCTTGCT TTGTAATACC AATACGCTTA |
| 3061 | CAGAATTCAC GAATAGATGC CGGTGTGTAA CCACGACGAC GTAGGCCCGC AATGGTAGGC |
| 3121 | ATACGCGGAT CATCCCAGCC TTCTACCTGA CCGTTCACGA CTAAGTCATT AAGCTTACGC |
| 3181 | TTGGACATCA CGGTATATTC TAGGTTTAAA CGAGAAAACT CAATCTGCTG CGGTTGACAC |
| 3241 | TCAATGCTGA TGTTCTCAAG TACCCAATCG TATAAACGAC GGTTGTCTTG GAATTCAAGC |
| 3301 | GTACACAATG AATGCGTGAT CCCTTCTAGC GCATCTGAAA TACAGTGCGT GAAGTCATAC |
| 3361 | ATTGGATAAA TGCACCACTT GTCACCAGTC TGATGGTGAT GAACAAAGCG TATACGGTAA |
| 3421 | ATAATCGGAT CACGAAGTAC CATAAACGAG CTTGCCATGT CGATTTTTGC TCGCAACACA |
| 3481 | CACTCGCCTT CTTTAAACTC GCCGTTTTTC ATTTTTTCGA AAAGTGCTAA GTTCTCTTCA |
| 3541 | GGTGAGGTAT CTCTGTGTGG GCTATTTTTA CCAGGCTCAG TCAGTGTACC ACGATATTCA |
| 3601 | CGTGCTTGTT CGGGAGACAA GAAGCAGACA TATGCGAGGC CCTTTTCGAT CAGCTCTACA |
| 3661 | GCATAGCTGT ACAATTTGTC GAAATAGTTT GATGAATAGC AAATTTCACC ATCCCATTCA |
| 3721 | AAGCCTAGCC ATTTAACATC TTCTTGAATT GAGTTTACGT AATCGATGTC TTCTTTTTCT |
| 3781 | GGGTTAGTAT CGTCGAAACG TAAATTACAA AGACCTTTAT AGTCCTGAGC AATGCCAAAA |
| 3841 | TTTAGGCAGA TAGACTTTGC ATGGCCAATG TGTAAAAAAC CATTCGGCTC TGGCGGGAAA |
| 3901 | CGCGTATGTG TCGATGCGTG TTTGCCACTT GCCAGATCTC CGTCAATGAT ATTTCTGATG |
| 3961 | AAGTTTGTTG GGCGATTCTC TGTATCCGCC ATAGGAGGTC TTTCCTCTGA ATTTGTAGCT |
| 4021 | TGTTAACTGA CGCATTATTA CAAATTAATC AGGTAACTTA CAGCGCTTAT CGACCCTTAA |
| 4081 | AGTCGATTTA TTTAATGTTT TTTGTATGCT GCACAATTTA CATACAACTT CATTAGTTGT |
| 4141 | TCCTTCCAAG CGGTTTTTCA AGTGGGCATC ATGCGATAAC AAATATCAAA ATAATTTTAA |
| 4201 | AAGAATGACA CCGCGCTAAC TCACTTTGCA ACTCACGCCA TTAACGTCTT CAAAAACCAA |
| 4261 | AATGATAACT CCCAATTTAC ACATACCACA GTGTATCAAG CTTATTCACT AATGAGGTTC |
| 4321 | ACCCATCACA GACGGTTTGT TTCATTTTTT CCTCATTGGC ACAAGCAAAC ACCTCTAGAC |
| 4381 | AACAAGCTCC GCTTATTAAA ACTAGAAAAA GCGCAAAATC GCCTTATGCC ACAATTAAAA |
| 4441 | ATAAAGCATA CAATTTTTTG TTCACTTTTC ATTCATAATG TGTTTTATTT ATAGGGTCTA |
| 4501 | ATCCAACTTA AATGGAAATA GGTTGGGCAG TTAAGTCAAT TAGAGGTAAT CAAGGAGATA |
| 4561 | TGATGATGAA AAGTATGTTG GCACTCTCAC TAGGTTTGAG TGTGTCTTTT AACGCCATTG |
| 4621 | CGCACAGCTC AGAGAAAAAA GTTCAGAACG CTAGCTATAA GGTTTGCCAT TACAAACTCG |
| 4681 | CGGCGGCTAA GACTAAAGTA TCTTCATACC CGTTATGGGA GAGAAAAAAA GGCACACGTG |
| 4741 | GCAGAGTTGT CCACTCAGTT GGACGCTTTC TGAACCCGAG AATAAAGCAC CAATTGGCGT |
| 4801 | GTACATTTAT CTTTATACGA CATACCACTA AACTTATTTT ATTGCGAAAT CCGCCACACC |
| 4861 | CTTGATGCGT TAAATGAAAT AATACTGCCA TAACATCATT TTATAGAGCT CAATAACGAT |
| 4921 | GAGCTCCCCG ATAAAAATCC ATACAAATTC ATTAACTTCT AAATGTATAG AAAATAAAAT |
| 4981 | AAGATTCAAA AGTATTACTT TTTGCTATTT TAGTACATCT TATTTTTCTT GAATATGCTT |
| 5041 | ACACTTTTTT GGACATATAC AGCATGTAAA GGAAAACTAA ATGCAAAATA CAACGAAAAT |
| 5101 | ATTGAGTATT TTAGGATTAA CGGCCACCAT AAGCTCTTCA TTCGTCTATG CTCAAGGTCA |
| 5161 | TGAAAATTCG GCCATTCGAG TTATGATAGA CCAACAAGGG ACCCCCTATC TTCACAAAGA |
| 5221 | CGATAATGTT GGAACATGGC AAGCAATTCC ACTGCCCTAT TATCAAAAGG CTGTAGCCGC |
| 5281 | TTCCGGTGGT TATTTTCACT ATCGCCATGA AAGTGGCAAC AGTCCAGGTT ATGCCTTCGA |
| 5341 | TGAAGAGGCC ACATTCGCAG TAGGCGAATA TGGCATGATA TTTAGATATC AAAACGATGC |
| 5401 | ATGGCAACAC ATTTTTGGAT GTTGGGATGC GAGGGATGTA TCATCTCACA GCCCTAAATA |
| 5461 | CGCTTATTGT GTTAATGCCA GTGGTGATTT GAAACGCTTC AATCTTAACA ATGGTAATTT |
| 5521 | TGGTGGGAGT TATGGCATTG ACGGCAAAAA AATCACCAAA GTGGATGTCA ATCGACAAGG |
| 5581 | AGATGTTTGG GCACTCACAC AAGACAATGG AGTTTATGTG CGCAGAGGCG ATCACTGGTC |
| 5641 | ACAAGTAGAG GTTGTCTGCC CTAGAGCGTG TTCCTTTAAA GATATCGCCG TCGGCGCTGG |
| 5701 | CCAAATATAC CTCACGGCTC ATATCGTTAA AAATACCTTA GGCGAGCAAC AAGTCTACCA |
| 5761 | ATTAAATGGC TCGAAATTAG AAAAATTCGG AGATTTTTAT AATATCGAGG TTGATAGAGA |
| 5821 | TAACACCATT TGGGCGATTT CAAATGAATC TAGGACACTG CACTACAAAC GCCCCGGCAT |
| 5881 | GATTAACTTT ATTGAAGATC ACCGTATAAA TAGTGTGTCA GCCAACGACA TAGGTGGTTA |
| 5941 | GTTTTACTGC GAACTTACAT AGCTTGAGCG CTTAACGCTA AAAAGTGCCA AGCTATTTGT |
| 6001 | GTGTAAAACC GAGCGCATTG AACTTATTTG CCACTCGGTC TCCACCAAGC CATTAGAAAA |
| 6061 | AAGATGACTA AGCCAAAAGG TATTAAAGCC GCAATACTAC TAACATACCC TTGTACATGC |
| 6121 | ACTAGCTTTT CAAATGGTGC AATGACGCGT CTATTTTGAG TGATATTTAC TATTTTATTA |
| 6181 | TTATTTGAAG CGTCATACCA GACTCGTACT CTGCCATTGG GTAACCCTTG CTTAACCTGT |
| 6241 | CTATAAAATG GAAAATATTC ATTGACAGTA AAAGGGACAT TCTTAATCGT AAATTGAATT |
| 6301 | CTTTCAGAGC AATTTCGTCG CTTGTTGCAC GGTATTTCTT ATACAATGAC CGGCGCAGCT |
| 6361 | TGAGACACAA TTGAGAAATC CGCCAGAATC GGTCGATTAA GCTCTGTTAA ATATTGTACT |
| 6421 | AAAAAAGTAG CTATTAACCC TGCTACAAAG ATAACAATAG AAATGGTCAC TTTTTGCATA |
| 6481 | CTTTCACTCG CTTTAAAGCG CGACATTCAA AATCGATCCT AATTGTAAAA AGCTGCCGAA |
| 6541 | GCAGCTTTTT AAGTTGTAGT CAAATAGGCC AAACGCTGCT TAAATCGTTC ACTTGGCACT |
| 6601 | GTATTCCACA GCTCTCTATC AGACATCTCT TTACTAAACG CCTCAAGGTC ATTTTGTACT |
| 6661 | ACATAGCGCT GAATTCGTCT GCGCGTTGCT GGGGTGTAAT CCCCTCGGTA GCGTAAATCA |
| 6721 | ATCAAAATTT CCTTGATCTT GGGGTGAAGG TTTTCCCAGT CCACGGGCCC ATAGACTTCA |
| 6781 | ACACAATCAG CTTTATCACA TATTCTCTTG ACGTCTTTTG ACATGACTTC GTAGGTTTGC |
| 6841 | TCGAATAAAA TCACTTGCTC CTTTGGGCTC ACTTCAAAGC CTTCAAGACC ATGAGTCTTT |
| 6901 | ATAAACTGCT GTGCCTGTGA CCCATATAAG CCTGAAGCTT TTGCCAAAAC TTCTGCATCC |
| 6961 | TCAAAGGCAA CGCCAGCTTC GCTGAGCGAT TTGAGTATTT GTTTTGAGCT GCGATGTTTG |
| 7021 | AGGTCAAATC CTCGGCCAAT TGTCAGGCCA GAATACTTTG AAGGCACATG TAACACGCGA |
| 7081 | CTATGGTAAC GCCCACCTTC TTGCCCTTCT TGTTCAAAGG TAAATTGGCC AACTTTCGGC |
| 7141 | TGTTGTATTG TCATAGTGCT TTCCTTAATT GTTCTAGTTC ATTGCTTAAA CTAGTAGTAT |
| 7201 | AAATGACTAC CTATATTGGG TAATAAACCC ATAATTCAAG TTTTAGCAGC AAGAATCATG |
| 7261 | TTTTCTAATC GCCTATTTTC AATGAGCGAG AAATAATCAA CTCCTGCGAT GAGCCACTCG |
| 7321 | ATGAGTTTGT TTTCAGGTAA TGTGTTTATC TGATTTAAAG TACACTCACA CATTTGGCTA |
| 7381 | CTTAAATTAA TATTAAAGTA GTTTTGCATT TCACTGAGCA TAACTGAGAG GTAGCCTTGA |
| 7441 | GCATTAATTA ATAACAGTAA CCCTGCGGCT TGTTGATTCT GGATTTCCTC ACCTTTGAGC |
| 7501 | TCTGACCAAA GTTCAAATGC ATAAATATGT TCAAATATTC GTTCAACCTC CTTTTCACTA |
| 7561 | AACCCATGGT TATACAGACT TTGCCAGTAA ATCAATGGGT CTGAATTACA ATATAAACTT |
| 7621 | TGACGCGTTT CGCTCGTGTT AGAAAACGCA TATTGAAATG CCTCTTGATA ATTTGCCACT |
| 7681 | GTGACTCTCC TTAGCATATT TAAACACCGA CGCAAACAGA TGAGTTTTGT GACAAAAAAC |
| 7741 | CAAGACGTGT ATTTGACCTT TTATTAGCTT AACTTCCCTC TGATCTGTAA AAAAACCAGG |
| 7801 | CTTAGAAAAT CCGATGCCAT GGACTTAAAT TCACTGTGGT CCTCAAAATT GGGCGGAAAG |
| 7861 | GGTAGATGTG GAAACTGCAT ATCCGCAGTC GGTATATTTT GATAGTAATA ATCATCAGCA |
| 7921 | AAAAATAGAG GCTGAGTCTG GCTGGTAACA AGATCTGTAA TATATTTTGC ATATATAAAC |
| 7981 | GCATGGTTCG GCCTTGTAAA AATCAACAAT CTATTTTGCG TTAAACCAAT ATCTTGATCG |
| 8041 | TCTTGATTAC CTGAGAATAA TAAAGATACT TGCGTGATCA CTTCAATCGC TGAACTTTCT |
| 8101 | TCATATAACG TCTGTTTTTC TATCTCGATA TCTGCGGTAT ACCCTTGTTC AATGAGTAAC |
| 8161 | GATTTTACTA ACTCTAGATG TGGTAATACC CTGCTTGTAA ATAAGTTATT CAAATGTTGG |
| 8221 | AGCAAATACC GACGATTATA CTCCAAATAA GCTTGTACTT TATTTTCCGC AACTTGATAC |
| 8281 | TGCTGTTGGC GCACCTGATG TTCCTCGATT AAGGCGCGTA GGTTTGAATG ATTATTTACA |
| 8341 | GGCATACACT TCTCCATATT GAATTGGGAC TGGCTCAGAG TTTGCGAGTC CATTTGATAA |
| 8401 | TTGGTTTTGG GCGTTGAAGC GCACCACGTC AAAAGATGCG CTTTCATTTT AAGTTTTATT |
| 8461 | GTGAATTTAA GGTCATGGCT TGTGAACGCT ATGCTAAAAG CCGTAATCCC ACTCGATGTT |
| 8521 | AATAAGATCG GTGTCTAACC AAGGTAATTG CACTATCCCT AGGCCCCAAG GTAGTTTCAT |
| 8581 | AAGTAGTACA TCTTGAGGTT TGTTCTCGAC AACAATACTC ACACCATTGG CAGTGAGCTT |
| 8641 | TACTTCTCCC CTCCTCTGCA AGAACATGTG TCTAAATGCA TCGATGGGCA TATCTTTGAG |
| 8701 | CGCTTCCCAG CGGTTTATCG CAGCTTTAAT GAGGGTGTGT AATTCATCTT GTGCTTCGCT |
| 8761 | ATCTATAACA AGCGTCTCTT CATCAAACTC ATGGTCAAGT TCTAGGCCTA GCAAGGCATT |
| 8821 | AATCACATAC GTTTCTGCGC TATTTGCCTC AATCCCAGCC AGTTGACACA ATAAAGCATG |
| 8881 | CGCTTTTTGT TGAGCAGCTA AATCCGCAAA CGTGATCTGA CCGCTTTCAG CTGTAATAAG |
| 8941 | TAACTCTAAC TTAGTGAATA AAATTTCTAA AAATGGCCAC AGCAGCACCA CACCGGCGTC |
| 9001 | GTCACTAATA AGACTCTCTG TTGATTGCTG ATATTGATGT CGTAGCTCAT TTAACATGTG |
| 9061 | TTCACTGTGC GCTTTTAAAG GCTCAATACC AGAATGTTTC AGGTTATTTT GATACCTACG |
| 9121 | TACCTGTTTT GACAGGCATG CAGAAATACG TTCGACCCGA CGCTGTTCTT GTAGCCGCTG |
| 9181 | CGACATCTCT TTTACGACTG AGTGTGATAT TCCCTTTTCA ACCACTTTAA GGTCTGGTTT |
| 9241 | ATTGCTCATA TCTGTGCGTA TTGCAGACAC CGAACTATTG GTGCTTTGAC CCAGCTCTGC |
| 9301 | GCAGTTCAAT GCAACCACAG CATCATCATC CGAAGTCACC TCCCCAAGCA AGGCGCCCTT |
| 9361 | TTGTTTTTAG TTGCTTGAAG CCGTTCCTCA TAATGAGTTA ATAATCGAGT CACATCGGTA |
| 9421 | AAAGTTCGAG CTTCAACATA TTCACTGATG TCATGTATAG TAATAAAGTT TTGTTCAGAA |
| 9481 | CTTGTTTGGT GAACTTGCTG ATGACTTCCT GCGTTCAAAT ACACATCTGA CAGGCCATCA |
| 9541 | TGGTGTACAT TTACTAAACT TCCTGAGTCA TCTTTTGCAA AGTGACTATC AGGTACCAGT |
| 9601 | GATACATCTA ACAACTCTAG CCACCACATT TGCCCTTTCT GTGCTTGCGT TTTCAAATAA |
| 9661 | GGGTTTAGCT GATAGCTCTC CTCCCGCACA GTATTCAGTT TGACAATTGC ACTATTCAAA |
| 9721 | ATCTCATTTA ATGACGGTGT ACTTAGTGAT AGACAATGGA TCGAACACAC TTTTTGCCAA |
| 9781 | CTGCGCAGGT ATATCACCAT ATACACAAGC AGTTCATGAT TATCGTGTAG CTGGATACTC |
| 9841 | GCTTGATTGA CTTTGATAGT GTGCTTAAGT GCATCACAGT ATCGTAATAA TTGAGAGGCG |
| 9901 | ATGCTCAACA TTCTGGCCAA TTCAAGACTT GAAGATTTAG ACAGCGACTG ACGAGCTACT |
| 9961 | TCTGGTACCG TTCCACTGTT ACTTGATTTA TTTTTACTAC TTAAACTGTG CTGCGACTCA |
| 10021 | CTCAGTGCTT GCTGCTGCTC TTGAGCTTGT GACTGCATTT TATCAGCTAA CTCACTTTGT |
| 10081 | AATTTTTGAA CGTCACGGAT CTGATAAATC GCCTGTTCAA GGTCTTGACT AAGCTGTTCA |
| 10141 | CAAACAGAGA GCAAACTTGC AATTGAAGAC AAGCTTTTAG CAGTGTGTGT ATAAACACCG |
| 10201 | TCGTAACACT GATTGATATT TTGCGTTGCC AAAACCGTCG TTGAACTCAA ATCTGAAGCA |
| 10261 | TATTTTCTGA GCAGCGACTT TAACTTTTCT AATCTTGCAC CTTTCAATTC GGTGTTCAAT |
| 10321 | ATCAATCTTT TACTGTCATT TAATCGTGGA GCTACCGCCA ACAACGTTAA TCTGAGCTGT |
| 10381 | GCAGCCTCTA CCTGCTCCAA ATATTTTTCA GATGTGCTTC CACACACAAT TCGCAGTGAA |
| 10441 | GAGTCAAATT CACTTACGCT AGTGAGAGAC AACGGTGTTG TAACTTCAGA CCCGTCTTCA |
| 10501 | AGCTTATGAG TGTTTTTTTC ACGCCGCTCT ATTAAGTTTG AAGTATCGTC TTTTAGGAGC |
| 10561 | AAATGATTAG CCACTGAACC TTCGCTGATA CGGATGGCAC TGTGCTTACC CAGTATGTAT |
| 10621 | TCATTATCTA ACCAGCGTTC AACCTGTTGA AGCAGTTTTT CATAGCTCGG TAACGCCACA |
| 10681 | GCAAATGTAA AGTCACTGAT TGCATGAATT GAAGGTCCGA ACATACTCTT AAATTGGTCG |
| 10741 | ATATATTGCC AAGGTATATT TTTGATTTGG CACAACACCG CATATAAACG ACGAGCATAT |
| 10801 | ACCGGCCTAG ATTCAATTAC TTTTTACGTT TTATCCATCT GTATTCACGA GCGATCAATG |
| 10861 | CCAGTGCATA ATTAACAGCT CGGCGCTTAG ACAACAAGAC TTGCTCCGTA CCGAGCTGTG |
| 10921 | ATTTCAACAG GTGTTTCAAA GGGGTCAGCA CAGATGCTGG TAAGCGCTGC TCAATATCGT |
| 10981 | GCTGAGCAAC AAACTCACGC CACAGCACTT GACTATAAAA AGGCAATTCA CCGGCAGTAA |
| 11041 | CATGTGTATC AATAAGCTTA AGTAACCCTT CTAAGGCTCG GATAGTTTTT CTGCAACCAC |
| 11101 | TACCAAGCTG CCCCTGCAGC TGAGCAAGCA AATATGAAGC CTCTTTTTGA CGCGTTTGGT |
| 11161 | TAACTGTCGT TTGTTGCTCT CCAACAGGAC TATAATCATC CAACAGTTTA GCTAATCCGC |
| 11221 | TAGGCTGATA AGGGGTTATC AGTGCACGAA CACTTGCCAC AGCCCTTTCC TTAACCTTTG |
| 11281 | CTTTATCAAT CAGCTGCACT AATGCCTGAC AGTCAGAACG GGTCAATTCA GTCGAAGCTT |
| 11341 | TAACCGTGAA CCAAGTTGAT AAATAGCGAA GAGATATATT GTTGTTTTTT ATTCCACTGA |
| 11401 | CCACACACCT TCTTATTAGC TCTGAAAACT CTGGCTGCGC TACAGACATT TGAAAGCTTT |
| 11461 | TTAACACGAG CGATAAACTG TGCTGGCGAG TATTCAAATC TGATGCATGT ATGATCCGAA |
| 11521 | GTGTTTCGAT ATCCTGCCAT AGCTCAATGC ACTCAGGTAG TGACAACTTG GACAAAAATG |
| 11581 | CCTCAGAGCT TCTAGAAATG AATCCATCGC TAATAACAGA AAATGACGCG GCAAGGTAAT |
| 11641 | CAGGATCTTG ATATATCGCC TCGACAAACC GCCTTCGCAT ACTTGGCCAT TCTGCACTGT |
| 11701 | TGAGCACCAT CAAAAATAAC GCTGGCTGGT GCTGTAAACT ATGCCAACAA GCCGCCAATA |
| 11761 | CTCTTTGCCA AATATGCGCA CTCTGCTCGC TTTTTGCACG TTTTAACACA TCGTTCAACA |
| 11821 | TCAGCGGCGC CAAAAGGTAA TTCTGTTTTT GAGCGCCACT CAAGTACTTA ATTAATTGCT |
| 11881 | GCTCAACCGC AGTATAAAAG GCAGGACTAA ACACCCTATT AAAATAATTA CTTGGGTCAC |
| 11941 | GTGCTAACGC ATTTAGGTCA ATATCTGGTA GCTCTATTTC AATATCTTCC AAAGTCACAG |
| 12001 | GCGCGATACC TGACTCCGCG ATTGGCCAGT CTAATTCAAG TTTATTTATT TCCTTATACA |
| 12061 | ACCAGTTTTT AAACAGTGAC TCTAGGTCAG CAATACTGTG CTGACCTAAA CTCACTAAGT |
| 12121 | GTGCAGGGGC TTCTAATTCA GCCCCTATTT GACCTATTGC TAGCTTCATG GTTAAACGTG |
| 12181 | GTCGTTACGC CAAAAGAAGC GAGAGTGATT CATAGGCTTG TCTAAATACA GCGCATGCCA |
| 12241 | CAAGAAATCA CCATGGTGTT CTTCTTGCTC TTCGAGGTGA TAGCAGCCCA TGCTAAAGAT |
| 12301 | ATGGTCTCCG TCACCTTGCT GTGCCGTATT ACGTCTCCAG AACCCTTTAC AGATCAAGTT |
| 12361 | TTGACATTTA TAAGCGCCGC CACCATGTAG CTGTGCAGTA TTATTGATTG CAACTACTTC |
| 12421 | ACTGTCATCA CAATGTGCAA CAGCACCTCC GCGTTCGGCT TGGCAATTGT GTGCTTGAAT |
| 12481 | ATTGGCATAT CTCAAGCCAT AAGCCGCCCC ACCTTGACGG CCTGCTCGAC AATTGCGGAT |
| 12541 | ATTGTTCGCG GTGATGTTAG TCACATCTTC GCCATAAATC GCTCCACCAT CACCACTGAC |
| 12601 | TGCATGGTTC TCAATCACAC AATTAAGTTG TATTTGATCG GCGTAGCGTA AGTTAAATGC |
| 12661 | ACCGCCGTTT CCTTCATAAG CACTGAGCTC TCTGGACACT TCGTGGATTG CACCATCAAA |
| 12721 | TGTGAAGCCG TGCATTTCAA CATGTTTAAC CATCGCGTCT CTGTCACCAC GGATCAAGAA |
| 12781 | GCGACATCCT GCATGTGCTT TGACAATGCG TGTTTCACGC TCATTAAAAC CTACAATGCT |
| 12841 | CAAGTTTGAA CCGACTTCAA CGGCATTTTT CAGTTTATAG GCTCTAGAAA CCTGGCGATG |
| 12901 | ACCATTGTGT GGGAATAATA AGATCGTCAT ATTATTCAAG CAATCTCTCG CAAATATCTC |
| 12961 | GTCAAACTCT TCTTGATTAT GGATCGCAAC AACCTTCTTA CCGCGTCCAA AGCGCCATGC |
| 13021 | ATGTTCATCC ACATAGGCTT TAACAGCAGC TTGAGTGGCA AGTACATGAT CTGACGCCAT |
| 13081 | CTCACCGCCC AGTTCACAGT CTGACGATAT TGCGTCAATC TGCGGCCCTT GTGGTAATTG |
| 13141 | AAGTTGCTGT GGTGCAATCT GGCCTTGAAC AGCACAATCA CCCACAATTT CAGCCCCCTG |
| 13201 | TTGTAGGTTA AGCTTACTCT CGAGGACCAA CTCACCAGTC ACAGTCAACG AGTCCGATAC |
| 13261 | GCGTGCATCG CCACGGATAT CTAACTTGTG CTCAGGTGCT TTACAGCCAA TACCAACATT |
| 13321 | CGCATTGCCA TCAACAATCA GCTCTGAATT ACCGCGCACA GCCACATCTA GCGCAGCTTC |
| 13381 | CCCTTCACTG GTATTAACTG ACACCATCGC GCTGTCATTA TGTGAAATGC CCACTTTAAC |
| 13441 | ACTGTGTTCT ACCGCTAAGC ATGCAGCATT TACGGCACCA CTGATATACG AATCACCTTT |
| 13501 | AACATGCGTG TCGTTACCAA AGTTTGTTTC ACCTTGCACG GCTAAATGAC TATGAATATC |
| 13561 | TACATGCGGT TTAAACACGA CCTGATTTGC CGCATCTAAG CCTTCAACGA CTAAGCTGCC |
| 13621 | ACGTACCGTT GTATCTTTAT CAAAGTGAAC CGCTTCTTTA AAGAATGCAT TACCCCTTAC |
| 13681 | TGTCAGCTCC TCACCTAGGT GCAAGCTTTT AGCAAAGTCG GCGTCAGCCA GAAGATTTAG |
| 13741 | TTTGCGCTCT ATGGTTGTTA GGCCCTCAAT TGTGACAGTA TCTTTAAATA AGGCATCATC |
| 13801 | GTATACGGTC AAGTCAGAGC GCAGCTCCAC ATCACCTAAA ATCTTCGCGC CATCTAAACA |
| 13861 | CTCAAGTTGG CCGGTACCAC GCAATACCCC ATTGAGCTCA ATATCACCTT CGACCTTAAT |
| 13921 | ATCACCTAAA ATATCCAGCT CAGCTTCCGG TTGTGCATTG CGGATCCCCA CTTTGCCATC |
| 13981 | TTGAAATACA ATTGGGTTAA CCCCTTTGTG ATAAACGGCT AAGCCATACT CAAAGTGGTT |
| 14041 | TTGCTCGACA CTAAACTGGG CGTTACGGCG TATTGTAGAT GCGTCTTCAT CTTGCGCGTG |
| 14101 | CATAGCCAGG CCAATTCTGG TTTCACCCTG AAATTCTGAA GTGCCATAAA TGTTCACATT |
| 14161 | ACTTCTGAAG CTGGCATACT CATTGACGTG TAGACTATCA TCTATTTCGA CGCGACCATT |
| 14221 | AACAGTCAAA TCCCTTCTAA AACAGGCATC TTCCCCTACA TCTAAAATGT AATCTGGGCG |
| 14281 | CTCTACACCC AATCCTAAAT TGCCATGGCT AAACACCAAC TGTGCCTTAC CACTTGGGTC |
| 14341 | ATCTATACGA AGTGCTTGTT GATCCGTTTC ACAGATATGC ACTCGCGCAG TCGCTTGATA |
| 14401 | TGGTGCTAAG GACAAATTTC CCGAGATAGT CGCATCGGCT CTAAAACTAG CATCATTATT |
| 14461 | TACGGTCAAC GCATCGCGAC CTTCAGTGCC CTCAATTTCG ACACTACCAC CAGCATAATG |
| 14521 | CTCGCCAGAT ACTGTTAAGT TCTCCCCAAC TTGAGCATTG CGTCTCACTG CCAAGTTAGA |
| 14581 | CTTGATTTCG CTATTACCAT CTACCGTGTG ATTAGCATCA AACTGCACGT CACCCGTTGT |
| 14641 | ATGTAGTTCA CCCTCTAGTA TTGATTCTCC AGATACTGTC AGAGCAGCGC GCTGTACATC |
| 14701 | TCCTGAGTTG TTAGTCACAC TCACATGCAG TCCGTTTTTC AACTCTGCAT GACCCATCAA |
| 14761 | CTTAGATTTT CCGTCTACTG TTAACGCAAT CCCATTATCT CTAATAGTGA GTTGGCCCGT |
| 14821 | AATATCAGCC AAACCAGTAA CCGCAAAATT AACGCCACCT TCTGGGATCA TTTGATTAAT |
| 14881 | AGCAAGCTGG CCTGCAGCTG CCAATATTTC CGCATCTTGT GCTTGATTAG TGATTCTAAA |
| 14941 | TACGGGGTTT TCTGATAATT GAGTGAGCTC AAATTGGCTA TTCGCAAAAT TGACTGCGAT |
| 15001 | GTGCTCGTTT TCAACAATGG TAAACTCATC AAATAAATGT GTATTACCAT ACACGTGCAA |
| 15061 | ATCTGCGGAC TTCTGCTCAG TACCCACTTG AGTTTGTATC AAGGTCGCAC TGAAACCACC |
| 15121 | AAATTCACTG TTCTCACTGG CAACGTAAAC GCCTTTAAGG GCCGTGATAT CTTTATCAAA |
| 15181 | CAATGCAGAT TCACCGACAT GTAAGCTCTG CTGCGGGTTA TTTTGGAACA AACCAATTTG |
| 15241 | CTTGCCCGTT GCATTGATGA CTGACGTTGT GTTTAAGCCG TCAAAAAAGC TGACATCCAC |
| 15301 | ACTTTGTGGT TGGCCGTTGT GGGCTAGCAC AGCCAATTTT GCGTTGGTTG TCCAATCACT |
| 15361 | TTGAACCGTG TTAGATGCAA TCACCACATG TTCACTAAAG TCCGCCTGGT AAATAGTCGC |
| 15421 | CTCATGAGAA ACGTCTAGAT CAGGCGTTGT GATATCCCCT TTAACCGTCA AAGATTGAGC |
| 15481 | TTGGCCTGTA CTACCCATCA CTGCTTGGTC ATTATCAATA CGCAGTAATG GATTACCTTC |
| 15541 | TTTATCAGCA ACTAGCAAAG ACGGGTGATT TGTGGCTGTA CTTTTCACCG TCAACGTCGC |
| 15601 | TTCATGGTTG CCTACCTGCT CACCCACAGT GAGATTATGC CTCACTACAC CATCGGTTGC |
| 15661 | TTTTACTAAG GTATCGGTCG AAATCTCCTC ACTCGCATGC AATGATTTAA ACGTAGCATG |
| 15721 | TGCAGTGACA TCGAGGGTGG TATTAATAAG TGCGCCTGCA TCTGTTTGTA CCGGTGTGCT |
| 15781 | AGGCGTTTCG CCGACTTGAA GCTGGCGCGT ACGTGTTTGA CCGCTAACTT GGGCATCTTG |
| 15841 | ACTAACCGAC AGCGCATCAA GCACCGTACT CCCCGCTGTT AAGGTATCTG TAACCTGCAC |
| 15901 | AGTCTCCGCA TTGACTTGTC CAGCAGCAGT CAAATCATCT GAAATGTATG TATGTGCAAA |
| 15961 | CATATTGATC GACTGTTCAC CCGCTAGTGG CATATTCGTG ACTTTCACCA AATTACGACT |
| 16021 | ACCATCACCA ATATTCAGTA AGTCACCGTT GCCAGCCTGA ATATCCACTT TTGCATTCGG |
| 16081 | TGTTTGCGCT CCCACCGACA GCTTGCCGTT GACATACGCA TCACTTTCTA CGTTCAAATC |
| 16141 | GCCCGTAATG TCTGCTGCTT GTTCTATGCT CACAGTCCCA GAAAAGTGCG CGCGCCCGTC |
| 16201 | TTCATTTAGC GAGATGTGAG GGTGGTCTTT ATCTTGTCCA ACTAATACCG AATCCGCAAC |
| 16261 | ATGGAAGCGC GCTTTAGGCA CACTTGTACC CACCCCCACT TTACCTTCTG GGTTGATAAT |
| 16321 | AAAAGGCGTG TTATCTTGGT TTAAATCATC CACTCGGAGT AGATCGCTCG TGTCTGATGA |
| 16381 | ACGTTTGCCA ATATGCAATT TCGCCAAGGT GCTATTTTCA TCTAGGCCAA CACCTAACAT |
| 16441 | CGGCGCTTCT ACATGCTCTG TAAAGATGGC ATTTTGCGCA CTGAGCTCAC CGAGTAACAT |
| 16501 | GGTACGCTCT GCGACCGATA GCATCTGAGT TGATGTATTA CCGCTCACAC TGGCATCATT |
| 16561 | CACAATAGAG ATTGAGTCAG CTTGGATATG ACCTTCAACG GCAACATCTG CGTTCACTTC |
| 16621 | AACATCACCA TAAGCGGTAA TTTTTGGATT ATCATCATCA TCTAAGCCCG CTTCCAATGT |
| 16681 | CTGGATAGTG CCTTTACTGA CAGATAACGC GCCCGCTCTT AAGTGCTGAT TAATATCCGC |
| 16741 | CGTTTGTGAC TTTAAGCCCT CCGTTAACTT TGCGCTGCCA GAGACGCGTA AAACCTCTTC |
| 16801 | TGCGTCAGGT AATACTTTGA CTTTGTCATA CACAGAAACG CTTTGCGCAT CGGCACTGAG |
| 16861 | CACCTGCATT TCGCTATCAC CATCACAAAC AACCAGTTCG TGATCAATTT TGACGGCATC |
| 16921 | AGTATCCACG CGTAAACGCG GTGTCTCACC ATCTGGATTG ACTGTGAGAG GCGATGTAAC |
| 16981 | CGCTATTTCT TGAGTTTCTC CGACTCTCTC TACTGCGATC GGGTCGTCTA TTTGATTAAA |
| 17041 | GCCTGAGCGA ATAAGAGATT CAAAATCATC CCCTGTCGGC GTTTTGAGAT CATCAAACTT |
| 17101 | TTCAATTAAT TCTGCGCGGC TGCGCTTAGT TACTATAGAT GTTTGGTCAC ATTCGACTAA |
| 17161 | CACTTCTTGG TCACTGACTT TGTTCATTCT TCGTTTCCTT CAGGATCTAT GCGATTTGGC |
| 17221 | ACGCTGGTCG CCACGATAAA TTTCGAAACC ACCGCGGTAT TTGAGTTAAT TTGTGAAAAA |
| 17281 | GACACAGGTA ATATTGGTTT AAGAAAAGGG AGTGGAGAGT ATCCAACCGT AAACCGAGTA |
| 17341 | TTGCTTGATT GTGCAAATGC ATTAGGTGTA CAAATGTGGT CGATACACAA CTGTGTGGCT |
| 17401 | AAATACAATA CTTGATCTTC GCTAAACCCA TCAGGGTAAA GCGCACTGTG TTCATTGATA |
| 17461 | ATTTGCGCAA CCGCGCTGCG TACTTGCGCA TAACTTTGCT CATCAATTTC AATTTCATTA |
| 17521 | GGCAAAGGTT CATATGCATA CTCAGACAAT ATCTGCACAA GATACGCTTC GAGTACTTCC |
| 17581 | ATACCTTGAG GCTTAGCCAG CACGTAATTA ATGATGTTTT CAATCATGGT ACCTGGCTCA |
| 17641 | GATACCAGCA CAGTTTCCAA CAAACCGCCG ATATCTGCTG CGACACTGGC AATATGAGCT |
| 17701 | TGGCCTTCTG CAAACCTTTC ACGTTGTGCC TGACCGTAAA AATTCAACCA ACCAAAATAT |
| 17761 | AAGCGTTCAA ATAAACTCAT CTGCTCTCGC GTCAACCAGA CACATTGAGC AGACAGGTGC |
| 17821 | AAAGGCTTAA ACTTGCCTAT TTGACTTTCC ACTAACGCTC TAAACGCGTT ATTTTGTAAA |
| 17881 | CGAGTCGTCC AGTTAGGTAA GACACAATAT AAATTGCCCG ACGAGTCATT CCCCAGTAGA |
| 17941 | TCGCTTTCCA ATAAATAAAA GCTCTCTCTG TTATGGGTCG CTAGCGGCAT CTGGCCATGG |
| 18001 | TGCGAAAAAC CAAGGCTGCG CATCACTAAG CGACTCAGGC TCGCAGTATG CGCTGTTTTT |
| 18061 | GGTTCTTTCG ATAAATAATC AAAACCGCGA CAACGATTTT CTCCCAAAGC GCCAATCTCA |
| 18121 | TTGAGTAATC GACACTTATC AATGTACTGG CGCAGTAATA CTAGACGTAA TAATAGATCC |
| 18181 | TCATGTTCGC TCGGATCATT CTGGCAGGCA CCAATCACAT TGACATAAAA ACCAAATACT |
| 18241 | TCGCGATATT TCAGCAGGTT TGCATTCGGA AGTTTAATGG CAAAACGTGC CAGCAAATGC |
| 18301 | GCGATACTGT CGTTCAAGCG CGTTAACTGA GCTACTGAAA AGTCACTTTC TAATTGCTGC |
| 18361 | TGTAAACCTT GTTGTTGATA CGCGTCTAAT GCCAATTTTA CTAGGTGATT AACGCCACTA |
| 18421 | ATGCCTGTGA CCGCCTGACT CAATTGCGTG TGCGGTAAAG CTTTGACTCT GTGCCAAAAC |
| 18481 | TCATCAAGTT GCCCTTGCGA AAGCGCTTCG CTGGACAACA TTCGATCCAT AATCTGCCCT |
| 18541 | AACCGCTCGA AATGCGTATA TTGAGGCAAA GCCAAAATAT GAGGTAAAAC ATCCAGTTGC |
| 18601 | TTGTGCTGAT CAGCCAATAT TTGATCAAAC AAGTGTAAAT ACCCTTTAAA TTGTAATAAT |
| 18661 | TGTGCTTTTT GATCAGAGTC GATATCTCGG TTTAAAGACT GCTCTGTCAG CGCATAAACT |
| 18721 | TGTGGCAATT CATGCTGCAG TGAACGGTAT TGACTCAAAG GTAAGAATCG ATGTGACTCT |
| 18781 | TGATTTACTT GCTCGGCGAG CCCTGATGTG TGTTCAACGA TGCGCTCGCC GATTGCATAC |
| 18841 | CTAATGGACG CAATTTCATC TTCATCAAGT ACATAACTTT GACCTTCAAT GACTAAAGAA |
| 18901 | ATAGCATCAA AGAATTGTGT ACTCAAAAAT CCTAACTCAG GTGCCAAGTT TACTGAACTA |
| 18961 | GGGTCTAAGT CTTGAACTTC AACTTGCCAG TATTCATAAC CTGGCTGAGC ACCTTGATCT |
| 19021 | ACGATAAATC TAAACTCTTC AATCTGTTCA ATATTCGGGT GAACTCGCAG TGCATCTAAA |
| 19081 | ATATCGGAGC TATAGAGGTG CTTCTTAACC GGTGTGCTCT CTAAATCTTC GTCTAAAATG |
| 19141 | AAGCCATTAC TTAAAGATGG ACCTACGTAG ATATCCTCTA CATACATCCC TTTATCCAAT |
| 19201 | AATTCCTGAC TAGTGTAACG GGTTAAATTA GGCGATATTT CAAGTGCGAC TTTACTCAAA |
| 19261 | ATATCGGCCA TCACGTGTAC AATGTCTTGC GCATCTTTGA GTACCAAATG CATACCCAAT |
| 19321 | TGCACAGGTA CAGACTGATA AAATTCAATC TTACCCAGTT GATGCGTTAT ACAACGAGCT |
| 19381 | GCGGAAAACG TGTGATGGAT TTTTTCTCGC AAGCTATCTT GTGATGCCTG TGCCGATGGT |
| 19441 | GAAGTCAAAA CAATCACATC AAAAATTGTC GCGCCAGTCG CGCGTTCATC GCGAACAGAA |
| 19501 | ATCGTAATGT TCTTTATCTC TGTGATATCA AGCAACAAAC GCCTATAGTC AGCAAGCGTA |
| 19561 | ACGCAATGGC TAAACATCAC TTGCTCCAAG TTTAAAAACT GCGTCGGTGC GAGAGACTCA |
| 19621 | CCAGGCTCAA CCGCGATAAG ATCCTCAACG GTATAATTGA GTTTATAGCT TAAATCCGAT |
| 19681 | AGCACATAAG CCAATACTTC AAGCAAGGTA ATGCCTGGAT CAAATACATT GTGGTCGCTC |
| 19741 | CATGTATCGG GTGCGAGCTT TTGTAGCTGT GCTATCCCCT GTGCTCGCAA ACCATCTAGA |
| 19801 | TCTTGCCCAG GGTTTAGCAC AGCATCTGAA GCAATTTTCA GTGCGTTGGT TTCCATGATT |
| 19861 | TTCCTTTGGA AAGTAAGTCG ATATGGGAGA TGACGCCAAA CCTTCAAGAT TGTGAAACGG |
| 19921 | TTTATTAATT TGGCGTCGCT TTAAATGTAA GTAGCTTATT GCACAACAAA ATTATGCTGA |
| 19981 | ATTTTCCATT TTCCAATCCC TTCAAATACG TCACCATCTA CATTCGTTAA AGAGATTTTA |
| 20041 | TGATTGCGTG TCGGAACTAA AATTTCGCGG CTTTCATTAG GCGAAATTTG AGAGTATTGC |
| 20101 | CTGATCAGAT CATCCATACG TTTACCTCGG ATCACCTGTA CCATGTTGAC CGCTTGATGG |
| 20161 | GCTTCTAACG CCTGTGCTAC ATCCGCCAAA TAGATGGTTT GCGATAACTC AGCATCCGGT |
| 20221 | TTGTTCCATG GGGTTAAGGT ATCCACAATT AAGTCGTTTA GTTCAACTAC GGTGGTCTGA |
| 20281 | ATATCAAATC TTGGGTCAAT CTGAATGATG ATTTCTAGCT GCACTTCAAT ATATGTTGGA |
| 20341 | TCTTCAACTT TAACATTGAG ATATGGCGCA CAGCGGGCTG TCACTTCATC CTGTATTTTA |
| 20401 | CGCATCAAGT AACGAGGTAC TTTGGGTTGT AAAATATTCA CATCATGGTT AAGCGGAATG |
| 20461 | ACAGTGATGT TTACACCATC AGCATTTTGA TAGGCATTCA CAGAGTGTAA TTCAGGGAAA |
| 20521 | GTCTGCAACG TAAAATGCTC ATAATCCCAC CCCGTTTGAA GCCTATCTCT GTGCCTTAAG |
| 20581 | CGCTCACTGA CACGCCGATA TAAGCGACTA TCATCTTCTT TGACCTTGGC ACCAAAGCTA |
| 20641 | TCAAATGGCT GTTCAATCGA CGCGATTAAA GGGTCCGTCA CAGTTAATTT ACTAATGCTC |
| 20701 | CCAGCTGCCA ACGGTTTTAA ATAATGAGAT TGAGCATGTT CAGCTGAACT AAGCGTAATT |
| 20761 | TGGACGGCCT GAGCATAGAC GCCTTTTAGT TTTGAATAGA TAGGGTTGAC ATAGCCATCT |
| 20821 | TGCAGTACCT CTGAATCAAT GACGGCTCTT ATCCAGATCT TACCGTCTTG ATTAAACTGA |
| 20881 | TCCGCTAAAT CAAATTCAGG TAAAGAGAAA ATAATGATCC CCGAATCTAG CAAGTCATAG |
| 20941 | GTATTGTCTT GTAATATTCG CCCTTCTTCA CCGCTGCCTT GAGAGTCTTC TCGACTAAAG |
| 21001 | AGGTGCCATT GCCCTTGATT GTAGTATTCC CACTTAAACT GCGGAGGCTC GGAAAAATTG |
| 21061 | TAGCCATCAA CCGCTTCTAG TTGAAACAGT ACGCTGCATT GGCCAGGTGT TGGAAGATCT |
| 21121 | CCAATAGCTA GATACAAGTA ACCCAAATCG TCTATTTTCG GCATTAAATG AAGCGTATTT |
| 21181 | TGCTGCTCCA ATATTTGCAG CTGTCTGCCA ATAGGTGAAA TATGTTCTAT CCATATAGAG |
| 21241 | TTACCTATGG CAGCCCTTTG CGCCCGAGAA ATACTTTGCG TTCGATAATG TAATTTGACT |
| 21301 | GAGTCTAATA ATGGGGTATA CGGCTCTGGT ACCTGCGTTG GCATAAAATC ACCATCCGCA |
| 21361 | GGATTCCAAT TTGCTAACTT AATACTATTT TGAAACGCAT AATACTCAGA TACCTTAGTA |
| 21421 | TATTGCGGGT GACCAAAATC TTGATTGGAT AAAGCCAAAG TGTACCATTT GGGCCATAAT |
| 21481 | TTGGCTTGGG TCAAATTGAA TGGCAAAGAC AGATAATCCA CATCACCATT GTCGTCATCG |
| 21541 | TAAGTAAAAA CCAAACGGTT AGTCGCGATA TTATTCACAA CATCAGGTAA TTGACCGGTG |
| 21601 | TTGTCTTGCA CTATCAACGT GCGCTCATCG GGTGTCAATT CATTATCAGA CGCTTGCCAT |
| 21661 | ACCAAGTCGT TATAAAATAA GTCCGCAACC AATTTGCTCT GCTCACTGCT ATAAGTCACG |
| 21721 | TCCGATTGCG ATATCAATAC TTGGTTTCTT GGCCAAGCCT CACTCGCAAG TTGAACAGAC |
| 21781 | TCAGTTGGTG GTGTCATTGC ACTCTGATAG TCCATATAAG TTTGGTAATG CGCGTCAAAA |
| 21841 | TCTGCCGGTC TACCCACCCA ATTAAATTCA ATACTTGCCC TTGTCACTCT TTTACTTAAC |
| 21901 | AGCTCAGGGT GCGTGAATTC GAACTTCTCT GCCAGCTGAG GGGTAAAACC AAATGGTTCA |
| 21961 | AAGGGCTTAG TGGTATCTAA AAAGCCAGTA CCATTATTGG CAACCAAGCC ACTTGCACCA |
| 22021 | AGTACGGTCA CCGCCATTCG TACTTCTTGA ATTTCGCTGA CAGCTAAGTT TGCCAGTGCT |
| 22081 | TCCACTCTTT CATTAAGATC CAATGCATAC AGCGCGTCGT ATTTTGACTG CTTTAACACA |
| 22141 | AAGGCGATAT AAGGGAGCAC CATGTCTTTG CCAATCATTA AATCAGCGAC AGGGGCAATG |
| 22201 | GGCGCAAATA GCTCATCAAC ATGAATCACC AGTTCATTCT CACCATTGAG GCTAAGCTGT |
| 22261 | TCTGAGCTTA ACAGCACCGC TTCTTCTTCG GTACTTAACC AAATATCAAA GCTCTCAATC |
| 22321 | CAAAGTGATA AATCAATGGG CGTTTCGCCA AATTCATCGG TAGAAAAACG CAGTGAGATA |
| 22381 | ATGCGTTTAC CACTACTCAA AAAGAGATCC TGAGAAGCAA CTTTAAAGCC AATCTCTACC |
| 22441 | GGTAATTGAG TTTCTGACAT TTGTTTATCG CCAAATGTCA TTGCACTACT TTCAAGCTCA |
| 22501 | ATCCCCAATT CAGTATCCAG TAAGACATTG CGACAGATCT TCACGCCACT TGCAAAGTGC |
| 22561 | GCTTTGTTCA CCGTTGTCAC CGTCTCTACC ACTGCACGAT TTATCGCACT GGGTTCAGCC |
| 22621 | AATTGGTAAA TTCGCTCTTC GCCGTTGTCA TCTTTGCCGC CATCAAACTG AGTGCCAGGT |
| 22681 | TGCAGCGTCA GCGACTCGAC CTCGTTTAGT TCGATTAAAA CATGGGCTGA ATCAGGCGTT |
| 22741 | GCACTTTGCA ACTCAAAGCC CAATACATCG CGATAATAAT GGTCAAGGTG GCGTTGAACA |
| 22801 | AAATCGTTAA ACTGCTGTTG GGTATGCTGT AACAGGTCAA CAAATGTAAG TAGTAAAGAC |
| 22861 | AGGTCTGGTC GGCTCGCCAA TCGGGTCGTA AGTTCATCGC CGCTTCCTAC CAGAATTAAT |
| 22921 | TTGGCCAGTG CGGCAAACTT ACCTGGCTCA GGTAACACTT TGTACCAAGT TTGTACTGGC |
| 22981 | TGCCACGTTG CTTGACCAAT AAATTGGGTG TGTTGCGCGA CAGAAGACAA GTAAGACAGC |
| 23041 | CATTGCTCAA TCGTACGCGG TTCTATACTG AAAAAATCAC TGGAAAGCTC GGGGATCTGT |
| 23101 | CGTGCGGTTT GACTTAACCC TTTACCGTTG CCAGAGATAA GTGATTGACT ACGAGACTTA |
| 23161 | AAAGTCATGA GATTACCTTA TTAATGTTAT TAATGAGCAT ACCTATGCAC TACTAGGTCA |
| 23221 | ATTTAACCTT GGTAAAAGGA TAAGCGGTGC GGGTCTGCAT ATTGCTTGGC GAGCAAGGTC |
| 23281 | GAAATGGGTG ATTCTGTGGG TGATACCAAA TGTTTGAAAC GCGGGTTTAA ACAGACTGCT |
| 23341 | CGGCGAGATA AAACGGAAAC ACCATATTGC TGCGGCTATT GGTTTTGCGG ATCAAATAAG |
| 23401 | TGAGCTCTAT TAATAAGGCA CCTTCGTACA CATCTGACAT ATCAAACGAA ATATCCTCAA |
| 23461 | GGATAATTCT CGGCTCATAG TTTAAAAGCA CCGCAGAAAC TTCTTGCTTA AGCGTGACCA |
| 23521 | GTGCGGACTC ACTGACATCC TCAAAAACAA AATTAGATAA ACCGGAACCC AGCTCGCTCC |
| 23581 | AATAAGGCCG CTCGCCTTTG AGTGTATTGA GCGCAATAGA AATGGCCTGT TTAACCAATG |
| 23641 | TCTCTCCGTC TTCCATATCA GGCCCTGAAT CCGGGCTGGT AAACTGTGGT GGGAATCCCC |
| 23701 | ACCCTCTACC CAGAAATGAA TTGTTCATAG CTACCTCCTC GTTATCCTAT TTCCATCTTG |
| 23761 | TCTGTGGCAA CCACCACTTT TGCACTTTCA ATGGTAATTT TACTGTCCAT TTTTATCTGA |
| 23821 | CTATCATCAG GTGTTTTAAT CACCACATCC GTTTTAGCAG TCATACCCGC CTTGTCTTTT |
| 23881 | TGAGATAAAG CATAACCTGA GCTTTCGCCT AACGAGATTG AGTGGTCTTT ACTTCCTTCC |
| 23941 | ATGATGATGT GTGGTTTTTT AAAGGTGAAC TTCAGCGCGG TGGCTTGTTC TTTAAACAGC |
| 24001 | AACCCTCGAA CATCATATTC CTGTTTGTAT CCACCCAAAG GAGGAATGGC CACTGGGTTG |
| 24061 | TGACATGCCC CAACAATCAC AGGATGTCTG GCATCCCCCC CGATAAAGTC CACCAGCACC |
| 24121 | TCAGTGTCTG GGTTAGGGGG TAAAAATAAC CCTTCTTCCT TGCCAACATA AGTAGTCAAA |
| 24181 | AGCCTTGCCC AAATAGGCTC CGTGGTTAAT GTATTTAAAG TGATGGGTAT GCGATGTAAT |
| 24241 | TTTTCTTTAT CGTCTTTAAA CGGCGCAACG GTAGCAACCA GCATTGGCGT AGCTGGCAAT |
| 24301 | TCAGTCCATT TAGACCAGCC ACTTTTCGTT AAAGATAAAC CCAGAGTAAA TTCACAGAAC |
| 24361 | CAGCCTTGGT TACTTAAATG ATGGTGAATT TCTGTCACCA TGTAGTCCGC TTTGTTGCTT |
| 24421 | TCCCCAGCTC CCGATACTTT GATTGCATCG AGTAGTTTCA GTGTATGCAA AGCGGTGGCT |
| 24481 | TCAGACATAT CAACTTGAAT ACGGCCTTGG CTCGTATCTA ATAAACGATA TACTTGCTCG |
| 24541 | GCTTTGGCCT TGGCAGCCAA CTCGACTTTG TCAATGGGCG CTTGTGATTT GATGACGATA |
| 24601 | TCTGCGGTCG CGCCCTCTTT TTTCTCACCC GAGTCATAGT TTTCAAGTAA CATGGCTTGG |
| 24661 | GTTTTAATAT CCCATGCACT GTGGTCAACT TTTTGCGCAT ATGAGCTATT GTCCATACAA |
| 24721 | AGCTCAAATT CAGTACACCC ATCCATCGCC ACATTAAACT CTGTTGCCGC CGGTTTGTGC |
| 24781 | GTTGTCAAAT CAACGATGTT GATCCCTTCT TCTTCATAAA GCGCAAACCC ATTGGTCAGG |
| 24841 | ATGCGATTCA TGATCACTTC CCACGGTTTT TTCTCAACCA TGAGATATTG ATAGTGTTTT |
| 24901 | ATTTTACTGT CCGCGACGGT TTTTTTACCG CCACAGGGTT TGAGCAAATC ATTGATAATG |
| 24961 | TCGGTGTCAG TAGAGTCTGG CTTGTAGAGC TTACTTATTT GGGACTGACA GAGCTTATTG |
| 25021 | GCTTCACCTT TAGCGAATAC GGTAATATAG GGCTCACTAC AATAACCAAC TTTGCCTCCT |
| 25081 | GTAATGACAC CAACAAATAA TGTCATTTGC GCATCGTCAC CAAAACCGGC TTTGACTTCG |
| 25141 | ATGGACTTAC CGGGCTGAAA ATCTTTATTC AAATCCAGTT TAAAGCTTTC CTCAGCCATA |
| 25201 | TCGCCATCTT CGAATACAAT TGACAACTCA GAAACCTGGT TTAGCCCACG ATGGGTGACG |
| 25261 | ACTTCACGTA CACCGACTTC CAGTTTCTTC TCGCTACCAT CAACTAATAT CGTATAACGA |
| 25321 | ATATCCATAT CATCGCTCCA ATGGTGGGTA GGTAATATAG TCACCCACTT CAGCGCCTCG |
| 25381 | GAGCGAGGGC AATCTATTTA CACTCGCTAC TTGATGAACA TAGTTTGCTT TCCCGTATAT |
| 25441 | GGAAGTAACT TTCGAAACCA AACTGTCCCC ATCTAAAAAT TGTAATTCGT GCGTGAGATC |
| 25501 | TGGCGAGCTT TTACCTGTTC TGAGTTTAAT TTCTTTGCTC GATAGACACT CAGAGATATT |
| 25561 | ACAAACAACC TGAGCTTTGA CTCGATTACC TTTGGAGTTA ACCAACTCAG TAATGACATT |
| 25621 | AATATCGCTG AACATGCCAT GAAAACCGCC ATCGGCACCA TGATTAAGGA CCATATTCAA |
| 25681 | GGGCATAATA GTGACGAACG CAGGCAAGTG CGTTTCCCCT TGTACCGCAA GGCCATATTT |
| 25741 | TAGCATTTCC TGAATACTGT CTTCCGTTGA CGTTTCACCG CTCATGGTCA TTGCATATTG |
| 25801 | AGAGGGGGTT TCAATAATCG TGTTATCAAG TAAAAACGTA ATCGTTAAAA CGGACGGGGG |
| 25861 | GTGATTTTTG ATAACGTGCA GAGCCGCTTT CCGAGCCAAT GGCCTTTGAG TCTTTTAAAC |
| 25921 | AATTTTGATG AGTGACATTG AGTGATTTAG GATCATATGG AAGCGTTAAC TCACCTAATT |
| 25981 | TATTGCCATC GCCACGCTCA ATTTTTTTAT AAAAAGTCAC TTTACATAGT GGTAAGCCAC |
| 26041 | TTGCGTTAAT GCCGTCTGCT TTATTCATAA TTAGGGCCTC TTTTCCTTAC CTTGACGTGC |
| 26101 | AAATCTAAAC TCAATATGTC GTTTTAAATC TGACTCTAAT TGGTGTAATA AGTCTTGCAA |
| 26161 | TTCGGACCAG CTTACTGTGT TGCTATAATA CTCAGACTGT GGTGATTGCT GCGCCTCGTT |
| 26221 | GCCGGACACA CTGGCAGAGT TTTCCACCGT TGATTGGGCA GGTTCGTCAG CATGGATCCG |
| 26281 | TGCTTTAACT CGAACATTGT TACACTTAAC TTCCATCTAT TGAGCTCCTT ACACTGGCAC |
| 26341 | ACCTGGAATA GGCACAGCCA CCAAATCGCG ATAACTAAAA CTAAGAGACT CAATAAGCAC |
| 26401 | ATCATTTCTA CTCGCATCAA TCCCCTCCCA TTCCCAGCTT TCTAAAAATG CATCTTGCAC |
| 26461 | AAGCCAAGCT TGCGATGGTA AGTAGTTATC ATCTAAGGTA GTGATCAAAA TCTGATTTTT |
| 26521 | AACCAAGTAG GTATTCCAAG CATCCCCAAA TACGAGATTT TGCAACATCA AAGGACTGCC |
| 26581 | ACCTTGAAAA ACGCCACGCT TTAAAGTGAG TGTTTTCGCT TGTTTTTGAT TCGCAATGCT |
| 26641 | GACGCGGTTA TTGGTATAGT CGATACTGCG GTTCATTTTT AGACCACTGA CTTCTTTAAA |
| 26701 | CAGAATATCA ATGGGATTAG GAATGCCTGC GGCCACAATA CCAACTAAAA ATCGATACCC |
| 26761 | GACCATGGGA GTTCTAGGGT CTTGAAACAT ATTAGCCACC TAAGCGGGCA CTTTCGTGCC |
| 26821 | CTAAAAGAGA GTTAAAAATT AATGGAACTC AATCTTGATG TCATCTGCCA TCATCTCTAA |
| 26881 | AGACTCAACA CTGGCCTCGT TAGAGCCACC ATTGATACTC GGAGCGGTCA GTTTTTTAGG |
| 26941 | AAATGCATTA ATCACTTTCC ATGTTACCAA TGGCTGAGAC CTGTCTTCAT TGGTCAATGA |
| 27001 | AATTGTGATG TCTTTTTTAT CAACTAAGTT CAAGCTGATA GATGAGATCC AATCGTAGAA |
| 27061 | TTGGCTCTTC TGTTTAACCA GACCACGCTT CAAAGTAATG TTGACGTCTG TTGGTTGCCC |
| 27121 | AGGCATGTGT TTTTTGCCAT AGCCATCTTT ATACGTAATT GTTTCAACGC CGATGTCTAG |
| 27181 | ACCAGATACT TCTGAAAATG GAATGCTCTC TTCGCCGAAA CTGACAACAA ATCGATAGAC |
| 27241 | GGGAATTGGA TATTCTGCTG CGATATCTGC TTTAGTAGTA GCCATGTAAT AACTCCAAAA |
| 27301 | ATTCGGTATT TACAGCTGTG CTGTAGGAAA AATAAATATC TATTTGACGC CATGGTTCAG |
| 27361 | CGCCAAATAG CAATGTTTAA TTAGCTAATT GCCTTTAGGC GCAAGAATTA ACCTTCAAGG |
| 27421 | CTCTTATGAG AGAATGTTAG AACGATGAAT TCAGCTGGGC GCACTGCCGC AAGGCCAATT |
| 27481 | TCAATGTTCA TTAAGCCATT GTTGATGTCA TCTTCAGTCA TTGTTTGACC AAGGCCAACG |
| 27541 | TTGACGAAAA ATGCTTGCTC TGGTGTTTCA CCAAAGAAAG CACCTGAGCG CCAAAGCCCT |
| 27601 | TCTAGATAAC TTTCAATCAT GGTTTTAAGT TTTAGCCATG TGAATGGCGT GTTAGGCTCA |
| 27661 | AATACTGCGA AGTGCGTTGC TTTTTGTACA GACTCTTCAA CCATGTTAAA TAGGCGACGT |
| 27721 | ACAGACACAT AACGCCACTC GTTATCGTTA CCCGCTAGCG TACGAGCACC CCAAACAAGC |
| 27781 | GTTCCTTTAC CGACGAATGT ACGGATAGCA TTGATAGACT TACCAGAAGT CGCATCTACG |
| 27841 | TTGAGGTTTT CTTGATCTGC ATTATCAATC GCCAGTTTAG GCATTAATAC TTGTGCAAGT |
| 27901 | GCAGCATTCG CTGGCGCTTT CCAAACACCA CGATCTTTAT CTGTTTTAGC CATCACACCC |
| 27961 | GCGATTGCTG GTGAAGGTGG TAAATCAAGG TAGTTTTTAC CAAGTTCTGC TTTTACTTGG |
| 28021 | TTATACACAT CTGCTGATGA GAATGGGTAA GCCGGGTCAC CGATTGCAGC AAGCTGCAGC |
| 28081 | TGAGGAAGCA TGATGTCAGT TAATTCGAAA TCATCTGAGA TAGTAATTGC AGAGCCGCCT |
| 28141 | GGTGTCGCTG TTACTTCACC GTCTGCTAGA TCAACTTGCT CACCATCAGT AAGGCGATAA |
| 28201 | TATTTACTAC CGTCTGTTGC AAATACCGTT TCTGGCACTA AGTAACGATT ACCTGATGGG |
| 28261 | TCATATTCAG CATCCGTTGG GTCGTTCGCC CCTGCAGGGT TATAAAGGTA ATCTTGGCCA |
| 28321 | ATTGCAGCCC AAGTGCCGTC GTCACCCACA CAAACCAGTT CATGTGTGTT CGCTTTTGGC |
| 28381 | CACCAAGCTT GCGCTGTTAG TGGAGTATTA ACAACTACTT TCTTCGCGTC ATATGCACGT |
| 28441 | GCCATTGTTG TTGTTAAATA TGGGTAGTAA GCTGCACCAT ATTTAAGGCC CAATGTAGCG |
| 28501 | CCCGCTCTCA ATGCTTTTGA ATCATCCGCG ATTGGGTCAA TGCTGTCTGC ATCCGCTTGC |
| 28561 | ATTTGAACGT CAACCAGTGC AAAGCGATCC ATTCTTTTTT CAGCATGAAC AAGCGCCTTA |
| 28621 | TTTTGCACTT CATAATGCTT AACAGTGCTC AGGCCAATGG CTTCTGGACA AGAGATAAGT |
| 28681 | GTGACTTCAT CTATTTTGTT TAGTAATGTG ATAGCCGCTG TGAACTTTGC TGCTTGCTCA |
| 28741 | GCTAGCAGGT TTATTTCACC AGTCACTGGA TCTGCTGAAA GAAGTCCTGC GTCTGGCGCA |
| 28801 | CCGATACTTA CCACATAACA CGCACCGCCA CCATTGGCAA AGAAGTGACT CACTGCTTGG |
| 28861 | TGTAAAAAGA AATATGCATC CGCAAGCATT GTGCCATCTG ACGTGACATT GAAGCCACCA |
| 28921 | GTTGACTTAG GCGCAACTTT AAAGCTCTCT TGATATGCGC CACCAAATAC AGCTTCAAAT |
| 28981 | TCAACCATGC TCGTAATACG AGTTGGTGAA TTAAGGTAAG CAACATTACC AGCATCATCG |
| 29041 | AGCTTTGTTG TATAACCGAT GAAAGCAGGG ATAGCAGTAG CCACTTCTGC AACTGACGGA |
| 29101 | GGCAGAGTTG ACTTCTCTTG GACGTAGACG TCTGGGGTTT TATATTGAGG CATAGTTTTT |
| 29161 | CCTTACGTTG ATAATTACAT TCTAAAGAGC CGATTAGGCA AGAATTTTGT GGGAAAAGAG |
| 29221 | GCACAAAGCT GTTCGGTCAT AACAGTTCAG GTGGCAAATC AATGGGAACA AACCCACCCT |
| 29281 | TTCCTTTGTG CCTCTGAAAT CGAAGAGCCT TTGCACGGCT CCGCCACATC AGTCCCAACT |
| 29341 | GATGTGTATG TATTAATTAA TTTGTACTGC TATGACGCCA TGGCCTAGAG GGTGTGAAAA |
| 29401 | TTAAAATGAA TATTTTTTAA TTTATTGATT TATAGTATTT TTGACTGAAT ATTTCTCTGT |
| 29461 | GAATTAACCC GCCCCAAGAG CGCTTTACAG ACGAGATAGG GTAAAGTCGG TGTAATCATG |
| 29521 | ATATGAAATG CGAAAACGAC TAAATTGAAT AGAAACGATT TGAAAAACCA GTTAAATATA |
| 29581 | CGCTCATAAG TAGTGCGTAG GCACTATGCA GTGACTCGCA TAAGTCAATT TGAGTGATTG |
| 29641 | ACAAATTCAC TTGATGACAA TTCAGCTGTG CTTGACTAGC AAGTAGATCA AGATATTCTC |
| 29701 | TGTCAAAGCG GTTATTAATG GCTTGCGCCT GTAACCAACT GTCTCTGTTG GTCGCGATAG |
| 29761 | ACACATAATT AAGTTCATAA CTGTCGATGA GTGGTTCACT GAAATTTGCA AAAAGCGCAT |
| 29821 | TTAAAACAAA TCGGTTGCCT CCATAGGCCG TTATGTTTCT GTAGTCTGTG TATTCTACAT |
| 29881 | ATTCACCTTC CTCATTAAGC GCTTCCCAAT GTTCCAACGC CTTTTGCTCG AAGAACCTAT |
| 29941 | GAGGTGACTG ATTTAAGGTT GCTTGCTGCT TTTCATCAAG CATCCTTGCA TGATACTGCA |
| 30001 | GTTCGTAAAA GCAAAGCCGA CATAAAGTTG CACTTCTGGC CAATAGGTAG CCTTGGGCAT |
| 30061 | CAACGTATAA CTCGGCCCCT CTGGGTACCG CAATATTAAT TCCGTAAAAA GCATCACTAT |
| 30121 | AAATATCGAA ACTCAACGAA GATATATCGA GGATAAAATT GCCATAAACT GATTTAAATA |
| 30181 | ATTGGTCAAC CTGATTAAAT TCATTGGGTG TGGCACTACT TAAAGTCAGT TCAGCGAGCG |
| 30241 | TGCGCGATTT CACTTTAACA AATTCAAAGC CCTGATATTT TACCGAGTAT AATGGTGGCA |
| 30301 | ACAACTCATT GTCCGTGTCT AATTCAGAAC CAGCATGCGC TTTAGTTACA AACAAATTTG |
| 30361 | ATAAACGGGG TAATTGCACC ATGTTTCGCT GTCTGTGCTT CGGCTGTCTT TGAGCTTTTG |
| 30421 | TCACTTTATC TAGCGTGAAC AGCTCAGAAG TGCTTTTTAA TCCTTCGGGT AATACCCTCA |
| 30481 | ACTTAGAATT AACCAGCTTA GCTAAATTGG TTGCTGGGAT CACCCAACTG ACATTAGCGG |
| 30541 | CGCCATTTTC TAATCCGCCG TTCCCTATAC CTACCAACCT GCCCTGGCTA TCAAATACTG |
| 30601 | GGGCACCTGA AAAACCAGGT AAAAGACTAC CGTCCAAGTA GTAAATAGGA AACTCCACAT |
| 30661 | CGGGTATATT GGTTTTTGCT AGCGTATCGA CGGCATAAGG TGGTAAAAAC TGCTTTAATA |
| 30721 | CTTCTGGCTT TGCGTACCCT TTCAATAATT CACGTGTACT CATTGCCAGC GCGCCATGGT |
| 30781 | GAAAGCCCAA TGCAGTCACA TGCTCTCGAT ACTCTGGCTT CGCTTGCTTA ATTTGATTTA |
| 30841 | AAGGGCGCCA CCCTTTTGGT GGGTTAACGA CCTCAAGTAA CACTAAATCT GCTTCAGGGA |
| 30901 | AAACACGAGA CACCTTCGCA AGGCGTCTTT TCTTCCCAAA ATCTATGATA ATTTTACTGC |
| 30961 | GTGGGTGCGG GTCCATCACG TGCAAAGACG TCACGACCCA ATGGTTTTTT TGCCACAAAA |
| 31021 | ACCCAGAAGC GACTCCCATT GCATTGTTCG GTTTTTTGAC AACGATTCTG ACTGTACTCT |
| 31081 | TGCTCAGAAC ATCAGGCTCT AAACTGGCAA AGGCATATTT AACGGAAAAA AACGTCAACA |
| 31141 | ACAGTGACAG CAAAAACAGT GACTTTGTCA TACTATCAAC CCTGCTTGAC TGGACAGACC |
| 31201 | ATTTCATGAA CGAACTTCAG CGCATGGAGC TTATTCACCT CTGATAGTCT TTGTTCATTT |
| 31261 | AACTGTTGCA AGGTTTGATG CCAAGCTTGC GACAATATAT GTTGGGCTTC ATCAAACTCA |
| 31321 | GTTATGTTTG CAAGCTCCCT ATCATCTAAG CAAGGTTTTT CATCGCCGAG TTCACCACAA |
| 31381 | ACCGTATCCC GTACCGCCAA TAAAATAGAC TTTTGGTCGC CCGGCTCCAC TTGTCGCACT |
| 31441 | ATCGACTCAC TCAGAGTCGC CCACTCATCG CCAGTGAGGG TAAAATTATT AATCACCTCT |
| 31501 | TCCAGCTCAA GGCTACGATC TGTATATCTT AACTCTTCGC AACCCGTCAC AGATACACTG |
| 31561 | GCACTGAATA GCACGCTATT ATGCTGGCTA GATAACAAGT TTGCGTGCTC TGAAAAGTGA |
| 31621 | AAGGATCCGC TGGATGATGC GCCCTCAGCG CTACTGACAT GCTCTAGTGT CATGCCAAAC |
| 31681 | TCTTTTAAGG TCAAAAGTTG TAGAGCTTGC TGATGTGTGT AACCTGCTGC TAAGTATTGT |
| 31741 | GCTTTAAGTT CCAACAACGA AGGCGTAAAT ACATTGTGGG CACGTACATA AATCCCCATG |
| 31801 | TACGCCCCAA CAACACAGGC AACCCCAAAA GCACCAATAC GCAGCGCCTT AGCATCATTA |
| 31861 | AAGTGACGAT CATTCAGTCC CAAAATAGCA GCCAGTACTG CCGTCAATGC ACCGAGTAAC |
| 31921 | ATTGTCACTG TCGGCGTCAC ACTTGTGCCC ATAATTACAC CGAACAAAAG GCCAATACCT |
| 31981 | GCCCCGCCAA AACAGGCGAT TTTAAGGTTT ACTTTCCTTT CCAATATGAT TTCCTTTGAC |
| 32041 | GCGTTAATTA CCTTATTAAC AGATTAGGGG TTTTATACTT CCGCTTACGA CGCTTGGTAT |
| 32101 | TTTTTCCACC AAACTTATAT TCATATTGTG CCGAGAGTGT ATGTGTGTGT CCGCTATTAC |
| 32161 | TCAGCCATAA CTTTTCGAAT TTGACTGTGA TCAATGTACT TGCATCAAGA CGGTATTCAG |
| 32221 | CCTCTGACAC TAACGCCTGT TGTGATTGAC TTCCCCAGTA GCTCCGCTCA ATATAGTTTG |
| 32281 | GAAAGACAGA GTGCCGGCCA CCCACATACA AGGTTAGAAA ATGCGAAAAT TGATACCCGT |
| 32341 | ACTCAATAAG TAGAGAATGA TAAACCTGAC TTGTGTCGTC TAACGACTCA CTCTCAAAAT |
| 32401 | TCAACTCTCG AAAGCTTAGT ACATAATCTA GATCCACTTT TGCATTTTCA AAACGCTGTT |
| 32461 | GATGACTCAA TAAAATAGCA CCACCTAACT CTTTATCCTT AAAGCGACTT TGTTGATGCT |
| 32521 | GGTATTGTTC AGCACCTGCA TACACATCAA CCCCGAATGC GTACCCATGG GATAAGTCAT |
| 32581 | ATTCGTAAAA TAGTTCGAGC CTAGATTTAT GTCGCCGATA ATTCACTGAG TCAATAAATT |
| 32641 | GCGCTTCGCC AACACGGCTT ATTTGGCCTT TAAGCTTGGT ATGATCTAAC GTTAGCTCCG |
| 32701 | CATAAATAGA TCCACTCGAA AACAACTTGC CACCCGCACT GAAGCGTCCT TCAAGCGTAT |
| 32761 | TGGAATCCCC AGTGAAAACA TCACGACTTA TTGCTTGGTT AACAGGCTCT CTGCGCAAAG |
| 32821 | AAATCGATTG ATTGTATGCC GAGATATTAC CAAAGTAGTT GACCTTTTTA TGCCAAGGCA |
| 32881 | TACTGGTTGA GCGCTTTTCT TTAAGCAAAA CAAGAGGCTG AGCACTCAAG CCCATACTCA |
| 32941 | TCAGCAACAT GCTAAGCAAG GCGAAGACAG TCGATGGTGT TTTTACATCA CGTGGTGGCT |
| 33001 | TTTTATTACC CATTTCCACC GGTCACCCCC GTTACATTCA CAGCCTGAAT GTCTGCTTGT |
| 33061 | AGCTTTATAT TTTCAGCTTT TAATAAATCA AAATTGATCG GTTGGGTTTG TGCCTCATGT |
| 33121 | AACCTGCGAT TGATGTCGTC TGGGATATTT TTCACTAATC CACTTTGCGC TAACTTAACG |
| 33181 | GAGCTTGCCA TCAGTGCCGT TTTTAACGCT GATAACTTAT CTCGGTTCGC TGCATCTTCT |
| 33241 | GGCTGGGTGC TAAATTGCTT AATTGCTGTG ATAAGCGGAG CTAATTGTGC GGTATTTTCG |
| 33301 | GCTACCGCCG CATTGACTAA ACTCAATAGC AACAGAGTCT GTTCTTGCTC AGTACTGAGT |
| 33361 | TGGTTAACAG GCGTTAGCTC AGTAGCCAGT ATATTACGCT GCTGATCAGG CAAATTAAGT |
| 33421 | GCCGTCAAAA CCACTTGACT GGCTTGCTGA GCAATCGAGT CAAACTCGCC TAGGCTTGTG |
| 33481 | CCTTGGTTTA ATTTATTATT GATTTGGCCA TACACCAAAC TCGTTAAAGC ATTTACTTGC |
| 33541 | ACAGAGCCAA TTAAATTATT GGACGAAGCC ATATAATTAA GCTGTAAATG AGCAAGCTCA |
| 33601 | GGGGCTGTGA CGACTTCTTG ATAGTTAGCA ATTAAGCCAC CATCGCTATC AAAGCACTCA |
| 33661 | ACTGCATCAC AAACCATTGT TGTGTCTTCT ATTGCTTTGA CCTCAATGTA AAAAACGCTG |
| 33721 | TTTTCGCTCA CACTGAACTG ACTGTCAAAT GACCCAAATT CATCACTTTG GCCTTGCCAA |
| 33781 | ATGGTTGCAT GTTTTGCATC AGAAATAATA ATAGAGGCAT AAGCCATTGT GCCTTTGAGC |
| 33841 | AAACTGCCAT TTATTAAGAG TTCATTACTG CCTCTTATCA CCCCCTCTTT GTAACACCCC |
| 33901 | ACCGGCATAC ATATCAGCAA AGTTATGCTG AGAAAACGAG ACAACTTAAC ATTATTCATG |
| 33961 | AGATTTCCTT TCATCCCCTC TATAAATGGG CATTTAAGTA TGTGTATAAA ATGAAAAAAG |
| 34021 | TGCAGTAAAT TGACTTTTTA TTTTGAAATA ACAATCGGCT GGGTCGGTTC ATATTTGACC |
| 34081 | GTAAGTTCAG TCGGAGGGCC AGGATCCGGT ACTCCCTGCG GATTAAGTGC GGGGATCACG |
| 34141 | CTCAATGCGC ACACAATATC ACCCTTAAGC ACAATTGGCG CGCCGCCTTT AAATGTCACT |
| 34201 | TCGGATAAAA CAATATCGTT AATTGCACTG AATGTGACCG CTCCACCGGC CGCATAGGCA |
| 34261 | CCATTAATAT AAGGCGACAT ATTAACTTTC TTGGTCAAGA ATTTTTCTAT GTCATCTTGA |
| 34321 | ACGGCTATCT CCCCTTGGCA AATTAGTCCT TTGGAAGCCA CTTCTAAAGG GCCTGCGGTT |
| 34381 | GGAACAACCA AGACGCCTTT ATCTAGCAAC AAGTTAAAAA CGGTACTTTC ATTGAGAATT |
| 34441 | TTTATCATTA TTGATACCTA GCGCAATGCG ATAAACGGGT GTGCTTTAGA GTTTTTATGC |
| 34501 | TTTTACTCCT CACAAAGTCA CTACTCAAAA GCTGATGGAG ATGAACGCCT TTGGGCGACT |
| 34561 | CTAGATACAA CGGCGCTGAG CCTAAGTTGT GAATTTTATT TTCGCAATGG ACTTATCCGT |
| 34621 | TGCGAAGTAA GACTAGCGAC CATACTTTTT GATAATCGAC CAAAGTCCTA ATTACAGTTT |
| 34681 | GAATTCCATC CCATAACTTA GAAGTGTTCG ATTTTCAGCC AAAAGGACCT TTATGTTGAC |
| 34741 | GTCAAATACG TTAAAAACCA TTCTGTTCAG TACAAGCTTA TTGCTCACTT CGCACGTACA |
| 34801 | TGCTGCTCCA AAATCTGAAG AGCCACCACT GCTGTTAATT GGTGCTTCAT TCGCGAATGC |
| 34861 | AAAAATGCCT TATTTCGATA ACCTTGAAGC GCCATTAAAT GGTATAGCAA TCAATTCAGG |
| 34921 | GAAGTACTTA TCACTGGGTA ATGCCTTGAT CAGAGAGCCT CGACTATCTG GGCATCTGAT |
| 34981 | AAACGAAGGC CAAGCTGGTG CGACAACATT TGACAGGCTC ACTTGCTTTC CGGGACCAGA |
| 35041 | ATGTGTGGGC CCAGGCTGGG AAGGATATGA AAAGCTATTT ACCAAGGCTT TAAGCAGAGT |
| 35101 | AACTTCATTT TCCGGTGAAG TCTCTGCAGA GTATATCGTT ATTATTCGCG GTAATGATTG |
| 35161 | TAACCACCCT GATGCATTCG GTATTCCGAT GGCAGACACA TCGGAATGTA CCATTGAGCA |
| 35221 | AATGAACTCG TACATTGATA CATTTGTCAG TGTCGCTAAT CGTGCATTAG ATGCTGGGAT |
| 35281 | CACTCCCATA TTTTCAAAAG CACCGGCCTA TGACGCCATA GATTGGGAAA CATTACGAAG |
| 35341 | CCGATTCAAC TGGCCTTGGA TAATCAGTAA AGAAAATTAC GAGACATTTT CTGAACTCAG |
| 35401 | GCTAAACCGT CTCCGAGCTG AAGTGCCCAA TGCCATTTTT TTGGATATCT GGAAAGGGTT |
| 35461 | TGAGCCCATG GATGACGGAT TGCACCCGAA TAGGAAAACC ATGCAACGCG CGGCTAAGCG |
| 35521 | TATTGCAAAA GCCATCAAAA AACACCGTAA GCACAGCGCA GAATAAATAT TAAACTGTCT |
| 35581 | ATAATATAGG GCCGCCTTCC CCTTCCCTGC GCGTAAAGCG GCCCTCTTCG CCATGATTAT |
| 35641 | TGCTATCTAA TATTGCTTGC GTGCCAATCG GCGTAAATAT TTTTGTTGCA TGGCATCCAC |
| 35701 | TTTAATCGTG TTTTTTACCC AAGTCTCGGC AACACTATTT AACTGATTGA ATGTATTTAT |
| 35761 | ACTTTTCTGA TAGCGGTAAT TCTGTAAAAC CGAATGTCCA ATACCACAAG CTTGCGCTCG |
| 35821 | AGCGACCAGA CTTCTGACCT TATTCTCCCC TTCACCGACA TTGACTAACA CATCAATCAA |
| 35881 | GCCGAGCTCA TACAGCTCTT TGGCACTAAA TCGCTTATCT GAATACATGA CTTCCATCAA |
| 35941 | GGCACTACGC GATGTTTTGT GCTGCAACTT CGCAATTAAA AGGTGCTCTA GGCCATGATT |
| 36001 | AAACAGCGTA GAAGGATACA TAAAGACTGC ACCTTCCTCC GCTACAACAC AATCCGTACA |
| 36061 | TAAGGCAACT TCCATACCTG TACCGTAAGC ACGACCTTGT ATCAATGCGA TACTGGTCAC |
| 36121 | ATTGTGTTTC GCGCCGTGTA AAATTCGCTG CAAAAGCGTC ATATAAGTAA ACGCATAGTC |
| 36181 | TTTGAGCCAA ACGCCATCTT GCTCAGACAC ACAATTTGCT AAGCTACCTA AGTCTCCCCC |
| 36241 | CAAACTGAAT ACACCTGCTT TGTCACTGCT GAGGACCTTA ATTACACGCT TTTGTGTATT |
| 36301 | AGATTCTGGT AAAAAGAAAT GGCTCAGCTG TCTTAATAAC GATTTTGTGA AATAAGGTGC |
| 36361 | CTTATTTAGC TGCATTGTCG CCCAATGCAA GGAGCCAGTG CGCATGATTG CCAAAGGCTC |
| 36421 | ACAGTTATAA ACGGAAGTTG CTCTCACCTG CACGAATTTT TTACCCTTTC AAATTAGATC |
| 36481 | GATAACCCGA CGTACTAGAC TGAGCTTTGT GAACAATTCG TTAAAGTAAT GGCTAGATAA |
| 36541 | AGTCGACACC CTGCCACTGA GCTTCTACTT TTAAAGAAAA CACACGGCCA CATGCAATGA |
| 36601 | AAATTCAACT TTTAAGTTTT ATACTTTTTT CAAGCGCCAG CTTAGCTGAT GCGCAAGTCG |
| 36661 | CTAAGGTCAT GCTTGCTAAA GAGCAAGTCT TGGCCACATC GGGTTCGGTT GAGCGCAGTT |
| 36721 | TGAGCCGTAA GTCCCCTATC TATCGCGCAG ACATACTAAA AACAGGAAAA AATGCCCGAG |
| 36781 | CACAATTTAG GTTTTCTGAT GGTACGATTT TGTCACTGGG TGAACATACT CAATTTATTG |
| 36841 | TCGATCAATT TGAACATGAA ACAGTCTCTG AAGCGCACTT TGAATTTATA CAAGGCGCAT |
| 36901 | TCAGAGTCGT GACAGGTCAA ATCACTCAGG TTACCAATCC CGATTTTAAA ATTAAAACAC |
| 36961 | CGATGGGCTC TATTGGGATC AGAGGCACTG ACTTTTGGGG AGGCAATTTA TATAGCGAAG |
| 37021 | ACACAATCGA TGTTATTTTG CTAGACAGTG AACACCCGCT TGTTGTTGAA AATGAATACG |
| 37081 | GCCGCGTCAC CATATCACCC CCTGGGTTAG GTACAACGCT GACTTTTGGA AAGCCACCAA |
| 37141 | GCAAACCAGA GAAATGGTCA GATAAAAAGT TACAAGATGC GGTAAAAACC ATTCAATAAG |
| 37201 | TTTCTCTCTT GGATGAGCTT TAAGTCACTT CATAAAAATA TAGAAAATAT TTATTCCTTT |
| 37261 | GTCTGGCCAG ACTAATTATT GCACTTTAGA AAAGCACAAT AATTTACATT AAATTAAAAT |
| 37321 | GGTTGATTAT TTTTAAAAAA TGAAGTCATC AGTAAAGCTA ATTGAAGCTT ATGAATTACA |
| 37381 | TATTCAATCA AATTAACTAA TTAATTAAAC CACTTGAAAA AACCATCAAA ACATTGATTT |
| 37441 | ATATACATAT AAATTCTTAA CCCCTACAAT TTAACTTTTA CATATCACCA ATTTGCTGAT |
| 37501 | GTTCATTTTG TTTGATACTT TGCATCTCGT TAACACTTTG GGCCCAAGTG ATAACTCACT |
| 37561 | AAAACTAACA AGTAATAAAC AACTGAATTA AAGAACGACC AAAGGAAAAA AATGAAAATG |
| 37621 | TTTAAATTAG GTACACTTGC AGCTGCTTTA TTAACCACAG TAAGTGCCGT TGCTGCGCCT |
| 37681 | ATCAACGTTG CTGATACAAA TGGCTATGAC CGCCACACTG TTTACTCACA CGGCCCAGTA |
| 37741 | AGCCGCGTAG TGATCACCAG TGAGTTTGCG CACAACTTCT CAGCTGACAT TGAACTTCGC |
| 37801 | GACGGCGCTC GTTGTGATAA TGGTAACCTA TATTCAAACG TTGACAGCAG CCATGTTGTA |
| 37861 | AATGTTGGTG TTCAAGCAAA TGCGTCTAAC GCAAATGCAG CAACAATCGG TGTGACTAAC |
| 37921 | GCAATTGCAG AAGACAAATT AACACACAAC AAAACACTTC ACTTAAGCTG CTATGATGCA |
| 37981 | GACAATACTT GGTATAACGT TCTGGTAAAC GTACCTGGTG CGCCTATTGT AAATTGGGAC |
| 38041 | ATTACAGTTG AGCCTGCAGG TGAGTTCGTA AACCAGCCAT ATTCATTTGG TTACCATTCA |
| 38101 | GCATTCCGTG TTAAGAGCAC GCTTAATGTG AACAACCAGA ATAAGAGCCA GTCATACTGC |
| 38161 | TACACAGTTG CTAACCGTGG TCTATCTCTA GGCCTATTCC ACGGTAGTGA TACTTCGAAT |
| 38221 | ACTTTCCATT CAGATGTATT CACGCAAGAC AAAGTGTACA GCAATGACAC TGCACAACCT |
| 38281 | GTTCTTTACC AAATTGTTCA GTGTGAAAAT GCTGCTGGTA AAACGATGGC TGTTAAAGTA |
| 38341 | TTCAACTTAA CAGATCCAAA CGGTATCTAC ACTTATGAAG ACCAACTTAT CGTTAAGTAA |
| 38401 | GTAGTGACGA GAAACGATTT GAAAATGGCA GCTTAAATTA GCTGCCATTT GTTTTGATAT |
| 38461 | CTTAAAGTTG AGAATGGTTA AACGTGGATA CGGAAATAAA TACACGGTGG CCAAGCGCTT |
| 38521 | TTTTATCAGG GCGCTGATAA ACATAACGTC GATATTCAGA AAGGCTTGCT GGTGCCCCAT |
| 38581 | CAAAGCCAGA CAACATTACA CGAGAATGGT CGGGTCTATT CTGCGCAGGC AAAAAGGTCT |
| 38641 | CATCACCAAA ACTAAAACTC GGTAGTTCAG AAACCAAAGC ACCAGAGACG TGCCATTTTA |
| 38701 | TTGGGTACTT GCTCGTATCA AAACTAAATT CCCATGCTGC GCTCTGAATA CGTGTATCTA |
| 38761 | ACCCAGTAAA GTCATAACTT ACTGCATCCC CCTGCTCTAG AGAAGAAATA GCCACAGTTA |
| 38821 | ATTGCTGTGC CAGCTCAGTA GATGCAATTG GAAACGTCAA TTCGCCAATT TGGTCTACTT |
| 38881 | TATCAAAGCG CAGTGTCACG CCATTTGATA TAGCGACAGG GAGCCCCTCA TAATGGAACA |
| 38941 | ATATACTCTG GTAGTTACTG AGTTCATGTT CAGCAAACCC TGTTGGGTAC ATTTTCAGAT |
| 39001 | TGACACCACC TTCAACTGAA AAATCAAAAT CTTGAAAAAA GTGATTGCCT ATCTTATCAA |
| 39061 | CAGCACTCAA ATAGACTTTC GATTTTTGCT CAAATTGTGA CGTAGATAAA TCTGCACTGT |
| 39121 | GGTGCACAAA GTCACTGCCA TTTACTGTGA TTGTATTTAC ACCTTCGGGC AAATCAATTA |
| 39181 | CCGCTAGTAG GCTAGAACCA TAGCCAGGTT GGTCGATAGT ATAATATACC TTTTCGTCAT |
| 39241 | TTCCACAGAC CTTAACAACA TCTTGATAGT CTTGTATATC ACCACCGTTA GATCTTGAAA |
| 39301 | CACGAAACCT GCTATGTGTT ACTTTAAGTG CACTCAGATC AACGTCTATT TCTTTACATT |
| 39361 | TACAATAAGC AGACAGGTCC TGAAATTCGA TTGTCGTAAA ATCAGCGCCT GAAGAAATAT |
| 39421 | CTAACTTGGT ATATATTTTT CGAAAATTTT CAAAGCCACC GTTATTCTCC ATGCCAATAA |
| 39481 | TAGAGGCATG TTTAGCGCCT GCGGGAATTT GCTGACTAAA ATACCCGCTA CTATCACTCG |
| 39541 | AATGTGACGC AACAACTTGA CCGTTGTCGT CGTGAAATAC AATACTCGCA TTAGGGTAAG |
| 39601 | CAACCAAACC GCACTCTGTG CGCTTTTTAA CATTTAATGT CAGAGTTGGC TGTGCATCTG |
| 39661 | GCTTTGTTAC AACAGGATTA TTGTCTTGTG GATCACTTGA AGACCCACCG CATCCTGCCA |
| 39721 | GCGCAAATAT CGAAGCCAAA ATAGGGGCAA CTGTCAATTG GTTTACTTTC ATGAAGTTCA |
| 39781 | TAACTAAAAA AATTTTTCGA ATTGTACACG AAATGCAACT CGTTTGTATT TTATTTAGAG |
| 39841 | ACAAAAATAG AATGCAAGCT CATGTTTGTC AGGATCTTAA GTCGTTTACA TACCTACTGC |
| 39901 | CACATTAATG AGAACATAAT AAGATGTAGT AAGTACAAAT TTAACCGAGC TGCTGCCGTG |
| 39961 | ATTACATATT AAATCACTCG ATTTAAGCAA GCGGAATTAA TTGAACCAAA CAAGGTATAA |
| 40021 | TTGGGTTGGG AAAATACGCT CCACAACTTC TCCATTTTCG CCTTTGCTTA AATTTAAGAA |
| 40081 | AAGATAAAGT AAAAATTAAC TTTCTTGTAT ATAATTAATT AGTTAAAAAT TAAACCCACA |
| 40141 | TTTAATTAAA GAACAACCAG ACAGCCTAAA ACCTCTTAAC CAAATTCAGT TAAAATAAGA |
| 40201 | ATTAAATATG AATAAACTTG CAAGAATAAC TATTCTAACT TTCTGCCTAA TCGCACTACA |
| 40261 | AGGCTGCTAT TTTTTTAGTG AAAATGTAAT TTTAGAAAAT TGCTTTTCTC CATTTTCAAT |
| 40321 | TTCTAAGCAA GCACTTTCAA AAGACGTCGA GACCCGTATC TTACCTCCGA GTAGCAACTT |
| 40381 | AATTGATGAT GGCTATCGCT TTACTCTAAT TGATAAGTCT TACGATTATG GCAAATATAG |
| 40441 | ACAATCTACA GGGAATATTA ACTTTACAGG GAGCTTAATA TTAAGTGTAC CAAATTGGGC |
| 40501 | TCACAAATAT ATTGGTGGAA ACCCAAAAGG TTTATACTTT GAAATAAAAA ACGGTGGGCA |
| 40561 | AAAAGGTATA ACTAATTTTT TAACAATGGA CTACTATATG CCCAACCACC TAGTTAGTAT |
| 40621 | TAAACCGCAC TGTAAGGATG AGCTGATAAA AACGTGTCCA AACCAAAAAT GCAGCTATAA |
| 40681 | AAATATATTA AATTAAAAGT AAACCTCTTT TTTGGGGTTA TAAAAAAAGC AGCCATCAGG |
| 40741 | CTGCTTTTAT TGAATCAAAG AGCGAAGGGC CATCTCTGAC CGCGTGTGTG TATCGCCAAT |
| 40801 | TAGGCTTTGG CATGGATCAA TGTTCCTTTT ACATGTTGTA TCGATACGAG CAGTTATGAA |
| 40861 | CTCACAGCCA CTCAAATCTA ACTAGCAACA TAAAGAACTC ACTAGCCAAT ATCTATTACG |
| 40921 | CCTTATTACA CTCATTGTCA TTTTTTCAAT TATCAGAACA AGTTAGTTTC TGACTAACAG |
| 40981 | ATCATTACAT TAAAATGAGC CTTTCTTTTT CAATACTCTC TAAAAACTCC CCGGCAGCTT |
| 41041 | CTTTATTTTC TGCGTTTTTC GAGCGGTTTT TGCGATAGTC CGAGGCTATC TCTTCTAGCT |
| 41101 | TTGCAAGTAA CAGTCTCCGC TCTAAAATAG AGTGCGTTTT GTGATATTTA TCCAGCTTAT |
| 41161 | CCGCCACTTT TGCTTTACTC TTACTCATTT CAAAAATCCA GAAACTCTTG CGCTGTACCG |
| 41221 | CTTCATTCCA AACTTTAGAG GTCCAAATAT AAGTGCCACT GGTCGGTAAC TTATCCCTAT |
| 41281 | CAGCTTTGGT TTCTCGATAA ATTTCAGTGA ACTTATTTTG CTGCGATATC AACTTGAGCA |
| 41341 | AATGAGTATA TCGCTCACTT ACATCCTCTT TTTGCATTGG ACTCAGGTGA CTATATACCG |
| 41401 | TTTTACGTGC AACCTCAGGT AACTTTCTTT TGGCTTCATC TTCGGTGTAT TCAATATGAG |
| 41461 | TATGATCTTT TTCTAACTGA TTAACCCACT TATCTACTGG GAGTGCCTCC TTAGTTACCT |
| 41521 | CGGCAGGTTT TGGTTTGCCT TCGGTAAACT CCCCTTTAGA GCGCTCAACG ATATACCCTT |
| 41581 | TCCCATTAGG ATCTTTTTTG ATTTTACCAT TAAGCGTATA CTCACCCAGT GTCCAATCAC |
| 41641 | CAATAGATAC CGACGCAATT TCTTTTTCAA ATGTCAGTAC TTTGGCTTTT ATCGATGCAC |
| 41701 | CTACTTCCGC TTCTAATGAG CCGGTTAGGC TAAAGCTCGC CTCCATTGGA TAATCACTAT |
| 41761 | CAATGGCAAA TTGACCAAAG CTATCACCGC CTTTGCCACG CGTGTATTTC AAACGCCCTT |
| 41821 | CTGTTTCAAC CTTACCTTCA ACACCACCTC GGATCTCACC AAATACACCA CCTTGAACCT |
| 41881 | TTGCAAGATA AGGGACCCCC ACAAAAGCAC CAGCCTCAAT TCTCGCTTTA GCTTTGGCAC |
| 41941 | TGGCCGAAGC TGCACCATTC ACAGAGAAAA GCATTTGTGT CTCTGTGTTA TGCTGCTCTT |
| 42001 | CATTTTTAAG CTTAGCGCCG ATGCTCGCAC CAAACTCAAA CCCCCCGGTA ATATCAACTC |
| 42061 | CAACTCCTGC AACACCAGCC GCAACGGGAA AGTCCAAAGA GAAAGCCGGA AAATCAATCC |
| 42121 | CTTTGTGCCA TTCTCCAGTG ATCCCACCTT CACGATCTTT TCTATTATAG TAAGCCTCAA |
| 42181 | GGCCTAAAAT ACGTGCTCTA AGTAACTGTA AACCATTGTT GCTATCCTTC TCGTAGGAGA |
| 42241 | ATTTACCACC AGCAAATTTA AGCGCGTTTA ACCCTTGGTG GACTTTCAAC TGCTTAAGCT |
| 42301 | CAAACACTTT TCCAAAATCG AAGTTTCCAG AGAAAAAGTC TGACTCGACA ATGGCCGTTT |
| 42361 | CATTAAGCAC TTTGGCATTA TCATGATCGA GATCCATAAA AGCATTTTCA GCTTCAATTG |
| 42421 | CATCTTTTGT AAACTTAGCC TTCGTCATGG CCAGCGTCAT TGTAGTGTGT TTGCTAATTG |
| 42481 | GGATCTCCAC ATCAGCCTGC TCAACCTCAC CGCCTAATTG CTCATCGTGA TACATCACTT |
| 42541 | TTGCTTGCGT AATTGATGCT TTCACACCAG CGGGTAAGCC GTAAAGCGCA GCGTCAGTAC |
| 42601 | TCACCTCAAA CTCTGGTGAA GCATCCTCTT TAACTTCTAC TTCACCACTC GGGTTGCTGA |
| 42661 | TAGACAAAAA CTTGGGGATT ACTTCGAATG TATCTTTAGA AGAGATTTTG GTCCTGCCTG |
| 42721 | ATTTGAATTT ACCTGCAGTA TCCAATTTTA AATCAAACTC ACCATCCAGT TTCTTTTTAT |
| 42781 | CCCAAAGCGG CACATGTGCA TCACCTGCAA AATGAACTTC TTTTTGCTGC TTATTGTAAT |
| 42841 | CAAGTGACAT TAAATCGGAG TAAACCCGCT TGTTCGCAAT GTTTAGCCCA ACAATGCTTG |
| 42901 | CTTTAAAACG AGCAAGGCCT TCTTCATTAA TGGTCAGCTC ATCGCCCAAT ACTTGATGCG |
| 42961 | CTTCAATATA ACCACTAAAA CCCACCCCTT GTTGGCTTGC AAAAACCGGA GATACAGCCA |
| 43021 | CTTCCCAGCC ACTGCCAAAT GGTAATTTTA CATCTATATC ACCACCAAAT TGCTGACCAA |
| 43081 | GCAGATTGAC CTTGGCAACA CCCGTACCAT GTAGTCCAGC GCGCTTGGGC TTACTTGCCC |
| 43141 | CCTCTTCCCA GTCCAATAAT TCAGGTTGGT TTATCCCCAA CGAAATGAAC TTATTATCAA |
| 43201 | CTATATTAGG TGATGTTTGT GCATCTTGAT CCGTTTGTTG CTCATCGCTT TGCTCATCTT |
| 43261 | CACTACTTGA ATCCGAGACA CCAATTAAAC GTGCAACGCT ACTGGCAAGC TTATCTTGAA |
| 43321 | TATACTTAAC CCAAGCTTTT AAATCATCCA CGAGTGCCCG TAATGCCCCA TGTGCATCAC |
| 43381 | TGCCTTTTTT TAACCATTGA TAAATATATG GCTCATCAAT ATCTCCCCAC GCAGCGGCTA |
| 43441 | TCCAACCACC GTTATAAAAT TTATTTGCAG CCCAAAGCCA GATTTTTCCA ATGGCCCATA |
| 43501 | AAACGGCGGA AACAACACCA GGCGGAACTT TTGCCAAAAG TAAAAGTGCT TGTGAGATTG |
| 43561 | CCGTCACCCA TGGGTTCAAG GACTTTAATG ATGTCACCGC ATAATTAAAT ACACTTGAGA |
| 43621 | TAATCAGCTT AGCTGTTGCA TCGCCATTAA AATGATCCCA AACCACTTGT GAGGCATACC |
| 43681 | GCTTTGCAAC ATCAACGCCC GATGAGAGTA GATCGACACT CACAGAATCA GAGTCTAATG |
| 43741 | CATTGCTCAC TGATAACGCA GTGCGCGTGG GATAACTTTG AAAGGTAGGA GCTTGCTCCT |
| 43801 | GTTCTTGTTG TTGCATTTCA ATAATTTCCT ATTGTTGCTT ATGTAGAGAA TAGAAACTTA |
| 43861 | GAAGAGGTGT TGAGTAGAAG CGCAAATGCA TGTGTGAAGC GTGTATTTCT AATATCAGTG |
| 43921 | AGAGTTAACT TAGCCTTTTA GTGCCGCTTT AAGCGCATCA ATTCGTGTCT GGTCCCATGT |
| 43981 | CCAAAACTTG CTATTTTTTT GCTTATCTGT CCAATCTTTA TAGTCTTTAT CTCGAATGTG |
| 44041 | TTTATCATCT TTATGTGCAA GACCCAGCGG CTTAGTAAGC CCTTTTCTCG TTGTCCAAGG |
| 44101 | TCGCATATAT GCCTTGCTGT CAACCGTGTT GTCTTTGCTT TTAACCGCTT TGTTTGTAAA |
| 44161 | TGCCTTTAAA GTCGACACAT CTCCTGTAGA GGCATGTTTG GTTTTGTATT CAGACGTGAT |
| 44221 | GGTCACTGAA AACGACCCTT CAGGGTGGTA ACTACTCACA GCATCCGTAC TGATATCACT |
| 44281 | TAAGACTTTT ACCTCGCATA CTAAATTAGC CTTAGGCGCA GCTCCGCTCT TGTACCCAGG |
| 44341 | GGCGGTTTCT GTTTGATGAG GTGCGGGGAC TGTTGGTACT ACTTCTACTT TATATTCAAC |
| 44401 | AACATAACCG GCTTCAACGA GGTTTTTTAC ATGAGATTCA ACTCGAGCGT GATGCTCTTT |
| 44461 | ATTCGCAGAT CCTGTAATAG GATACAAATT AAATTTCAAT GCTGAGCCGC CTAAATTGTC |
| 44521 | GTTGAGAAGG TGGCCACGGA TCCAGCCTTT TTCATTGGCA TTATTCCCGT TGTTATTGTA |
| 44581 | AACAAGCTTA TTCTTCATCA AATCGCTTTG ATCGGCATTG ACTTCAGCAG ACTCGCCTCT |
| 44641 | GGGGGCTAGA CGAGATAATC GAGCCTCAAC TTTATCACCA ACGATATTAC TAATCGTTAC |
| 44701 | ATCAGCACCG GATGCATTTT TATACTTAAA ATCTTTGGCT GCAAAAAACT TAATTTTTGT |
| 44761 | ATCATTTATC GAATCAGAAC CAGCAGTCTC ACGTACCCTT TCTTCGCTGC CACCAGCAGG |
| 44821 | TATGGCAATA TTCCATGCTG GGTTAATTGA GAATGAAGTC CCTATTAAAT ACGGGTTGTA |
| 44881 | TTGATAAGCA AAGTTTTGAA TTAAGTTAAC TCCAGGCATG TCTTTTTGAG CCACTTGATT |
| 44941 | GATGGCTTTG AACGCAAAGT CAACATCCTT GCTCTCTGGC AGAAGCAGTT TGTAAATGTG |
| 45001 | GCTAAGCCTC GCGCCAACCA CTTGATAGTA CCGTTGTGCG ACATCATAAA CTTGCTCTTC |
| 45061 | ATCCATCAAC ACATCAAAAT AGCTGCTACC TATGACTTCA ACTGCTTGTT CAACTCTCTC |
| 45121 | AGTCGACGTC AAATCAGTCG AGTACTGGTC TAATATAGGA ATAAAGTTGT CATACTCATC |
| 45181 | TTCGGAAATC GACTCACTGT AATCTAAATC ATCCAGCACA ATTGTCTCAA TATCTTCTTC |
| 45241 | TTGCATACTC ATATCTGAGT TTTGCCGAGA TAGGTTATGC TTTGGCTGCC GAATTGGGCT |
| 45301 | GCGTTCTCGA AAACGAGAAG GCGCTTCTTC CGTTCGATTT CTTTTTTGAC GACGAGCTTT |
| 45361 | GTTTCTTTGC TTCTGCTCTT CGCTATCAAG CAAGTATGAG GCGTCCATAT CTGACATCAT |
| 45421 | AGTGCGCGGT CCTTTAGTAG TTTACGAATA TTTGAGCTCT GCTGAATAAG GCTTTTCTGA |
| 45481 | ATAAATAAAA CGGAGGGTAT TGTGCTCATT GACTTTAAGC ATGGGTGCTT ACCAATGAAA |
| 45541 | ATGTATTGTC TAGATCGACT CGAAGCAGCT TAAAATGAGT GCTTGCTTCA CTTTCTAATT |
| 45601 | CACAAGTGCT AATTGATAAA ATACTTCTTG CTCAGCCCCT TCGCCTATGG CCTCTTCCAA |
| 45661 | CGCGTTAAAT CCAACTTGCG TTAAAATGTG CCGAGAGGCC ATGTTATGTA CCCAAGCAGA |
| 45721 | GGTCTTAATA TGGGAAATAT TCAGTTTGAG CGAGCGCTGT TTTAATGTGG CTACTGCCAA |
| 45781 | GCTAACCCCT ATTTTTCCAA AACCACGATG CTGGTAATCG CACCCCACCC AAAATGATAA |
| 45841 | GTGTGCATGA CGTTGCGAAT TTTGATTGTC AATCGCATCT GGCAGCGGAT AAAAATCCAC |
| 45901 | AACAATTGCC CCCACAAAAC CAAAAGACTC ATGAACGAGT GCAAAATGCG CCTTTTCACC |
| 45961 | ACGCAGCCCC TCTTCAAGCC AAGTCGGCCA AACTGCTTGT AATTGATTAA ACTGTTCAAT |
| 46021 | TTTCAAACCA CGCAGGCGTT CGGCAATATC ATCTTGTCGA TACTGAATAT AAAACTCACC |
| 46081 | TAACTGATGC GCCCCAAGTG GTAAAATTCT GAGCCCTGAC ACGTCGCATC GAGTGCACCG |
| 46141 | AAGTCCTTTA TCAAACCAAG ATAGTTGGTT AGTTTGCTGC TGATAAGTAG CAATATCGAT |
| 46201 | ATTAAGCTGC TTTACTTGCG CATCTTCAGG GCTGAAATCA AGTGCCAATT CTGCGCTTTG |
| 46261 | ACTCGCCACT TCTTGCTCTC CAGTAGCCCA AGCCACCAGT GCCAAATTAT GTAAATATGC |
| 46321 | TGTACTAGGC CCATTAACTT CCATACAAGC CAGCATACAG TTTTTCGCTA ATCCCCAATG |
| 46381 | GCTAAGATCA ATGGCCAACA ATCCTAGTGC AAAAGCGAAA GACTCTTGTC TACAGTCAAC |
| 46441 | AATGTGATTT TGCCAAACTT GAGAGAGTAC ACCGCACCAC TGTATACGAT TTTCAACAGA |
| 46501 | CACCCCCTGC TTTAGCAATT GTGGGAGGAA AATCTGCAAC ATATTCGGGT CATATTGACA |
| 46561 | GAGTCTAACA TACGCTAACA TTTGCGCCTC TGTTAAGCTC TGAGTGCAGC TTTTTAAGCT |
| 46621 | TTCATAGATA TTTACCTGGC AACTGGGTAA ATGCTCAGAG CAGGCTTTCT CAAATATATG |
| 46681 | CTGCGTGTAA ATAAATTCCT CTGGGTTTTG ATGGCCTAAC CTCATACACG TCGCCATCTC |
| 46741 | TGTGCCTTCG TGTTTTTTTA TTTGGGTATG GGTTACAGCC GAACTCACAT AACTTAATGC |
| 46801 | CTTCAAATCT AAAGGTAACT CCACCCCAGT TTGTGTAATC ACTGTGGGTA GATTAAGCCC |
| 46861 | GAGCTCTGGC GTCAAATGAT CGCTAATTAA ATGACATACC CCTTTATCAA AAGTCTGCTC |
| 46921 | TAAACGCATA CATACTTGTA CCGCATTGAT TGGAAGCGCG ATAGGTTTAC AGCCCCCGCT |
| 46981 | TGCCAACTCT TTTCTCAATA TGTCGTCAAA TGGCAAAGCT AAGTCGCCGT GCCCGACTTC |
| 47041 | AAGATCAATA CTCGTCTTGC ACCACTGATA TGCAAGTCGA ATTTCATCCT CTGCTTTATC |
| 47101 | CGAGGGTGAG TGCGCTTTCA TACGCCAAAG CAGCGGCAGC TCTTGGTTAT TAGCCGCTTC |
| 47161 | AGTGCTTGCA GCACTGTTGC TGGGACCGAT ATTTGCTGTA GTTGCTATCT CAGCTCGGTA |
| 47221 | AAGGTCTTGG TAGTGTGTGT GATATATCGC CTGTGGCAAC TTAGAAAGCA CACCATGAGC |
| 47281 | TATCATAAAT GCTGGATTTT CGCCGAATGC AAGCATCGTT TCGTCAATAT CTTGATGCGG |
| 47341 | TAACGGTTGT TCTGAAAAGA TACTCCAATC TAGTATGTGT GCTTTTCCAA TCTGATGATA |
| 47401 | ATGGCTGAAA TCGGGGTGGC ACAAGAGCTG TGCCTGTGCC GTTTTATCTG TTTCCATCAA |
| 47461 | CAACAAACAC ACATTGAATT CTTCCAAGCC TTGCTGTGGT CCCTCTTCCA AAAGTTTATT |
| 47521 | CATTAACGCC AAACCAAAAG CGTAACGCGC CTCTCCAAGG CACAATAAAT ATATTGGCTC |
| 47581 | AGACTGGTTT AACAGATTAT TGCCTTTAAT GTCGTGTAGC CAAGCTACAA GCAGATCAAA |
| 47641 | ATAAAGGGCG ATCCAAGCTT GATTATGCCA GCAATCGTTT TGTGGTGACG GGTCTTGTGC |
| 47701 | TGACCAGATA TCAGCCTCTG GCGCTTTGTT AAATACACAC TTTTGTTTTT GTTGTTCAGT |
| 47761 | CAAAAAAGTC GCTTTGGGTA CTGGCTTTAA TTGCGCATAA CCGACGCCAT TAAACACCTC |
| 47821 | ATTAAGCTTT TTTTCGCGTG CTTCTTCTGT CATGTATATG CCCTGTTTTT TGGTTTGTTA |
| 47881 | ACGTCACGGC AGTTTAATTA TATAGTGAAG TTGGATAGCT CCACCTGGCG GGGCATTTAG |
| 47941 | CAGTGGTCTT CCATCTTTAC CATCGCATAA CCCGTAACCT AGTGGTAGTG AATGACCGTG |
| 48001 | GTATAGAAAA ACTGCCCCAC TTGGCACCCC ATTTGTCTGG GTTGCTTTTG TTTTTAGCGA |
| 48061 | TGTCGTCTGT GTGTGTTCAG CAAGCCAAAG TGTGCCCTCC ACAATTAAAC TGCCACTGAT |
| 48121 | CTTCAAGTTT TGAGTGATTT CTAGGCCCGT GCTGCGCTTT TCAATACCAT CTTCACCTTG |
| 48181 | CAATAAACAT GCATCTAGCC ATTGGGCAAA CTGTGACTCT GTCGGCTTTG CGCCGCGCTT |
| 48241 | AAAAAAAGAT TTAAGCGTTT TTCGAAGCTG CATTGCCATT TAAACTCCTT GCATGAGGTT |
| 48301 | AAGAAAGTTT AAGATCGCTT GCAGTGCTGA TCTCATTTTT AGGAAGGTGA AGTGCTTGCC |
| 48361 | AAACCTGGCC GGTATGAGAT ATTTCAGTAA CTTTAAAGTA ACTTTCAGCG CTCTCATTAT |
| 48421 | GTGCTGTATT TACTGATACT ATGTTACCTA GCTCCAGCTC AATAGCTGCA CGTTGCCATA |
| 48481 | GACCAGATTC ACTTATCACA TATATGCCAT TCTCTGTTTT ATCTCTTTGT GCTGTGAGTA |
| 48541 | ATACATACAT TTGCCAATAA CTTGGTGGTA GAGGTAAATG TATATTCTGA TCAGTATTGA |
| 48601 | AAACCACATC TAAGACGCCA ATGAACCCAA CAAATCGTTC TATATGTCGT TGGTGCCTCA |
| 48661 | TAAATCTATC TAGTAATTGA TTAACATTGA CGTACCCTTC AGCCATATTC ATAATCCCTC |
| 48721 | GTAAATAACT GGCCAAGCAC ATCGCTTGAC CAGTTCATAA TTAAACTGCG ATCGCGCCGT |
| 48781 | CACTTAAGGT GACTTCACTC CACTGATCGC CTTCCATCAA TTTATAAAAC ACAGTAGATG |
| 48841 | AAGTCGGCGG TGTACCAAGT AACTCAACTA AGTTACCTAG CTCAATGTTT GGATACGTCA |
| 48901 | CTTTGTTAAG TGTGTCAGCA ATTCGTTGAT ACACACCATT TTGAGAAGGA GTAGATTGCT |
| 48961 | TGGTAAGCAA AATCTTGTCG CCATCGGCAA GATTCAACGT GTTGGTTAAA TAGTTGTCAT |
| 49021 | TGTTTGCTGT CGACAAATCG ACATTCGCAT CTTTTTTAGC GCTATTCACA CGGTCAATAA |
| 49081 | TACCGGTATA ACGATAAAGT GATTTCGTTG CTGTCATATT CACTGCAATT TGTTCAGCAT |
| 49141 | CAATCGCTGC GAGAATACCA GAGAGCGTTT TTTGAGCCAT AAGGCCTCCT TGATAAATGT |
| 49201 | TGCGTGTACA TACACACGCA ACGGGTTAGA AAATCGAATT AGCTATCGGG AACAGTCCTA |
| 49261 | ACTGGACGGC GTAATGGTGA AGTACCTGTG ATGAGTTAAA AATGAAACTT AAGGGATATA |
| 49321 | TTCAAAAAGA CGATAATCTG CCACTTGATA GTGCACAAAC GCACTGGTGC CATTGTCATT |
| 49381 | ACCTATGCTT GTGATAATGC CAACGACTCC ACCTTCGAAT ACCACAGTAC CTCCAACAAA |
| 49441 | ATCGGTAATC GCTAAATCAA TTTGATTATC AGTAAACCAG TTACGACTAC CAATGTTATT |
| 49501 | AAAACCCGGG TTGCTATTGT GGTGGGTATG GGCAGAGATA GATGAAACAC TCGGCTTATC |
| 49561 | TTTAAGGTAA TTTTTTTCCT CTTTGCCCTT TGTATATAGT GCTAACATGC GTCGACCGGC |
| 49621 | AGGTGCACCA TGTTGTGATA ACGTTTGCTC GGCAACTATA TATTGCCTTT CATCTAATAT |
| 49681 | TTTGGTTGTA TCCCACAAAC TGTTTTCTAC TAAGTTACCT AAATAAGTAA CTACACTTTG |
| 49741 | AGATATTAAG AAGCGTTCTT TACTTTGCGG GTTATATACT TTTCGAACGT AGCCAAGGTG |
| 49801 | ACTTTGAATA CTGCCCGACA CGCTTTGCCA AGCACTGCCT ATATATTCCC AAACATCGTT |
| 49861 | ATTTTGTGCG TCATATAGTT TTGATACATC TAAGCCGCCC CGTAAATCGA TGTCAAGATA |
| 49921 | AGTTGAATTA GGCTGAGAGT AAGTGATGTA GCCATAGGAT TGCTTTCCCT TGAAGCGGTG |
| 49981 | CAAATCAGTA TCATTATTTA AATCAACCAC AATTTCACTA GCCGACTGAA AGCTGCCAGT |
| 50041 | TTGATTCCAA GGGGTCACAT AGAATTTAAA AATATCGCCT TGACGCTGAA CCTTTACACG |
| 50101 | AACCCGTTTA TTGCTCCAAC TACCACCGGT TCCAATATTA TACTGCCCTA AAAGCTTATC |
| 50161 | AGACCCAGAG CCCACCCCAG CATAACCAAA ATATACACCA CACCCTGTCG CGTTGGGAGG |
| 50221 | AGTACCACCT GTATTCAACA CAATAGTGAG TACATAATTT ATGTTTCCTT CTCTGACAAA |
| 50281 | TGCGGCAACC ACACCAATGG TATCATTGTC AGTACTATCA GAGGATAACG TCGCTTCTAG |
| 50341 | TGTATAGTTA TCGACTTTTT CTGCGGATAC AAACCCGTTT GAAGGTGAAA CATTAAGTGG |
| 50401 | CATCACGACA CTATCTGTAG AAGCTTGGTA AAACCAGGCT TTTGCGTTAG CACTATTGCG |
| 50461 | AGCTTCAGCG TCTTGTTTAT TTAAGTAGTA CTCATTGCCG TTAAATCTCG CCCAATTGTT |
| 50521 | GAATATATCT TGTACCGTCG GAGGTCTGTA ATCTTGTCGA GCTTGATCCG CTTGTTGTTG |
| 50581 | AGTACGGTAT ATATATGATT CAAGCTCAGG CAATGGCGTG ATATTGATGT AGACGACACC |
| 50641 | CGTTTGCTCA ACACCATGGC CGTTACGCAC TTTATAGTTA AATTGCGCAG GTTGTTCTGC |
| 50701 | AAGACCCGTA GACAGAAAGG TAATTGTATC GCCACTCAGA GATACAGTTC CGCCCTGCGC |
| 50761 | ACTCGATACA CCGATTAATG ACAAGCCAGT ACCAGAACCA TCTTCATCAT TGGCAATGAG |
| 50821 | TTGCTGCTTA CTGATAAATA CAGACTCACC CTGCTGCAAA CTGAACGTAT CAGGGTTACA |
| 50881 | CACGATAGGT GGAACAGCAA CGACTGACAT TGTGACGAAG TGTGTTTTTC GGATCCCAAT |
| 50941 | ACTATTTTCT ACCACATAAT TAAAACCTGC CGCGCTCCCG ATCCCACTAT TTGAAGTAAA |
| 51001 | CTCAATGTTA GCTCCCTCTA TACGCACTGT GCCAAACAGA GGGGTATGTA CGGCTATCAA |
| 51061 | TCGCACTGGA TCTTGCCCGT TGCTATATTC ATCAAAGCTC CCTTCGAGGA TATTAGCAAT |
| 51121 | CGGAATGATT GCCGTGCGCT GCGTATAAAC TTCAAAGGTT TTCACCTCTA GGCGTATCGT |
| 51181 | TTGCGCAGAT TTTCCATAAA ACTCGCTAAT TGCAATCGTG TCTCCCGGTA CTTTTGTGAC |
| 51241 | GTGCTCACTA TAAAAACCAA GCTCATAAGG CTGAGCATCA CGGTATTCAT CACCGATATC |
| 51301 | TTTAAGTGAT ATTGGTCCAC TTGTTTGTAA AGTCATTATG CTTCACTCCC CTTAGTTAGA |
| 51361 | CTCAAATTTC ACTATTTTTT GCTCAAGGCC CACCACTTTC TCATTGAGCT CTTTTATGCT |
| 51421 | TTCAACTAAC AACCCCACTA AATTCCCATA AGCCACAGAC ATATATTCAT CTTGCTCATA |
| 51481 | GACAGCTTCG GGTATGACTT GCTCAACATT TTGTGCGATC AGTCCGGTAT AACGTCTACC |
| 51541 | AGTATTAACA TCTGCTCGTT CAAATGTAAC ACCTTCCAGT TTGTGAATCG CAGCCAACGC |
| 51601 | ATTTTCAATC GGTTTGATAT TGGATTTCAG GCGAGCATCA GAGAATGCAG TAACGTCTCC |
| 51661 | TGTTGCTGTT ATCGCTCCTG TGACAGCCAT ATCGCCCGAG AATGTCGCTT GGGTAATGTC |
| 51721 | GACAGAACCT ACTAACTGAG TATTGACCGA TTTCAAAAAG CGTTGATCTG ACTCTGTTTT |
| 51781 | AGTATAACGA TTTGTTGAGT CCGCAGTTCG GTCGGTAACT TCTTGCTGCA AAGCATTCTG |
| 51841 | TAACGTAGCA ACTTGTTGGC TTCTCGTATT AGACTCATTC GCTAACTCTG TTTCTATCTG |
| 51901 | AGCGCTTCGG GCATCATTTG TTAAAACGTA ATTGGCAAAA GCTTGATCCG ACTCTGTGTC |
| 51961 | GACAGAGTTG ATGAGATCAA CAATTTCTTT AAACGAGTCA GCATCGGCAT CACTGGCTTC |
| 52021 | TAAAATACTG TCAATACGCT CCTTTTGAAC CGTAATTTTA GTGTTGTAAT CTGCTTTTAA |
| 52081 | AGCATCCGAG CCGGAAGATT GCTGCGTTTT AAATTGCGCA GCATCCAATA ATTTATCAGC |
| 52141 | CAAACCCGCC TCGATATCAG CTGGGGTCGC AACATCGACA GAAATTTGGT TGTTGCGTGC |
| 52201 | TTTTAAACCA TCGCCAACAA GTGCAACCTG CAACGAGTCT TGCCAGTGAT TTTCACTTTT |
| 52261 | ACCAGAGTCA TTGGTAAATT GAGACGTCAA TACAAATACT TTATGTTGAT GGGAAGTACC |
| 52321 | TGCTGAGATA CATATAGCAA GACCCTGATA TAATGAAATG GCCGCAGCAG GCAACGCCAA |
| 52381 | AGATTTATCT AATTGAACTT GCCAAATTTG ATTTTCTGAT GGATCTTCTT GTGCGGTTAA |
| 52441 | TAAAATCAGA TCGCCTGCCT GAAGTGCAAT ATCATCCAGA GTATTAAGTG GTTGCGAGAC |
| 52501 | ATCAATATTA ACCGACGTTT CAGCGCAAGT GATCGTCACT TCATTGAATG TATACACACC |
| 52561 | CAGCGACTCT ACTTTTTGAT TTAACCACTG ACCGTGATCT GCACTGAGTA TTTTATCCGT |
| 52621 | GCCGCCAGAT TTTAAATCAT TTGTATCGGC GAAATTTAAT CTTTGAGGCG GCACTTGCCC |
| 52681 | AACGGTCAAT TGCCCTGCAT CTAAATCGGT CAACGAGCCG CCATGTCCGG TTATACTTTT |
| 52741 | TGCAGTGATA CTTGAATCAC TTGCCACTTG TGTTAAATCG ATATGAGCTG GCAGGTGTGC |
| 52801 | ATTATTCAGA TTTGTGATGC CTTCACCATC CCCAACCAAA TAGTCTGCAG TCAACTTACT |
| 52861 | ATTTGTCGTG TCTCCTTGCT GAGTTAAATC AACTTCAACA GGAAAGCGGG CGTTTTGAAT |
| 52921 | ATGTTCTGAG TAATACTCAG GTACGTCAAA GTTTTCAACT TTGTACCAAT TCAAGTTTTG |
| 52981 | ACTGGTTTCG TTCAGCATGA CATAGTGAAA GTAGCTGGCA CTGTTATCCA ATGACCTATT |
| 53041 | TGCCTTAATT AACATGCCTT CATATTCATT AATGGTTTGC TTGGTCAACG TCGCGGATCT |
| 53101 | TTCTACCGAA GATGCATCGT AAATGTAGAC ACCATTTTGA GTCTCGTCTT GCTGAAGATA |
| 53161 | TATGATCACT CGCTCACCAG GTACTAGTGA GTATGGTGAT AAAATTTCAG TCAAATCTGA |
| 53221 | TTGCAAGTCA AGGTTGGACT CACTGATTGC GCAATCTACT GTAGTACCGA ATAAATGGGC |
| 53281 | CAGTAAGCTA AATGTCGTCG TATTGTCCGT TGCCGCATCT ATGAGTTTAC GAAATTCCTC |
| 53341 | TTGTGTAGGA ATGTCTCCAT TTTCAAAATA GCCGCGTAAC TCCGTATTCA GTTCTGAATG |
| 53401 | TTTCATAGTT AACCTTTGAT AATTAGTCGT GTATTAAGGG GACTTGCTAC TGGAAGGGAG |
| 53461 | GAAGTTCAGT AGCAAGAAAA TATTGCGGGT AAACCAAGCT TGGATTAAAC CGATTTGTTA |
| 53521 | TCCATTTTCG TTTCAATATC TTGAACAATC GGTAATATAT CTGGCGTGAA TACTGGCACA |
| 53581 | GGTACTGACA AAGACAGCAA CAGTGTAGGC TTGGGTGTGG ATCCTAATGC CTGCCAGACA |
| 53641 | TGCCCACTTA CTTTTTCGCT ATGTTCATTA CCGAACAACT GCGTTCGAAT GCCATAAGGC |
| 53701 | TTATCTTCCA CGCCATACAT CGTAAGAATG TCTTCTGGCA AAAAGTCATA AGCCCCTAAA |
| 53761 | CCACACAGTA AGTAACTCAA CACCATATGC TCTAACTCAG CACTTCCCAT TTCCGCTTTA |
| 53821 | CTCCAGACCG TCAGCATATA AGTTAATGAT ACAAATCGAG GCTCTTTATA ATGCACTCTA |
| 53881 | TGAGTCTGAG CTGTATTTAG ATAGCTTCTC GGTGGCTCAC TTTGTCTGCG TGTCGTGTCC |
| 53941 | TCTGCTACAC CAATTAGATA GCAGTTTACT GTCGGCTCAT CACTTACCTG TTTGTCATAA |
| 54001 | AAAGTCTTGT CGGGCGCGAC AAAACTCAAT GCAATATCCC CTTCTAACTC AGTATCTCCG |
| 54061 | CCTTTGCTCA TAGACAGAAT CCGCTCAGTC AAAAACGTCT TCAGTGCTTG TTGAGTGTTA |
| 54121 | TAAACTATTT TTGGGTCCAT CTTAATTTCC TTGCAACCAA GCTTTAATTT CATGCTGTGC |
| 54181 | CATAAACCCT TCATGCTGTT TCGCAAGCTC TCTTTTGAGC GCTCTGGCTA ATGATTCCGG |
| 54241 | GTCAATACAG CTATGCTGTT CTATCAAACG TTGAAGTAAC GCTGACTCTG TAATATTGAT |
| 54301 | AATTTGTGCT GCACTTAGCT CAAACCTGTG CGCCAAACTG CCAATAGATT TGGTCAACGC |
| 54361 | ATCGTTTGAA ATACCGCTCA ACATACGCTG CCATATCTCT TCCCGCTGCT GAGCTGAAGG |
| 54421 | CATAGTAAAC TCAATCACAT TATGAAAACG CCTCAAAAAC GCGTCATCTA AGTTTGATTT |
| 54481 | TAAGTTGGTG CTTAACAGTA ATAACCCACT GTAGTGCTCC ATTTTCTGTA AGAGGTAACT |
| 54541 | GACCCCCATG TTGGCATTTT TGTCCTGACT GGATTCAACA GCACTACGTT TTGCAAACAC |
| 54601 | AGCATCCGCC TCATCAAACA TCAACACTGC ATTGTGTTTT TGAGCTTGGT CGAATAGCTT |
| 54661 | CGCTAGGTGC TTTTCAGTTT CACCAATCCA TTTACTGGCA ATATTAGCTA AATTAACGAC |
| 54721 | ATATAAAGGA AGCTGTAGTT CACCTGCAAT AGCTTCAGCA GCCATTGACT TACCTGTACC |
| 54781 | AGGGCGGCCC CAAAAAATGG CCTTGCAACC TGGTGTAAAG CGCTGAAGTA AGGATTGTAA |
| 54841 | TTCGGCTTGT CTATCAATTC GGCCGACTAA TTCATAGAGT TGACTGTGTA CTGCTGGAGA |
| 54901 | CAACACCATG TCAGACAACT TGAACCTTGG TTCAGAAAGT TTCGCTAGTT CTTCAGGTCC |
| 54961 | TTTATTTAGC TCTTCCAAAC ACTGCTGCTG TAATTCACGC CAAAAATCAG CGTTATTCTG |
| 55021 | GGGGGTAACT TTAGCGGTTT GCGCTAACCC TGACATACGA TAAATTGGTA CCGGGTACCT |
| 55081 | CGTGGCGATA CACCGTGCTT TTGCTTCATC AGGCTCTAAA GACAATTTCA GCCAGGCGCT |
| 55141 | TACTAACGAC TTATGTGAAG GCGGTTGACA CTCAATCACA TGAAACAAGC TTGTATCTCG |
| 55201 | ATGCGTGTTT GGGGCCTTTA AAGTAAAAAA TACAACCGGG TTTGAGCACC CTGTCCACAC |
| 55261 | TCCAGCATTT TTCGCAGTGC ACAAGCATAT GTAGCATGAA GTTTTTCAAT AAATATAAAC |
| 55321 | ACGCATTTGG AGTTCGCATT TAATATTAAC CCAACCAACG ACAGTACGAT TTCTGACAAG |
| 55381 | GTCAGATCTT GCGCTTGCTC ATCTAAAAAA TAACCAAAAT CCGCCCCTGA TAACAAAGCA |
| 55441 | AGTTGCTCTG TGTACCACTG AGCCATCCGA GGGTCAGGTG TATCAAGTTC AAATAACTGC |
| 55501 | GAATCGCTTA AGTCAATTTT AGGATTAAAA CACGATGTAA AAGCCTCATC CTGAACCGTA |
| 55561 | GACGAGCCCA GCTTTACAAG GTGTTCATTA CTCAAAGTGA CTTGACCGGT ATGCAAAAAC |
| 55621 | TGCCTGAGCT CTGTATGTAA ACTGGCACTT TCAGTCAGCA ACTTTTTTTC AGAGCATTGT |
| 55681 | AATAAATGCC AATCGAATAC TTGGCCACAC AATACATCTT GCGAAATTAA CTCTCGTTTA |
| 55741 | GACCCCCGCT GGCAAAGAAA AAGCAACTTA TCGAGACTTA ACATTGGCCC TTGCTCATAC |
| 55801 | CAGCTAAGAC CGATGTAAGG CATCAAAATA TCCGGCTCAA GAGTTTGAAT ATAAACAAGA |
| 55861 | GCAATCAACC TAGTTTCATG TGATGTGAGA GAAAAACGCC CTTCAACATA CTTAAATCTA |
| 55921 | GGGGTGTTAA CCAGCGTATT TAATGCGTCG TCAAGTTGAC TTAATCGTTC CTGTATATTG |
| 55981 | TCATCATCAT TTGCTAAAGC GATACCAACC TGGAAAAAGC GCAATGATAA CTGCTTTTCG |
| 56041 | GCTTTAAATT GTTTTGCTGA GTCGTCACGC ATTACCATTT TATCACCCAT GTTTCATTTG |
| 56101 | AATGGCGAGC TCCATAGCTC TATCACAACA CGGATCTACC CAGCCGCAGT GCCAAACTTC |
| 56161 | ATGCGCAGCT AATGAAAAGT CCTCAACACA CAAGGCCGCT AGTAGCTTTT TTAACCCTTT |
| 56221 | TAAGCCATCG ATACCAATGC TAAACGCCAT ATTAAGCAGG ACCAGTTTTC TAGACTCACT |
| 56281 | CAATCGATTA AAAATTGGTA CCTCTACTGA CAGAGTTCGA GCTAAATAGG CAATATCGTT |
| 56341 | TTCAAGCATA TATTCTGCCT CTTGCTCGCT AATACCTACC TGCTCTATTT GCCTGCCGAT |
| 56401 | GCCAACAACA AGTGCTCCGT TGCTGTTTCT CACCGGTTTA ACAGTAAGTC CTACATGGCA |
| 56461 | AATCAATTGT ACCTTCAATG CTTCAAGGCT TTGACGAGAC ACACAGTGAG TCATGACACC |
| 56521 | TCCTTAGAAA GCAAAAAATC TGAAGGCACA GATATCATCA AGCTTTGCTC CAGTCCTTCA |
| 56581 | ATCTCGATAA CAAATACGGC GTCATTTTTG CGCTCAACAA GTTTTCCTTG GTAGCCTTTA |
| 56641 | AGGGCACCCA CAGTGATTTC TACAGCACTC CCAACGGCAA ATCGAGTGGG TACTGGTTCG |
| 56701 | CACTGGTAAC CTGACGACAT CACCGTTTTA ATTTTTACAA TTTCTGCATT ACTCACCATG |
| 56761 | CTGGGCTTAC CGTTAAATTT AATAAAGTCA ACGAAGCCAC TCAATCGCTT TACATGGTGA |
| 56821 | TATTCAAAAT CATCGACATA AACAAATACA TAAGATTTAA ATAACGGTTT CGCTATTGAT |
| 56881 | TTAATTCGGT CGCTCCACTG TTTTTTCTCG ACTACTAATG GTAAAAAAAC CTCACAGCCA |
| 56941 | CTCTTTATAA GTGAAACCTG CTCCGCGAAT TTTTTTTCCG TATTCGGTTT GGTGTAAACT |
| 57001 | ACATACCAGT TTCTTATTGT TGTCATCACA AATCCCAGAT ACGCTCCGCC ACATCACCCC |
| 57061 | CAGTATGAAG TAACTTAAAC GGCAGTTAGT TTTTAGCTAT AGCAATAAAA ACTTGTCAAT |
| 57121 | TTCCTATTGC TGGTTTAGTA CCAAGCAGAC GGAAATAGTT ATTTTTTGTG AAAATTAAAT |
| 57181 | TTTTAATAGT TTGAAAAAAG TGACCTTACA TAATAAGGCC ACTTTACAGG TTGGGCTTAT |
| 57241 | GTTCAAAAAC CTATCAAGGT ATAAACGTAA GACTGAGATA TTTGTGAATT TATTTATTGA |
| 57301 | GCTTTTCGGT TAAATATTTA AATTAAAAAA TTTATACGCC ACCAGACTAT CAAAAGTCCG |
| 57361 | TTAGCTATAT CTCTTATATT CTTCTTTTAG ACTTGTCAGC TGCCTTCTCG CGAGCTGCAC |
| 57421 | ACGTTTTCTA ACATTTTCTA GTGATAATTG AAGGCGCCTG GCGATTTCAG GGTAATCCAT |
| 57481 | TTCATATATA AATTTATATT GCATCACAAG ACGTAAATCC CTTGGCAAAA TAGAGATCTC |
| 57541 | ATTTACTAAC CGTGTATACA AGCTAGAATT CCAATGATCG CTTTCTAGTG AAGTAGATTG |
| 57601 | ATTATCTGCG AAGAAAAAAT GGTCCGGTAA TTCAGATACA TGATTAACAA TATCTTGGTG |
| 57661 | TTTTGCTTTA GCGCGATGTT CGTCCATGCA GAGATTGTGA GCGATGCGAC ATAACCATGC |
| 57721 | GAATTCATTC TCGATAGTAT AATCAGCACG ATTGTATGCT CTAAATGCCT TTTCACAGGT |
| 57781 | TTGTGCCAGT ACATCTTCTA CCTTGTCGCT GTCATTTCTT AACCACCGAT GACAACAACT |
| 57841 | TAATAATTTA TCTTTGTTTT CAAGCCAAAC TTTCCAAAAT TCACTATGTC CTTTTAGTTG |
| 57901 | CCGTTGTTTT AAGGCAACCA ACTCCCCTGT ATGTAACATC ATTTCTCCAA TAAAAAAATT |
| 57961 | TATATCGGAG GAAAGACGCG CTATTACCAT TTATGTGAAA ACAAAATTAA TACAATATAT |
| 58021 | AAACCATTGT TTAATTGATG TATAACCCTA TTCTGTTTAA CACTCAGTAT TTCAAATGAG |
| 58081 | AAGCAAAGCG CGTTGTAATG ATAAAACTTT ACAATAAAAT CGGCATTTAG ATTGAGGTTG |
| 58141 | GATAGAATAC CTAATCCAAT TGTGGGAGCG CTTACTCATA TCCAAAGTAA CACTATATGT |
| 58201 | GAAGCGCTTT TGACATTTAA GTGTTCAGCG CCGCTATCTC ACGTTCAATA GCTGGTGTGA |
| 58261 | TCAACAACTC TACCTCACCC CTATGAGCAT ATGAGATACG CAGTCGCCCT TGGTATGTAC |
| 58321 | TTGCAACTAA CGCCAGACCA GCAGGCGGCA ATGGTACACT AAAGTTATAC ACATCAATAA |
| 58381 | TCTTAAAAGA GCCAATATAG CTTAAGTTAA ATTCCAATTT ACCTAGGTTG GATAAGATCA |
| 58441 | CATTACCATT GAGAAACAAT CTTTTATAAC TCTCAACAAG TAATTTAAGC GCACATTTTG |
| 58501 | GTACTAGATT TATTGAGTAA GACTGAATCC TCTGCTCGGC TTGATGCACC TTATACCCTT |
| 58561 | CAAGTTTCAA CTCTTGCAAA CGCGCCTGGT ACGCCTTAAT CCAACAACTT TCGCCTCCGA |
| 58621 | TCTCTGAACC GAATGGCATA ATTAAATTAT TATATGAGCC AGCAAGATCC TTTTTAGCTA |
| 58681 | TTCCCCTAAG CGAAATAGTT TCAAGTACAG TGTATTTTTG ACCATTATTT AGTGCTTTAT |
| 58741 | TTAAGACATA ATTAAGCGCG ACATTCACGG AAACCTGATG CCGTTTAGAC CAAATTTTCA |
| 58801 | GTGCTTTAGT CGCGTCCAAA CCGAAGTCAT GATAATCAAC ACGCCAACAC GCTGAGTCTG |
| 58861 | CAATCTCAAT CTTTGTTTTC GATTTAAATA AATCAATGGC CAATCTAATA CCAGCATGAA |
| 58921 | ACAATGCTTT TGTACCAACC TTATCAAAAA GCATATGCTC TTCACAAAAG CTATTATCGA |
| 58981 | TACTCGAAAT AGGCTGGTTA AGTAACAGTG AATGTAACTG TTCAAATAAA ATGTAGCCGC |
| 59041 | TACGTGCATC TGAGGCCGTA TGTGAGGAAA GAAATATCAA TGCGCTGTAC TGTTTAAACC |
| 59101 | GGATACAAAC AAATTGAACT GGCGATCCAA CGTATGGATC AACTGGGTTG TCAATCGCGT |
| 59161 | ACTGTCGCAA TGCGACATGC CATTGCTTGA CGTCTAGAAA GCGATCGGTC CAATCCAGTT |
| 59221 | GTGACTTTTC AGGGTAAAGC GTCTCATCAT ACAGCCAAAA ATACTTGAGT CCTTTTCTGA |
| 59281 | CAATACGACT TTGCATTATT GGGTGTAAGC TTGCAATTTT ATCCACACAG TTAGACAACT |
| 59341 | CGTCCATGCC ATAATGGCCT TCTAAAATGA CAAGGTCGCA AGAATTAGCA TTTTTATTAT |
| 59401 | TTGCTTCGGC TAAAGCTAAA AAGTTGAGTG CTCGATCACT CAAAGGTAAT AGGTTCATTT |
| 59461 | ATTAAGTTCC TGTTTAACAC ACAAAAACTT TAAACGAGAA TCATTTACTT GTCTAATCTG |
| 59521 | TTATCCTAAT TAGCAGCACA AACATGATTA ATCGCCTACA AATTATTACC AGAACTAACT |
| 59581 | CAAGTTTTGT GACAACTTTT GTTCCAGCGC AATAAAATCG TCACGTAATT TCAATAATAT |
| 59641 | GAAGTCAAAT TAGGTGTATC GTTAAGGATT AAAAATGGCA TCATTGAATT TACATCGCGT |
| 59701 | CTATATTCCA ACTAATGCTC GCAATAACCA CTACATTCTG GCTGAGTTTA AACCTGATGA |
| 59761 | CTCGTTTTAC AGCCACTTTG ATGACTTAGA AAGTGCATAT CAAAGGCTTG CGAGAAAGTT |
| 59821 | ATTTGCACTG TGTGATGAAT ATGAACTCTA TAACGTTCAG CTTATCGTGA ACGACAAGTT |
| 59881 | GCCTGTTGTC CGATATCATG AAGAAGCATA TAGCTTACAA ACAGATAAAC AAATACTGTT |
| 59941 | TTTTACAACC CCAAATACCA TGAAGCCCAT AAAATTTAAT CAAGACGAGG GCCACAAAGC |
| 60001 | GAGAAAGATC CGTTTATTAT TTTTAGTCAA CGGGTGATGA ATTAAGAGCA AATGCAGCAG |
| 60061 | CATTTCACAG CAAAGTAAAG CGCACATTAG ATGCCTTACA AACACAATAC GAAAAAGAGA |
| 60121 | ACATGCGCTT TAAAGTAAGG GACCATCAAC ACCTTACATA CGATATATTC TCGAAAATAA |
| 60181 | AAGGACACCG AGAAACATAT GGCTATAAGT TAAGAAGCTT ATATCCCAGA TACCAGGCAA |
| 60241 | GAAACTGCTC ACTGCCAGAG GCACACAGTG AAATCACTTA TGTTACTTTT TCAGTACCTA |
| 60301 | TCACCAGAGC GATAAAAACT GAATATCAAC ACTTATTGAG ACCAGGGGAT TATTCTGGGT |
| 60361 | TTTACCGACA TATTGAAGAC AAACTATTGA CGACCTGTAC TCAGCTACAG CTTTCTCATG |
| 60421 | TTGGGTTTGT CGCTGATGGT AGAATGCCAA TCATTAGAAA CAGTCAAATT GATAAGTCGG |
| 60481 | CACACAATAG AGAGCTACAA AAGCTAAGCT TTGATACATC TTTAGCAGAT GGTCAAACCC |
| 60541 | ATACAATTTG GGACGCACAA CATTTATGTG ATGTCATGCA TTTTGTCATC GTGGCCAGTG |
| 60601 | ATGCGGATAA CAAAGATGCT GGCTATGGTA AATTTATGAA TAATGTAGAA ACTATGGTTC |
| 60661 | GACGATTTAT TACCCAGCTA CCTATAAACC CTGAGAAACA GGATGTGACA ATGCGGTTCT |
| 60721 | TCCAGCATAT TAGTTATACT TACTAATCTC ATTAGGCAGT CTAAAGCGGG TGGTCAAAAT |
| 60781 | GCAACCACCT GCCCTTTTGC AGTACGTTTA ACATGGATTA AACGACTCTC TAATTCAGCA |
| 60841 | AAGATATTAC CAAAGTCCCC TTCCTCAGAG CATATCAGCG CCCCTGACAT TTGCAGTGTT |
| 60901 | GTCACATTTT CCTGTGTGGC AAATACATGC TGTAATACCG CTAATCGTTT TTGTAAAACT |
| 60961 | GGCTTTTTCA TCCCTTCACA CAAAATGACA AATTCTTGCC CACTCACTCG ACATACAAGA |
| 61021 | TCGCCATCAT CCCTAAACTG CATTGATAGC TGCTGCGCTG CAAATTTAAT TGACTTAGCA |
| 61081 | TCCATATCAG AGACAGTCAT GGCCTCACTT TGTTCAGGAC TGATCTCAAT AAGCGCCATA |
| 61141 | GCATAGTCGA GATTCTTCTT TGAGATCCCC TCAAACTGCT CAATAAAATA CCGCCTGTTA |
| 61201 | TATAGCCCTG TCACAGTATC TTTATAGTTC AAAGACTGCG CTAAATGGTG TTTGGAACGC |
| 61261 | CTAAAGTAAA CAATCACAAC GAACAAAATA ACAATAAGCA GCGCTATGAT AATGCGAGCA |
| 61321 | TGCATTAGCG CTTTGACAAC TTCGTCTTCA GCGGCCGTGT TAATTTGGCG CGCTGATCTG |
| 61381 | ATTGGTTGCT CGAATACAAC TGCTTTGTGT TCAGGTTTGC TTTGAATTTT TAGTTCACTT |
| 61441 | TCTTCAAGCA AGCGAATGTA GGTGCGTAGC GTGGAAATTG TTTGCGGCAA ATCTTGTTTA |
| 61501 | GATTGATAAA CATCGGCTTC AATTTTTAAA GTTTGTCGAG TATAGCGATT ATTAAGCGTA |
| 61561 | CTATTATACG ACTTTAACAC CTGCTTAGAT AACGCGATTT GCTCTAACGC TTGATCGAAT |
| 61621 | AACTTCATCT GAGCAAACGC AAAGGCTAAA TTATTATGTA ACACAATGAT GTGAGCAGGA |
| 61681 | TTAATAATGG CGTTATGTTG TGTTTGTCGA GCAGCTTGCA GATATTTAAT CGCATCCGTA |
| 61741 | AAATTTTTAT TTGAGAGCGC TATTTTACCG AGCCCAGATA AGGCCCAAAA CTCGTATCTA |
| 61801 | GGCAAGTTAT GTAACTCAGA GACGTTATAC ATTTTCTGGT AACACTGCTG GGCTTTTTCA |
| 61861 | ATGAGTTGAG CACGTAACAA CGTTAAGCAC AAATTATACT TAATAGGGAG ACCGTCCAAC |
| 61921 | AAATTGTGAG ACTGCTGAGC CAAATCCTGT GCCTGATGTG CATAACCCAA CGCTTTAACA |
| 61981 | TAAAACTTTA GCGTCGAATA GTAGGCTGTG GAGCTATCAT ACACCATCAT TCTCGTAATG |
| 62041 | GGATCGGACT TATTTTCGCT CCGCGTGAGC AGCGCTTGAG CTGATTGAAG TGATTTGAGC |
| 62101 | GCAAAACTGT ATTCCTCACG CCAAACAGAA ATAATCGCCT CCATTGCATA GGTTCGGATC |
| 62161 | AGAAAATCGG TTGCTTTATT TTGTGTAAAG CACAACCTAG CTTGTTTGAT ATATTGCTTT |
| 62221 | GCCTTATCTA ACTCTCCCTG CTCTATGGAA AAAAAAGCAC GATACAAGAA CCAATATCCA |
| 62281 | GACGATAGCT TTAAAGGAGA TGCTTGAACT TGCACTTTTG CCGAATCATA AACAGCCTCA |
| 62341 | GCCGTTTCAA TATCCCCTCT AAAAAAAGCG GCGCGTGCTT TCAATGTGAG CCATAAGACT |
| 62401 | TCATCTGTCG GCTCTGGTGG TACTTGAGTC GAAAGCAGCG TTGATGGCGA TGTTGAAGCT |
| 62461 | GCAGCTTCGT ACTCCAAAAG CTGCTTTTTT GAAACCCCGA ATGCAGAACA CGTGAATAGT |
| 62521 | AAAGAAACTG CGAAAAAAAT GAATAGTTGC AAATATAAAA GCCCGTATTT TATCTCTCGC |
| 62581 | TTTAAATGTA ATCCTTTATT ACGAAATGTA CAATAATTAC GGGCTTTATA TCCATAAGAA |
| 62641 | AGCTCATTGG CTGTTATAGA TACTAAGGAT CTATTAAAGC CGGTTTAGAT TTGCTAGCAG |
| 62701 | ATGTTTTTAT CGTGTCTTGA GTCGATTTTA ACGTCTCCGT ACTCCTCTGC TGAGGAGGTT |
| 62761 | GAGGCGATGG TGCTTGACTG TATAACGAAG GTTGGCTCTC TTGTGTAATT GTAGACATGA |
| 62821 | GCTGCTGATT TAATCGTTGG CTTTGTGAGA TAGTGTCTTT GATATCTTTA TTTTGCTGCT |
| 62881 | GTAATTGCTC AGCGAGATCA GCTCGAGTTA GCCCACTTTT TACTTTATCT TTGGTTTTCT |
| 62941 | GATCTGTGCT GCCCATCAGC TGGTTAACTA CTTCAACTAA TCGACTCAAC ACGCTGTATA |
| 63001 | TCACATCTGC AGAAAAACCG CCGAGTAATG CAGCAAGTGG CTTATGAAAG TCACCCAAAG |
| 63061 | AAGAGTGATT TTGAGTTATC TCTTGGGTTG GGATCAGCTC CGCAATCATC AATCCCGCCA |
| 63121 | TAAAGCCCAT AATCACCATA GACCAATAAG TAGAATCAAA CTTGGGATCG TAATTGCATA |
| 63181 | ATCGAATAAA ATTTCGAGAT TTATGCAAAC AAGCAAATGT TGCTCCTAGC CCCGCGCAGC |
| 63241 | AGAGTAAAAA TAACTGATTC AGTAAGAGCA CCCTGCCTTC TGAGTTAAAT AAACCTTCAT |
| 63301 | TAATTGTTTT CTGATTGACT TCAGGTGATA TAGACAGAGA AATAATAGAA ATTAAAAAGA |
| 63361 | AAAGCGCCAA CAATGACATA TGTCGAACTA ACTTAACAGG CCCTAAAAAC CTTAACCAAC |
| 63421 | TCACCGAAGT GGACTCCTTA TCCATAATAG CAAGCGTCAA TGGCTTAGCT GGATAAACCA |
| 63481 | GTAAAGATAA CTCTTTATGG CACTTCGTCA GGGCAGTTAA CTCGTGCTCT AACATTAAGC |
| 63541 | TATAGCTATC TCCCTCTCTC ACCTCTTGCT TATTTTGTAA TTGATAAACT GTGTTCGGGA |
| 63601 | TATAGCTTGG CATGGTCTTT CCTTCGCCTG AAGCGTAGCG GATCATGACA TCGCATTCTG |
| 63661 | TCAGTAAATG ACTAACAAAG TCCTTCTTAG ACATAGTGTT ACCTATATTA AATGGCATCG |
| 63721 | AGTTGACGTT TATAACAGGG AATATAGTGT CACTTCAAGC ACATTTAATT ACAACTAACT |
| 63781 | GTAAAAGATA AATTAGGTCT TAGTTTAAAA TAAAACTAGT AATAAGAGAT GAAAGACACC |
| 63841 | AAGTAACGGC CGCGCAGATA AGCTTAAGCT TTTGAATAAT CCATTAAAGG TTATTAGCGT |
| 63901 | AATTAAGTGA TAGTGCGCTC CTCGTTTTGT GTAACGAGAT CCACCTCTAG ATATTAGGTG |
| 63961 | GGATCAATGT CAATTGGGTT TAATCGCAAC TTATGACGAG TCGTAGATTG GCTCGCCTTT |
| 64021 | TAACGGTTCT TCTAAGACTC ATTTAATTGC TAAATAATCA TCGTGCAGAG TTTGCTTAGC |
| 64081 | TAGTGATTAT ATTACGCTCT CCAAATTCAG TTTGAAAAGT ATGAACGCTA TTTAGGACTG |
| 64141 | AACAATAAAA ACAAAAAAGG CTCCCTAAGG AGCCTTTTTA CCTGAATGGT AAATGATTAC |
| 64201 | CAACCAGCTT TTTCTTTAAG TGCAGAACCG ATCTCAGCTA GAGAACGAAC AGTCTTAACG |
| 64261 | CCTGCTTCTT CTAGAGCTGC GAACTTCTCA TCAGCAGTAC CTTTACCGCC TGAGATGATT |
| 64321 | GCACCAGCGT GACCCATACG CTTGCCCGGA GGAGCAGTAA CACCAGCAAT GTAAGAAACC |
| 64381 | ACTGGCTTAG TCACGTTGTG TTTGATGTAC TCTGCAGCTT CTTCTTCTGC TGTACCACCG |
| 64441 | ATCTCACCAA TCATTACGAT TGCTTCAGTC TTAGGATCGT TCTGGAACAT TTCTAGTACG |
| 64501 | TCGATGAAGT TAGTACCTGG GATTGGGTCA CCACCGATAC CAACACAAGT TGATTGACCG |
| 64561 | AAACCAGCGT CAGTAGTTTG CTTAACTGCT TCGTAAGTAA GAGTACCTGA ACGAGATACG |
| 64621 | ATACCTACTT TACCAGGTAA GTGGATGTGA CCAGGCATGA TACCAATCTT ACACTCACCC |
| 64681 | GGAGTGATAA CACCTGGGCA GTTAGGACCG ATCATACGAA CGCCCGTTTC TTCTAGCTTC |
| 64741 | ACTTTAACAT CAACCATATC TAGTGTAGGG ATGCCTTCAG TGATACAAAC GATTAGCTTA |
| 64801 | ATGCCACCGT CGATAGCTTC TAAGATAGCG TCTTTACAGA ATGCAGCTGG TACGTAGATT |
| 64861 | ACTGTCGCAG TTGCGCCAGT TGCTTCTACA GCTTCACGTA CAGTGTTGAA TACTGGAAGA |
| 64921 | CCAAGGTGAG TTTGACCACC TTTACCAGGT GAAACACCAC CAACCATTTG CGTACCGTAC |
| 64981 | TGGATAGCTT GTTCTGAGTG GAAAGTACCC TGACCACCAG TGAAACCCTG ACAGATTACT |
| 65041 | TTAGTATCTT TATTAATTAG TACAGACATT ATTTGCCCTC CGCAGCAGCA ACTACTTTCT |
| 65101 | CTGCAGCATC AGTTAGTGAT TCAGCAGCGA TGATGTCAAG ACCAGAGTTA GCTAGTACTT |
| 65161 | CACGGCCAGC TTCAGCGTTA GTACCTTCAA GACGTACAAC TACAGGTACG CTTACACCAA |
| 65221 | CTTCTTTAAC TGCACCAATG ATACCTTCAG CGATCATGTC ACAACGAACG ATACCACCGA |
| 65281 | AGATGTTAAC TAGCACTGCT TTCACGTTGT CATCTGAAAG GATGATCTTG AATGCTTCAG |
| 65341 | ATACACGCTC TTTAGTCGCG CCGCCACCAA CGTCTAGGAA GTTAGCTGGC TTACCGCCGT |
| 65401 | GTAGGTTTAC GATGTCCATT GTACCCATCG CTAGGCCTGC ACCGTTAACC ATACAACCAA |
| 65461 | CGTTACCGTC TAGAGCAACG TAGTTTAACT CGAAGCTTGC AGCGTGAGCT TCACGTGCAT |
| 65521 | CTTCTTGTGA AGGATCGTGG AATTCACGGA TCTTAGGCTG ACGGAATAGC GCGTTGCCAT |
| 65581 | CAACACCAAT CTTGCCGTCT AGGCAATGTA GGTTGTTTTC GTCAGTGATT ACTAGAGGGT |
| 65641 | TGATCTCTAG AAGTGCGAAA TCGTGATCGA TGAACATGTT CGCAAGACCT AGGAAGATCT |
| 65701 | TTGTGAACTG TTTGATCTGT GCTGGGTTAA GACCAAGCTT GAAGCCTAGC TCACGACCTT |
| 65761 | GGTATGCCTG AGGGCCTACT AGTGGATCGA TTTCAGCTTT GTGAATTAGT TCTGGCGTTT |
| 65821 | CTTCAGCAAC TTGCTCGATT TCAACACCAC CTTCAGTTGA AGCCATGAAC ACGATTTTAC |
| 65881 | GTGAAGCACG GTCAACAACA GCACCAAGGT ATAGCTCATT TGCAATATCA GTGCAGCTTT |
| 65941 | CAACTAGGAT CTTAGCAACA GGCTGACCTT TTTCGTCAGT CTGGTAAGTT ACTAGGTTTT |
| 66001 | TACCTAACCA GTTTTCAGCA AATGCGCGGA TCTCGTCTTT GCTATCAGCT AGCTTAACAC |
| 66061 | CGCCAGCTTT ACCACGGCCA CCCGCGTGTA CTTGACATTT AACGACCCAC TTGTCGCCAC |
| 66121 | CAATTTTACC AGCAGCTTCA ACTGCTTCCT GAGGAGTGTC ACAAGCGTAA CCCTCAGACA |
| 66181 | CAGGTAAACC ATATTCGGCA AAAAGTTGTT TTGCCTGATA CTCATGCAAA TTCATGATGC |
| 66241 | TTTATCCAAT TTTTTATACT AAAAATGCTG AATATTTATG CAAATAATCA GCTATCCCAA |
| 66301 | ATTGCGCACA TAGTATAGAT CCCACGGCGT TTTAATAAAA CCCCAGTTAG ACCAAAGGCG |
| 66361 | CAAAAAAAAA GCTGTATGTT GCTTAATTGC ACGCATACAG CTTAAGATTT AATCTTTTGA |
| 66421 | TTAAACGTCT AGAAGTAGAC GTGTTGGATC TTCTAATAAT TCTTTGATTG TTACTAGGAA |
| 66481 | ACCAACTGAT TCTTTACCAT CGATTTGACG GTGGTCATAA GAAAGCGCTA GGTACATCAT |
| 66541 | AGGTAGAATT TCTACCTTAC CGTTTACAGC CATTGGACGC TCTTGGATCT TGTGCATACC |
| 66601 | AAGAATTGAA GACTGAGGTA GGTTGATGAT AGGCGTAGAA AGTAGTGAAC CGAATACACC |
| 66661 | ACCGTTTGTG ATTGTGAAGT TACCACCAGT CATATCATCA ACTGTTAGTT TACCATCACG |
| 66721 | ACCTTTAAGC GCTAGCTCAC GGATACCTTT TTCGATTTCA GCAACAGATA GCTTGTCACA |
| 66781 | GTCACGAAGT ACTGGTGTTA CTAGACCACG AGGTGTAGAA ACAGCGATGC TGATGTCGAA |
| 66841 | GTAGTTGTGA TAAACGATAT CATCACCGTC GATTGATGCA TTTACTTCAG GGAAACGCTT |
| 66901 | AAGTGCTTCT GTTACTGCTT TCACGTAGAA AGACATGAAA CCAAGACGAA TACCGTGACG |
| 66961 | CTCTTCAAAT ACATCTTTGT ACTGCTTACG AAGGTCCATG ATTGGCTTCA TGTTTACTTC |
| 67021 | GTTGAACGTA GTAAGCATTG CTGTTGAATT CTTTGCTTCA AGAAGACGGT TAGCAATTGT |
| 67081 | CTTACGAAGA CGTGTCATTG GAACACGCTT CTGCGTACGA TCACCTACTG GTGCTGCTGG |
| 67141 | CGCTGCCGCA GCTGCTTTAG CTGGTGCTGA TGCTGGCTTA GCTGCTGGCG CTTTAAGGAA |
| 67201 | TGCGTCAACA TCTTCTTTGG TGATGCGACC ACCTTTACCA GAGCCTTTGA TCTGAGAAGC |
| 67261 | GTCTAGGCCT TTTTCTGCAA TCAAGCGACG AACTGAAGGC GTAAGCACAT CAGCGTTTTC |
| 67321 | GTCAGACGTT GCTGGCGCTG CGTCAGAAGT TGCTGACGCT GCAGCTGGTG CACCACCTGC |
| 67381 | AGAAAGCTTA CCAATAACCT GCTCACCAAG TACTGTATCG CCTTCAGCGT GCAAGTGTTC |
| 67441 | ACCCATTACA CCGTCTTCAG GTGCAACAAC TTCTAAAACA ACTTTGTCTG TTTCGATGTC |
| 67501 | AACTAGGTTT TGATCACGGC TTACTGCCTC ACCTGGTTGA ACGTGCCAAG TTGCGATTGT |
| 67561 | AGCGTCTGCT ACTGACTCTG GAAGTACTGG TACTTTAATG TCCACTTCTT TACCTTCTGC |
| 67621 | TGCTGGCGCC GCTGCCGCTG GCGCTGCTGG TTGTTCATTT GATTGAGTTG GTGCCGCACC |
| 67681 | CGCAGCACCG ATTTGTGCAA TTACTTGCTC ACCTAATACT GTCGCGCCTT CTTCTTCAGA |
| 67741 | GATAGCAACA ATTACACCAT CTTCAGGTGC GACAACTTCT AAAACGACTT TATCCGTTTC |
| 67801 | GATGTCAACT AGGTTTTGGT CACGGCTTAC CGTATCACCA ACGCTTACAT GCCATGTCGC |
| 67861 | AACGGTCGCG TCTGCAACTG ACTCAGGAAG AACAGGCACC TTAATTTCGG TTGTCATCTC |
| 67921 | TTATTCCTTA TTTTTTAATT GTTAATGCGT CAGCAATCAA CGCGTTTTGT TCTTTAGTGT |
| 67981 | GGGTAGACAT ATATCCACAT GCCGGTGCAG CTGATGCCTT ACGGCCAGCA TATGTTAGGT |
| 68041 | TTGCACCTGC TGGGATTGCT TCCCAGAAAT GGTGTTGAGA ACAGTACCAA GCACCTTGGT |
| 68101 | TCTGTGGCTC TTCCTGACAC CATACGAAGT CTTTAACATG CTGGTAACGA GCCATGATCT |
| 68161 | CATCCATCTC TTTATGAGGG AATGGATATA ACTGTTCAAC ACGTACAATA GCAATATTGT |
| 68221 | TTAGTTCAAG CTTACGACGC TCTTGAAGTA GTTCGTAGTA AACCTTACCA CTACAGAATA |
| 68281 | CAACACGCTC TACGTTTTCC GGCTTAATCT CATCGATTTC ATCGATCATA TTGTGGAAAA |
| 68341 | CACCATCAGA CAGTTCTTCA AGACTAGAGA CTGCTAATGG GTGACGAAGC AATGATTTTG |
| 68401 | GTGTCATTAC AATCAATGGA CGACGTAAAG GACGTACTGA TTGACGGCGT AACATTGCAT |
| 68461 | AAACCTGCGC TGGCGTAGTT GGTACACATA CTTGCATGTT GTGGTCAGCA CAAAGCTGAA |
| 68521 | GGTAACGCTC CATACGTGAA GAACTGTGCT CTGGACCTTG ACCCTCGTAA CCGTGTGGAA |
| 68581 | GCAATAGAGT CAAGCCACAC AAACGGCCCC ACTTCTGCTC ACCCGAACTT AAGAATTGGT |
| 68641 | CAAATACAAC CTGTGCACCG TTCGCGAAGT CACCAAATTG TGCTTCCCAA AGAACCAGTG |
| 68701 | AAGTAGGCTC TGCAGTTGCG TATCCATATT CGAATGCTAA AACAGCTTCT TCAGAAAGTA |
| 68761 | CTGAGTCATA AACCTCAAAA GTACCCTGCT CTGGCCTGAT GTTTTGCAAT GGTAAGTAAG |
| 68821 | TAGACGCATC GTTTTGGTTA TGAACAACAG CGTGGCGGTG GAAGAAAGTA CCACGGCCAG |
| 68881 | AGTCCTGACC AGTTAGACGA ATGTCAGTGC CTTGATCAAC CATAGTCGCG TATGCAAGTG |
| 68941 | TCTCAGCCAT ACCCCAATCG AGAGGCTTAT CACCTTTAGC CATCGCTTTA CGGTCATCGT |
| 69001 | ATATTTTCTT AACACGAGAC TGTGCTTTGT GATCTTCTGG GTAGCTAGCA ACCTTTTCAC |
| 69061 | CAAGCTCTTT AAGCTTATCA ACAGAAACTT GCGTCTCATA AGGCGTATCC CAGTCATGAC |
| 69121 | CAACGTATTT CGACCACTCT GATGAATGCT TCGTTTCTGG TTGGATTTCA GAAACCACAC |
| 69181 | ATACACCATT GTCTAAGCCG TTTCGATAGT CATCAGCAAG CTGTTTTGCT TCTTGTGCTG |
| 69241 | ACAATACCCC TTCAGCGACA AGCTTGTCAG AATATAACTG ACGCGGTACT GGGTGCTTTT |
| 69301 | TGATCTTTTG ATACATTAGA GGCTGAGTTG CATTTGGCTC ATCAGCTTCA TTGTGACCGT |
| 69361 | GGCGACGGTA ACACACTAAA TCAATAACCA CATCACGCTT AAACTTGTTA CGGAAATCAA |
| 69421 | GCGCAATTTG TGTTACGAAT GCAACTGCTT CAGGATCGTC AGAGTTTACG TGGAAGATTG |
| 69481 | GTGCCTGAAC CATCTTAGCT ATGTCAGTAC AGTATTCTGT TGAACGCGTA TCTTCTGGTT |
| 69541 | TAGATGTTGT AAAACCAACT TGGTTGTTTA CAACGATACG AACAGTACCA CCAACACCGT |
| 69601 | ATGCACGTGT CTTTGATAAG TTAAATGTCT CTTGTACAAC ACCTTGGCCT GCAATTGCCG |
| 69661 | AGTCACCATG GATGGTGATT GGTAGTGCTT TAGAGCCACT TGCGCAATCT AAACGGTCAA |
| 69721 | GACGTGCTCT TACCGAGCCC ATTACCACTG GGTTAACGAT TTCTAAGTGA GACGGGTTAA |
| 69781 | AGGCCAATGC CATGTGAACA TCGCCGCCTT GTGTCGCGAA ATCAGAAGAG AAGCCCATGT |
| 69841 | GATATTTAAC ATCACCAGAA CCTGCAGACT CGCCATATTT ACCAGCAAAT TCATCGAATA |
| 69901 | ACTCTTGAGG GTTCTTACCA AGGACGTTAA CTAATACGTT AAGACGACCA CGGTGAGCCA |
| 69961 | TACCGATAAC AACTTCTTCT TGGCCACTTT CACCAGCTCT GTGTACCAGC TCTTTCAGCA |
| 70021 | TTGGTACAAG TGCATCGCCA CCTTCTAGTG AGAAACGTTT TGCACCTGGG AACTTAGCAC |
| 70081 | CAAGATATTT TTCAAGACCA TCTGCAGCGA TTAAACCTTG CAATAAGCGT AGTTTGTTTT |
| 70141 | CTTTGCTAAA TTGAGGCTTA GAGAAACCGG CTTCTAAGCG TTGTTGTAAC CAGCGCTTTT |
| 70201 | CTTCGGTTGA TGTGATGTGC ATGTACTCTG CACCAACTGA ACCACAGTAG GTAGACTTTA |
| 70261 | ACGCAGCGTA CAAGTCTTTT AATTTCATTG TCTCTTTGCC ACAGGCAAAA GAGCCAACGT |
| 70321 | TGAATTCTTT ATCGTGATCC ACATCATCTA AATCGTGGTA AGCCAACTCG AGTTCTCTCA |
| 70381 | CTCGGTCACG TTGCCATAAA CCCAACGGGT CTAGATTGGC ATTTTGGTGG CCCCTAAATC |
| 70441 | TAAATGCATT AATAAGCTGC AACACACGCA CTTGTTTTGC ATCTGCCGCA CCTTCTGCAG |
| 70501 | AAACTACTAC TTCTCTGTGT TTGTTCTTAG CAAGCTCAGC AAATTGTGCT CTTACATCGG |
| 70561 | AATGTTTAAT ATCAACATCA ACACCTTCTA CTTTAGGCAG TTGATCAAAC ACTTCTCGCC |
| 70621 | ATTCTTCTGG CACTGAAGCC GCATCATCAA GATACGCCTC ATATAAATCT TCTACATATG |
| 70681 | CAACGTTACC GCCGTATAAG TGAGAAGATT CCAGCCATGC TTTCATCACA CCTTCGTGCA |
| 70741 | TTTATTAGCC CTTTTCTCTA GCGCAGAAAC TTAAACTTAA ACGAAAACAA GATGGCATGC |
| 70801 | TCAGCATGCC ATCTTAAGAT AATAATATGC TTTAAACCGA ACGGTTTAAT AGCATAGATT |
| 70861 | TAATGTGTCC AATCGCTTTG GTTGGATTCA AACCTTTCGG ACAAACGCTA ACACAGTTCA |
| 70921 | TGATACTGTG ACAACGGAAT ACGCTGAACG CGTCATCAAG ACCAGCTAAA CGCTCTTCAG |
| 70981 | TTGCTGTATC GCGGCTATCT GCCAAGAAGC GATACGCATG AAGAAGGCCC GCAGGACCGA |
| 71041 | TAAATTTATC TGGGTTCCAC CAGAATGATG GGCATGAAGT AGAACAACAT GCACATAAAA |
| 71101 | TACACTCATA AAGACCATCT AGCTTTTCAC GGTCTTCAAC AGATTGAAGA CGCTCTTCAC |
| 71161 | CGCCAGTTGG CTTATCATTG ATCAAGTAAG GTTTTACTTT CTCGTATTGA GTGTAGAACT |
| 71221 | GACTCATGTC AATTACCAAG TCACGAATTA CTGGCAAGCC AGGAAGTGGA CGTACGATAA |
| 71281 | TTTTACCTTT GCCATTCTTT TGTAGAGCAG ACAATGGAGT AATACATGCA AGGCCATTTT |
| 71341 | TACCATTCAT GTTTAGACCA TCAGAACCAC AAACACCTTC ACGACATGAA CGACGGAAAG |
| 71401 | AAAGTGTTGA GTCCTGCTCT TTTAACATAA GTAGTGCGTC AAGCACCATC ATGTCACGAC |
| 71461 | CTTCTTCAAC CTCAAGTTTG TATTCCTGCA TACGAGGTGC AGTATCAACA TCTGGGTTGT |
| 71521 | AACGATAAAC AGATAATTCT AAAGTTGCTG TTGCCATCGT CTAACTCCTA GTACGTACGA |
| 71581 | GCTTTAGGTG GGAATGCTTC ACGCGTAGTC GGCGCAAAGT TAACATCACG CTTTGTCATT |
| 71641 | GACTCACTTT CTGGGTGATA CATTGAGTGG CATAGCCAGT TCTCGTCATC ACGCTCTGGG |
| 71701 | AAGTCAAATC TTGAGTGCGC ACCACGGCTT TCCGTACGGA AGTTAGCAGC AACTGCTGTT |
| 71761 | GAGTATGCTG TTTCCATCAA GTTATCTAGT TCAAGACACT CAATACGTTG CGTGTTGAAT |
| 71821 | TCAGTTGACT TGTCATCAAG ACGCGCATAC TGAAGACGTT CACGGATTTC TTTAAGCTGC |
| 71881 | TTAAGGCCAT CAGCCATTGC ATCACCTTCA CGGAATACCG AGAAGTTAAG CTGCATACAC |
| 71941 | TCTTGCAGAT CTTTCTTGAT TTGAACTGGG TCTTCACCCT TACCAACTTC AGAAGTTTCC |
| 72001 | CAACGATTGT AACGAGCAAA CGCTGCATCA ACATCGTCTT TAGACGCTTC ACCAGTTGAC |
| 72061 | TCAAAGTCTT TCAAGTAAGA ACCTAGGAAG TTACCTGCTG CACGACCGAA TACAACTAAG |
| 72121 | TCAAGTAGCG AGTTACCACC TAGACGGTTT GCACCATGTA CTGATACACA AGCAATCTCA |
| 72181 | CCTACTGCGA ATAGACCTTC AACGATACGT TCATTACCGT TGTTGTCTAC GTCAAGAACT |
| 72241 | TGACCGTTTA CATTTGTAGG TACACCACCC ATCATGTAGT GACATGTTGG GATTACTGGA |
| 72301 | ATTGGCTCTT TAGCTGGGTC TACGTGTGCG AATGTTTTTG ATAGGTCACA AACGCCTGGT |
| 72361 | AGACGAAGGT TAAGCATCTC TTCACCTAAG TGGTCAAGTT TCAGCTTAAT GTGAATACCC |
| 72421 | CATGGGCCTT CACAACCACG ACCTTCGCGG ATCTCAGTCA TCATTGCACG TGCAACAACG |
| 72481 | TCACGACCAG CAAGGTCTTT AGCATTAGGG GCATAACGTT CCATGAAACG TTCGCCATCT |
| 72541 | TTATTCAGAA GGTAACCACC TTCACCACGA CAACCTTCTG TTACTAGCGT ACCAGCACCT |
| 72601 | GCGATGCCTG TCGGGTGGAA CTGCCACATC TCCATGTCTT GCATGCCGAT GCCAGCGCGT |
| 72661 | ACTGCCATAC CAACACCGTC ACCAGTGTTA ATGTGTGCAT TTGTTGTTGA TGCATAGATA |
| 72721 | CGACCAGCAC CACCAGTTGC AAGTACAACA GCTTTAGACT TGAAGTAAAC AACTTCACCA |
| 72781 | GTCTCGATCT CAATCGCTGT AACACCAACA ACGTCGCCTT TGTCATTTTT AACTAGGTCA |
| 72841 | AGGGCATACC ACTCAGAGAA AACGTTTGTT TTATTTTTAA CGTTTTGTTG GTATAGGCAA |
| 72901 | TGAAGAAGTG CGTGACCAGT ACGGTCAGCA GCTGCTGCTG TACGTGCAGC CTGCTCGCCG |
| 72961 | CCAAAATGCT TTGACTGACC ACCAAAAGGA CGCTGATAAA CACGGCCATT TTCAAAACGA |
| 73021 | GAAAATGGTA AGCCCATGTT TTCTAATTCA GTAATAGCTT CAGGACCCGT TTTAGTCATA |
| 73081 | TATTCGATAG CGTCTTGGTC ACCGATATAA TCAGAACCTT TAACAGTATC GTACATGTGC |
| 73141 | CATTCCCAGT TATCTTCATG AGAGTTGCCA AGTGCTACCG TAATACCACC TTGCGCAGAT |
| 73201 | ACTGTGTGAG AACGAGTTGG GAATACTTTA GAGATAAGAG CACATGTCTT GCCTGACTCA |
| 73261 | GAAATAGCAA GTGCAGCACG CATACCCGCA CCGCCGGCTC CAACAACTAC AGCATCAAAT |
| 73321 | TCACGGACAT TATATTTCAC TTAAACACCC CACAGAACAA ACAAACCAAC AGCAACATAT |
| 73381 | GCTAGTGCCA TTAGATTTAA TACAAAGCCT AAAACTGAAC GCATTGTTGA ACACTTGACG |
| 73441 | TAGTCAGTAA GAACTTGCCA AAGACCAATA CGAGTATGAA CCATTATACA AACTAGTGTA |
| 73501 | ATAATAGTTG CAGCTTTCAT AGCTAAGTTG TCGAACAACC CAGTCCATGC TTCATACGTT |
| 73561 | AATTCAGGAG TTAACGCTAA GTAGCCCACG ATAAATACAG CGTATGCTGC AATAATTAAT |
| 73621 | GCTGTTGCGC GAAGTGATAC ATAATCTTGT ACACCATCAC GTTTCAGAGT TGCTTGATTT |
| 73681 | AAGACCATAT CCACACTCCA CCAAGAATCG CAACGATTAC CCAAATGGCG ATTGCAATTT |
| 73741 | TAGCACTTGC ATTGCCCGAC TCTAACTCTT CCCAGTGGCC CATGTCTTGA ATCATGTGAC |
| 73801 | GGATACCGCC AATGATGTGG TAAGACAACA CAGCCAAGGT GCCCCACGCA ATGAATTTTG |
| 73861 | CAACAAAGCC AGTCATAAGC TCTTTTACAA ATTCAAAACC TTCAGGAGAA GAGAGAGATT |
| 73921 | CAGACCACGC CCAAATGACA AATGTCAGCG CGAAGAACAA CGCGACACCA GTGACACGAT |
| 73981 | GTAAGATCGA CGCCTTTGCC GTTGCTGGCA TAGATATAGT CGTAAGATCT AGATTTACAG |
| 74041 | GTCTTTGCTT TTTCACAGTT ACTTGCCCAT CTTTGCTCGA TAAGAGCTTC AT |
| // | |

### Bacterial Growth Conditions

Any set of known growth conditions can be used to practice embodiments as provided herein, for example, as described in US 2016-0237398 A1, or WO/2015/058179; exemplary growth conditions and parameters are described in Example 1, below.

### Making and Assembling Products of Manufacture

In alternative embodiments, products of manufacture as provided herein are comprised of recombinantly generated or substantially isolated components: (a) a recombinant bacterial Contractile Injection System (CIS) or a Metamorphosis Associated Contractile structure (MAC) formed or configured to comprise a tube having an inner core, (b) a Metamorphosis-Inducing Factor 1 (Mif1) protein positioned in the inner core of the tube of the CIS or MAC, (c) a chaperone 605 protein non-covalently associated with the Mif1 protein positioned in the inner core of the tube of the CIS or MAC, and (d) a proteinaceous cargo, or a heterologous protein or peptide, or compound, non-covalently associated or covalently associated or linked to the Mif1.

In alternative embodiments, CIS and MACs, including Mif1 and chaperone 605 protein, and payloads, as used in products of manufacture as provided herein are produced (synthesized) and fully assembled in vivo by bacteria such as P. luteoviolacea. In alternative embodiments, the bacteria also produce, synthesize or manufacture the payload to be delivered, and the CIS or MAC is assembled in vivo with (or including) the payload loaded or assembled in the inner core or tube of the MAC or CIS. In alternative embodiments, products of manufacture as provided herein, including CIS and MACs, Mif1, chaperone 605 protein, and payloads, are produced (synthesized) and fully assembled as described in the art for example, in Ericson et al, “A contractile injection system stimulates tubeworm metamorphosis by translocating a proteinaceous effector.” Elife 8 (2019): e46845.

Translocation mechanisms of effectors via the spike complex of a CIS have been well characterized; for example, in alternative embodiments, CIS and MACs, Mif1, chaperone 605 protein and payloads as used in products of manufacture as provided herein are produced (synthesized) and fully assembled using protocols and components as described for example, by Quentin et al., 2018, Nat Microbiol 3:1142-1152; and/or Shneider et al, 2013, PAAR-repeat proteins sharpen and diversify the Type VI secretion system spike. Nature 500:350-353; additional guidance for alternative pathways for loading effectors into the inner tube lumen can be found for example in Heymann J B, et al, 2013, Three-dimensional structure of the toxin-delivery particle antifeeding prophage of Serratia entomophila. J Biol Chem 288:25276-25284; and/or Sana T G, et al, 2016, Salmonella typhimurium utilizes a T6SS-mediated antibacterial weapon to establish in the host gut. Proc Natl Acad Sci 113:E5044-E5051, and/or Silverman J M, et al, 2013, Haemolysin Co-regulated Protein is an Exported Receptor and Chaperone of Type VI Secretion Substrates. Mol Cell 51, describing how effectors were found to interact with the inner tube protein (hcp) and are released post-firing by tube dissociation in the target cytoplasm.

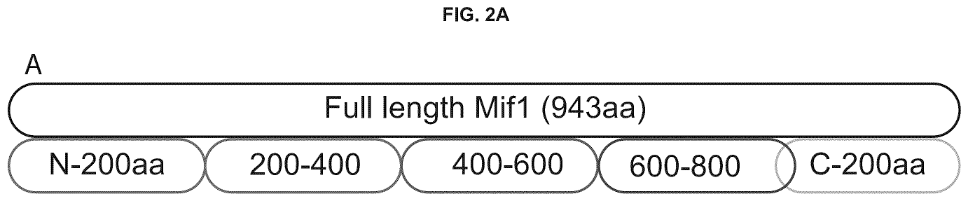

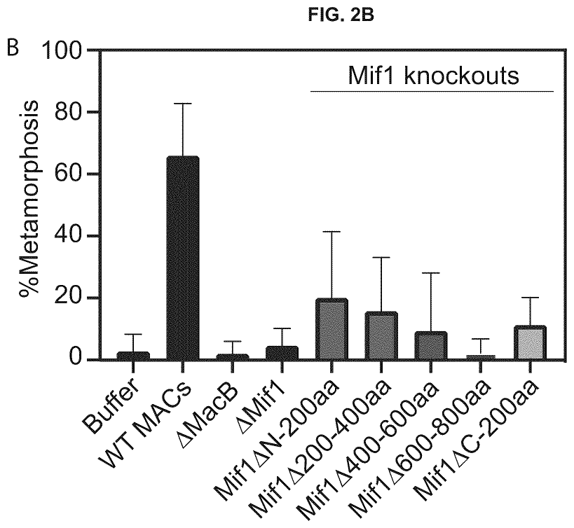

Our results directly showed the previously hypothesized possibility of effector delivery via the tube lumen of a CIS (Heymann et al., 2013; Sana et al., 2016; Shneider et al., 2013; Silverman et al., 2013). Interestingly, the comparison of MACs with a different class of CIS, namely the Type Six Secretion System (T6SS), reveals significant differences. The T6SS effectors that are thought to be delivered by the T6SS tube lumen show protein-protein interactions between the T6SS effector and the T6SS tube protein (Hcp) (Sana et al., 2016; Silverman et al., 2013). By contrast, we did not detect such interactions between Mif1 and MAC tube protein. One possible explanation could be that the biophysical characteristics of the T6SS tube and the MAC tube are different. While the T6SS tube is inherently unstable and disassembles soon after contraction, see for example, Szwedziak P, et al, 2019, Bidirectional contraction of a type six secretion system. Nat Commun 10:1565, inner tubes of MACs and other extracellular CISs (and contractile phages) can be readily detected by electron microscopy and therefore seem to be much more stable. Given our observation that expelled MAC tubes were always empty, this poses the question of how the effectors exit such a stable tube after contraction. We hypothesize that this could be the very reason for weak or entirely absent interactions between Mif1 and MAC tube, as well as for the low-density region that was seen in subtomogram averages separating Mif1 and MAC tube (FIG. 2B). Another mechanistic consequence of low affinity between Mif1 and tube could be the requirement of an assembly factor, i.e. JF50_12605, that allows for efficient targeting of Mif1 to the tube.

In one alternative embodiment, an exemplary CIS or MAC purification scheme comprises:

P. luteoviolacea was grown in 50 ml SWT media in 250 ml flasks at 30° C. for 6 hours or overnight (12-14 h). Cells were centrifuged for 30 minutes at 4000 g and 4° C. and resuspended in 5 ml cold extraction buffer (20 mM Tris, pH 7.5, 1M NaCl). Cultures were centrifuged for 30 minutes at 4000 g and 4° C. and the supernatant was isolated and centrifuged for 30 minutes at 7000 g and 4° C. The pellet comprising the isolated CIS or MAC was resuspended in 20-100 μl cold extraction buffer and stored at 4° C. for further use.

In alternative embodiments, all, several of any one of the components of a product of manufacture as provided herein is heterologous to the assembling bacteria, where optionally the bacteria gains the ability to produce or internally synthesize the component by insertion of one or more recombinant nucleic acid(s) that encode for that component or components.

### Formulations

In alternative embodiments, provided are formulations, including pharmaceutical formulations, comprising products of manufacture as provided herein for delivering a proteinaceous cargo, a protein or peptide, a drug or a marker, to or into a cell such as a eukaryotic cell, wherein optionally the delivery of the formulation or composition with the eukaryotic cell is in vitro, ex vivo, or in vivo. For example, in alternative embodiments, substantially purified or isolated bacterial CIS or MACs, or the recombinant bacterial CIS or MACs, or liposomes or lipid-comprising nanoparticles incorporating or expressing on their outer surface the substantially purified or isolated bacterial CIS or MACs, or the recombinant bacterial CIS or MACs, as provided herein, are formulated in sterile saline or buffered formulations. In alternative embodiments, formulations as provided herein comprise water, saline, a pharmaceutically acceptable preservative, a carrier, a buffer, a diluent, an adjuvant or a combination thereof.

In alternative embodiments formulations as provided herein are administered orally or rectally, or are formulated as a liquid, a food, a gel, a candy, an ice, a lozenge, a tablet, pill or capsule, or a suppository or as an enema formulation, or for any form of intra-rectal or intra-colonic administration.

In alternative embodiments, formulations are provided herein are administered or are delivered in vivo by any effective means appropriated for a particular treatment. For example, depending on the specific agent to be administered with (by carried by) a CIS or MAC-comprising formulations as provided herein, a suitable means can include oral, rectal, vaginal, nasal, pulmonary administration, or parenteral (including subcutaneous, intramuscular, intravenous and intradermal) infusion into the bloodstream. For parenteral administration, CIS or MAC-comprising formulations as provided herein can be formulated in a variety of ways. Aqueous solutions of the modulators can be encapsulated in polymeric beads, liposomes, nanoparticles or other injectable depot formulations known to those of skill in the art. In alternative embodiments, CIS or MAC-comprising formulations as provided herein are administered encapsulated in liposomes (see below). In alternative embodiments, depending upon solubility, compositions are present both in an aqueous layer and in a lipidic layer, for example, a liposomic suspension. In alternative embodiments, a hydrophobic layer comprises phospholipids such as lecithin and sphingomyelin, steroids such as cholesterol, more or less ionic surfactants such a diacetylphosphate, stearylamine, or phosphatidic acid, and/or other materials of a hydrophobic nature.

In alternative embodiments, formulations are provided herein are formulated in any way and can be administered in a variety of unit dosage forms depending upon a desired result, for example, a condition or disease and the degree of illness, the general medical condition of each patient, the resulting preferred method of administration and the like. Details on techniques for formulation and administration are well described in the scientific and patent literature, see, for example, the latest edition of Remington's Pharmaceutical Sciences, Maack Publishing Co., Easton PA (“Remington's”).

For example, in alternative embodiments, CIS or MAC-comprising formulations as provided herein are formulated in a buffer, in a saline solution, in a powder, an emulsion, in a vesicle, in a liposome, in a nanoparticle, in a nanolipoparticle and the like. In alternative embodiments, the compositions can be formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature. Formulations and/or carriers used to practice embodiments as provided herein can be in forms such as tablets, pills, powders, capsules, liquids, gels, syrups, slurries, suspensions, etc., suitable for in vivo, in vitro or ex vivo applications.

In practicing embodiments as provided herein, product of manufacture, or CIS or MAC-comprising, formulations as provided herein can comprise a solution of compositions disposed in or dissolved in a pharmaceutically acceptable carrier, for example, acceptable vehicles and solvents that can be employed include water and Ringer's solution, an isotonic sodium chloride. In addition, sterile fixed oils can be employed as a solvent or suspending medium. For this purpose any fixed oil can be employed including synthetic mono- or diglycerides, or fatty acids such as oleic acid. In one embodiment, solutions and formulations used to practice embodiments as provided herein are sterile and can be manufactured to be generally free of undesirable matter. In one embodiment, these solutions and formulations are sterilized by conventional, well known sterilization techniques.

Product of manufacture, or CIS or MAC-comprising formulations, as provided herein can comprise auxiliary substances as required to approximate physiological conditions such as pH adjusting and buffering agents, toxicity adjusting agents, for example, sodium acetate, sodium chloride, potassium chloride, calcium chloride, sodium lactate and the like. The concentration of active agent in these formulations can vary widely, and can be selected primarily based on fluid volumes, viscosities and the like, in accordance with the particular mode of in vivo, in vitro or ex vivo administration selected and the desired results.

Product of manufacture, or CIS or MAC-comprising formulations, as provided herein can be delivered by the use of liposomes. In alternative embodiments, by using liposomes, particularly where the liposome surface carries ligands specific for target cells or organs, or are otherwise preferentially directed to a specific tissue or organ type, one can focus the delivery of the CIS or MAC-comprising formulations, and thus an active agent, to or into a target cells in an in vivo, in vitro or ex vivo application.

Product of manufacture, or CIS or MAC-comprising formulations, can be directly administered, for example, under sterile conditions, to an individual (for example, a patient) to be treated. The modulators can be administered alone or as the active ingredient of a pharmaceutical composition. Compositions and formulations as provided herein can be combined with or used in association with other therapeutic agents. For example, an individual may be treated concurrently with conventional therapeutic agents.

### Nanoparticles, Nanolipoparticles and Liposomes

Provided are nanoparticles, nanolipoparticles, vesicles and liposomal membranes comprising product of manufacture, or CIS or MAC-comprising formulations, as provided herein. Provided are multilayered liposomes comprising compounds used to practice embodiments as provided herein, for example, as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice embodiments as provided herein.

Liposomes can be made using any method, for example, as described in Park, et al., U.S. Pat. Pub. No. 20070042031, including the method of producing a liposome by encapsulating an active agent (for example, CIS or MAC-comprising formulations as provided herein), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice embodiments as provided herein comprise a substituted ammonium and/or polyanions, for example, for targeting delivery of a compound as provided herein, or a compound used to practice methods as provided herein, to a desired cell type or organ, for example, brain, as described for example, in U.S. Pat. Pub. No. 20070110798.

Provided are nanoparticles comprising compounds as provided herein, for example, used to practice methods as provided herein in the form of active agent-containing nanoparticles (for example, a secondary nanoparticle), as described, for example, in U.S. Pat. Pub. No. 20070077286. In one embodiment, provided are nanoparticles comprising a fat-soluble active agent used to practice embodiments as provided herein, or a fat-solubilized water-soluble active agent to act with a bivalent or trivalent metal salt.

In one embodiment, solid lipid suspensions can be used to formulate and to deliver compositions used to practice embodiments as provided herein to mammalian cells in vivo, in vitro or ex vivo, as described, for example, in U.S. Pat. Pub. No. 20050136121.

### Delivery Vehicles

In alternative embodiments, any delivery vehicle can be used to practice the methods as provided herein, for example, to deliver products of manufacture, or CIS or MAC-comprising formulations, as provided herein, to mammalian cells, for example, in vivo, in vitro or ex vivo. For example, delivery vehicles comprising polycations, cationic polymers and/or cationic peptides, such as polyethyleneimine derivatives, can be used for example as described, for example, in U.S. Pat. Pub. No. 20060083737.

In one embodiment, a dried polypeptide-surfactant complex is used to formulate CIS or MAC-comprising formulations as provided herein, for example as described, for example, in U.S. Pat. Pub. No. 20040151766.

In one embodiment, compounds and compositions as provided herein, or a compound used to practice methods as provided herein, can be applied to cells using vehicles with cell membrane-permeant peptide conjugates, for example, as described in U.S. Pat. Nos. 7,306,783; 6,589,503. In one aspect, the composition to be delivered is conjugated to a cell membrane-permeant peptide. In one embodiment, the composition to be delivered and/or the delivery vehicle are conjugated to a transport-mediating peptide, for example, as described in U.S. Pat. No. 5,846,743, describing transport-mediating peptides that are highly basic and bind to poly-phosphoinositides.

In one embodiment, electro-permeabilization is used as a primary or adjunctive means to deliver the composition to a cell, for example, using any electroporation system as described for example in U.S. Pat. Nos. 7,109,034; 6,261,815; 5,874,268.

### Dosaging

In alternative embodiments, product of manufacture, or CIS or MAC-comprising formulations, as provided herein, including pharmaceutical compositions, are administered for prophylactic and/or therapeutic treatments. In therapeutic applications, compositions are administered to a subject, for example, a human in need thereof, in an amount of the agent sufficient to cure, alleviate or partially arrest the clinical manifestations and/or its complications (a “therapeutically effective amount”).

The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. Dosage levels may range from about 0.01 mg per kilogram to about 100 mg per kilogram of body weight. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, for example, Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods as provided herein are correct and appropriate.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition. For example, in alternative embodiments, a substantially purified or isolated bacterial CIS or MACs is at least about 90%, 95%, 97%, 98%, 99% or 99.5%, or more pure, or is between about 85% and 99.5% pure, or having no more than about 10%, 9%, 8%, 7%, 6%, 5%, 4%, 3%, 2%, 1%, 0.5%, 0.1%, 0.05%, or 0.01% non-bacterial CIS or MAC elements.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the following examples; however, it is to be understood that the invention is not limited to such examples.

### EXAMPLES

### Example 1: Bacteria Stimulate Tubeworm Development by Injecting a Protein Toxin

This example describes exemplary compositions and methods for practicing embodiments as provided herein.

Contractile Injection Systems (CIS) are nanometer-scale syringe-like machines that bear homology to the contractile tails of bacteriophage. The CIS structure contains a contractile sheath and rigid inner tube, that upon contraction can penetrate membranes and injecting a protein cargo. Pseudoalteromonas luteoviolacea is a marine bacterium that produces metamorphosis associated contractile injection systems (MACs) a type of extracellular CIS which is required for the stimulation of metamorphosis of a marine invertebrate Hydroides elegans. This contractile injection system is made up of an array of tails held together in a hexagonal lattice and contain approximately 100 contractile tails. This protein complex contains an effector protein in its inner tube and upon contraction injects this protein into the host cells. This protein is both necessary and sufficient to induce the tubeworm metamorphosis. How this protein functions and the requirements for loading into the complex were previously unknown.

Here we show that Mif1 requires interaction with the chaperone 605 protein for Mif1 to be loaded within the complex and that this loading is required for metamorphosis. Furthermore, we show Mif1 is a toxin, and this toxin activity is found in the C-terminal portion of the protein. We identified Mif1 is a membrane associated protein and possesses lipase activity. Through fragmentation analysis we identify a portion of the Mif1 protein associates with membrane lipid and this region is required for lipase activity. We show that the protein N and C terminus work cooperatively to maintain this function. Our data shows the loading requirements for the effector Mif1 protein and its function as a lipase toxin. These data suggest a role for lipid cleavage in the initiation of tube worm metamorphosis and provides context for the findings previously published showing how lipids and PKC signaling can stimulate tubeworm metamorphosis. Understanding the role that bacteria contribute to animal development and the mechanisms by which they are capable of interacting furthers our understanding of animal-bacteria interactions and their role in animal development.

We have shown that the Mif1 protein effector loaded within the inner tube lumen of the MACs structure stimulates the metamorphosis of Hydroides larvae.

Here we show the regions required for Mif1 loading into the MACs complex and its effect on Hydroides metamorphosis. To determine how these regions might be used for loading of the protein, we further looked into their ability to associate with the 605-chaperone protein which is also required for Mif1 loading into the complex. We then look at the protein folding prediction using ALPHAFOLD2™ to predict a possible function for Mif1 once injected into the host cells. Our findings show that the Mif1 protein appears to be a porin? and we further validate this finding by determining lipid binding and association with the membrane. Furthermore, we identify that Mif1 purification is associated with lipase activity and the C terminal region retains some of this activity. The A portion of the protein appears to possess a lipase chaperone motif and may contribute to the C-terminal function. Our results show that the N and C term domains are required for 605 chaperone binding, subsequent protein complex loading and metamorphosis.

### Results and Discussion

### Structural Prediction of Mif1 Identifies Three Domains and Suggests Membrane Association

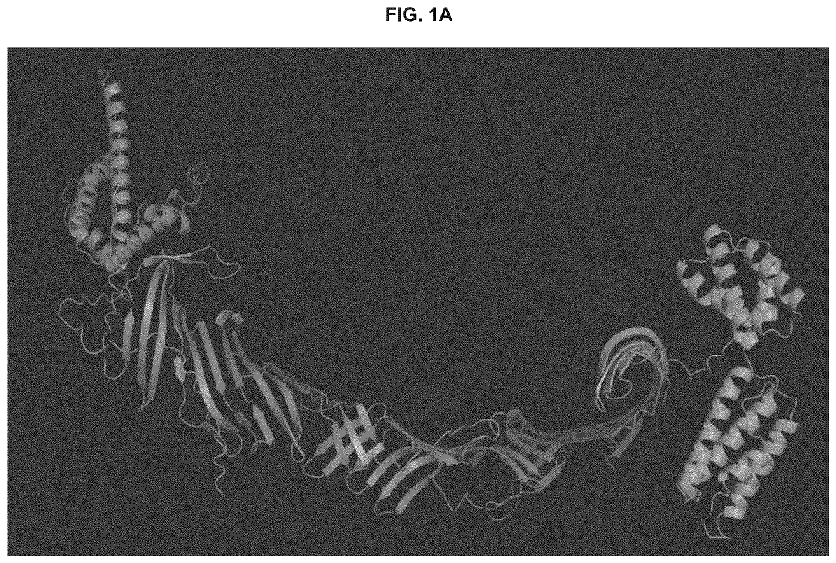

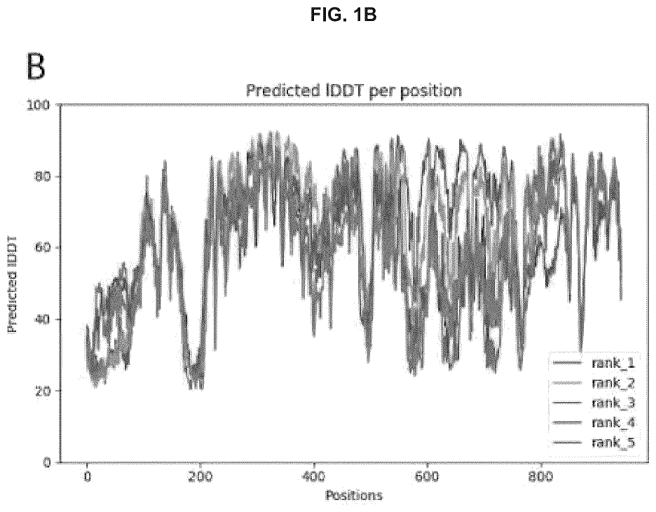

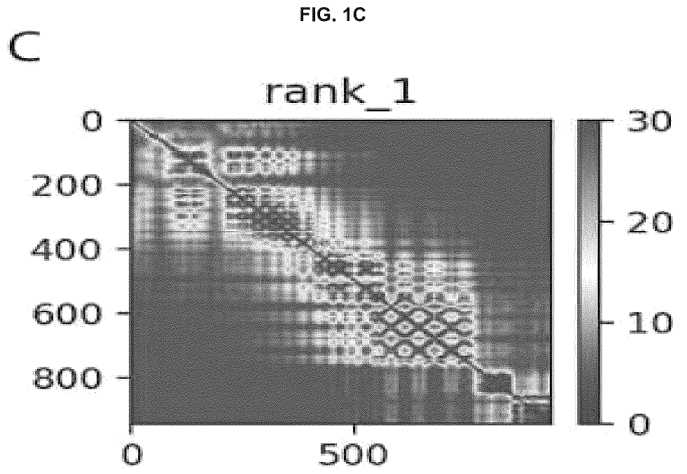

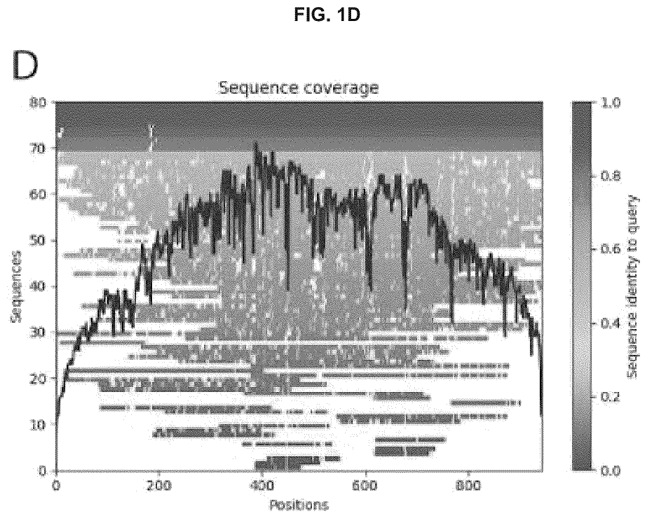

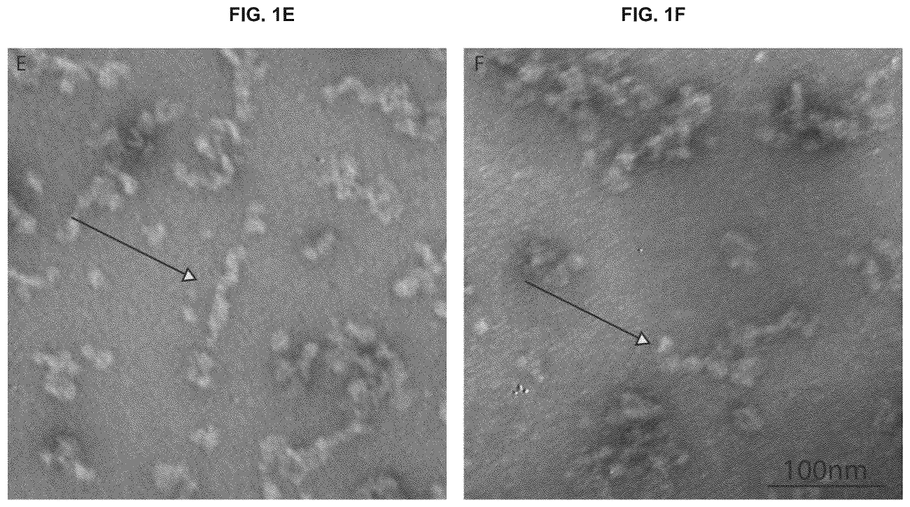

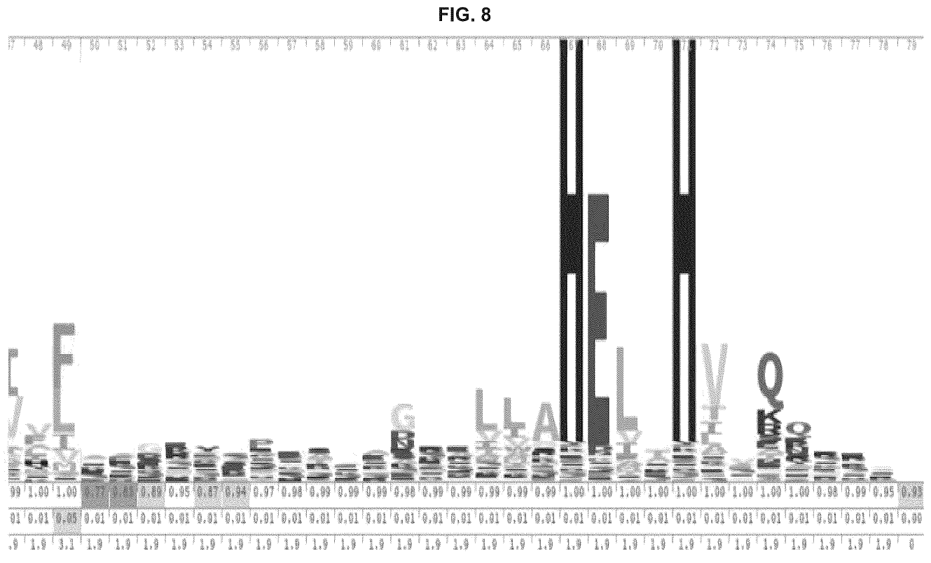

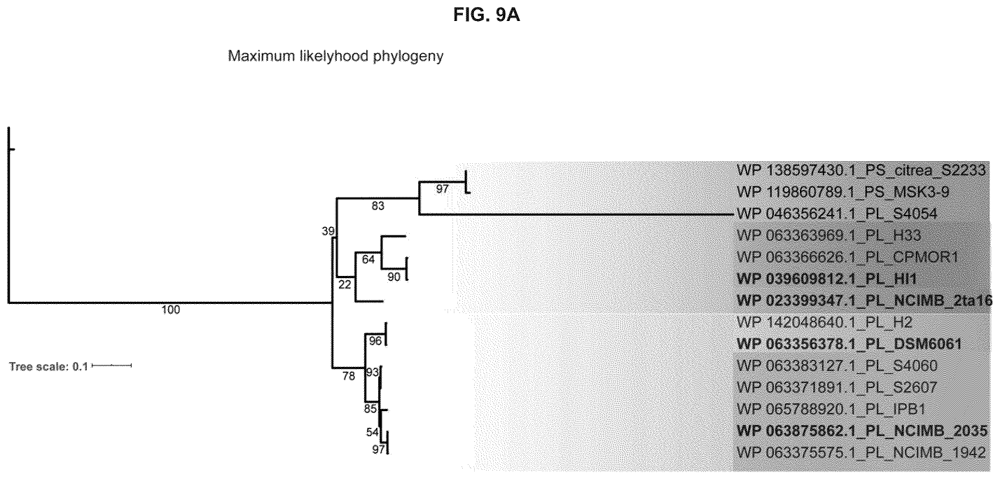

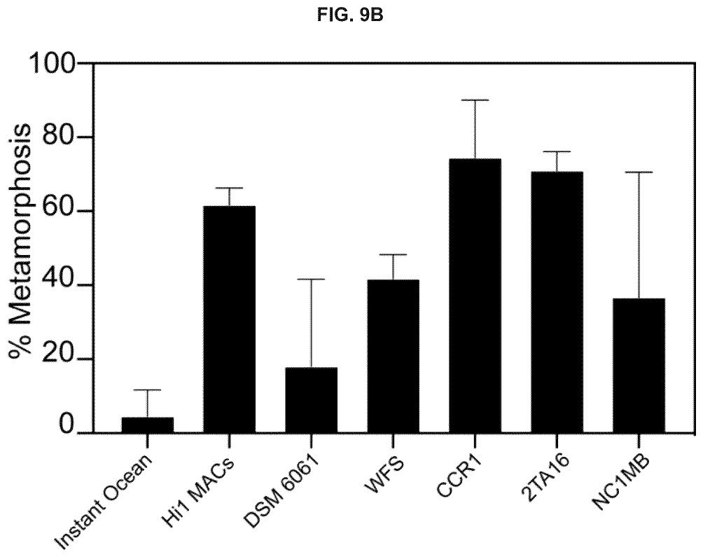

To determine the structure of Mif1 the protein sequence was analyzed for homology and predicted domains via BLAST™, HMMR™, and PHYRE2™ (Creative Commons Attribution-2.0); however, no confident predictions were given (FIG. 8). The sequence appeared to share little homology with known protein structures, only sharing some homology to domain of unknown function 4157, a suspected metallopeptidase but lacking the conserved motif of (HExxH) (see FIG. 9B). To circumvent this issue the program ALPHAFOLD2™ (DeepMind, London, UK), a protein 3d structure prediction software was used to predict the entire protein structure. The structure showed two alpha helical globular domains at the N and C-terminus of the proteins, with a large beta pleated sheet portion in the middle of the protein (FIG. 1, A). The structure resembled many known membrane transporter proteins which have a beta-pleated sheets that circularize and allow hydrophilic components to pass through the membrane⁴⁰.

This observation was searched against the protein data base (PDB) or structural classification of proteins (SCOP) database using 3D-Blast, a search engine using known crystal structures to identify homologs, we found the top predicted structures which hit our predicted structure listed in (Table 1 and table S1). The hits matched to the center beta-pleated sheets portion of the protein and the hits all resembled some form of membrane associated protein complexes such as transporter proteins, membrane porins, and other membrane bound complexes.

FIG. 1. Mif1 alpha fold prediction. (A) ALPHAFOLD2™ prediction of the effector protein Mif1. (B) Predicted IDDT local superposition-free score for each residue 1-943. (B) predicted alignment error of predicted residues vs scored residues. (C) Sequence coverage of predicted residues. (E,F) Negative staining Transmission electron microscopy of purified Mif1. Scalebar=100 nm.

Membrane depolarization has been a proposed method of metamorphosis induction due to high concentrations of K⁺ inducing metamorphosis⁴¹. It is also possible for the protein to have multiple functions as the ALPHAFOLD2™ model appears to predict 3 separate domains as seen by the separate nodes seen in the sequence coverage of predicted residues, which can be useful in identifying domains (FIG. 1, C). To determine if these domains may contain independent activity, we ran these domains independently through ALPHAFOLD™ to determine if 3D-Blast would predict structures other than the membrane associated complexes found during the entire protein search. The First alpha helical domain (amino acids 1-200) search revealed a homology to a known protein lipase chaperone structure (sup figure x, A) (pdb 2ES4). The central region containing the beta-pleated sheets was searched independently (amino acids 200-760) and results mirrored those of the full-length protein. The domain which exists in the C-terminus (amino acid 760-943) appeared to hit a few different structures but interestingly the C-term appeared to hit against a 14-3-3 zeta (pdb 1IB1). These Findings together suggest that Mif1 is a membrane associated protein and likely has distinct functions associated with three different domains found on the protein. The N and C terminal domain may contain protein binding domains or enzymatic activity and the Central beta-pleated sheet domain may anchor the protein in the membrane and potentially facilitate transport of molecules.

TABLE 1
3D-Blast hits using ALPHAFOLD2 ™ predicted model of Mif1 (1-943aa)
Search Results of 3D-BLAST
Query Protein: Mif1, Search Database: PDB (29-May-10)

| # | Protein | Length | Score | E-value | % Iden | % Gaps | Classification |
|---|---|---|---|---|---|---|---|
| 1 | 2vqi: | 542 | 199 | 2.00E−50 | 31.5 | 20.7 | Transporter |
| 2 | 1nqf: | 554 | 196 | 1.00E−49 | 30.7 | 25.5 | Transport |
| | | | | | | | protein |
| 3 | 3m8d: | 568 | 196 | 2.00E−49 | 32.6 | 24.5 | Transport |
| | | | | | | | protein |
| 4 | 1nqh: | 543 | 191 | 3.00E−48 | 32.2 | 24.1 | Transport |
| | | | | | | | protein |
| 5 | 2gsk: | 578 | 191 | 5.00E−48 | 30.6 | 26.3 | Signaling |
| | | | | | | | protein/membrane |
| | | | | | | | protein |
| 6 | 1nqe: | 488 | 189 | 2.00E−47 | 34.4 | 23 | Transport |
| | | | | | | | protein |
| 7 | 1ujw: | 507 | 188 | 4.00E−47 | 32 | 26.6 | Transport |
| | | | | | | | protein/hydro- |
| | | | | | | | lase |
| 8 | 3m8b: | 496 | 187 | 5.00E−47 | 34.3 | 24.6 | Transport |
| | | | | | | | protein |
| 9 | 1po3: | 588 | 181 | 3.00E−45 | 30.8 | 24.3 | Membrane |
| | | | | | | | protein |
| 10 | 2vqi: | 556 | 177 | 5.00E−44 | 30.9 | 24.5 | Transport |
| 11 | 2guf: | 474 | 177 | 5.00E−44 | 34.2 | 24.5 | Transport |
| | | | | | | | protein |
| 12 | 1nqg: | 605 | 177 | 7.00E−44 | 29.9 | 28.4 | Transport |
| | | | | | | | protein |
| 13 | 2ysu: | 530 | 175 | 3.00E−43 | 32.1 | 30.8 | Transport |
| | | | | | | | protein/hydro- |
| | | | | | | | lase |
| 14 | 2iah: | 454 | 175 | 3.00E−43 | 34.4 | 25.1 | Membrane |
| | | | | | | | protein |
| 15 | 3efm: | 466 | 169 | 1.00E−41 | 33.9 | 23.8 | Membrane |
| | | | | | | | protein |
| 16 | 1kmo: | 597 | 168 | 3.00E−41 | 29.8 | 27.5 | Membrane |
| | | | | | | | protein |
| 17 | 3fhh: | 603 | 167 | 7.00E−41 | 29.7 | 24 | Membrane |
| | | | | | | | protein |
| 18 | 1po3: | 563 | 167 | 7.00E−41 | 30.4 | 23.1 | Membrane |
| | | | | | | | protein |
| 19 | 1kmp: | 526 | 166 | 1.00E−40 | 32.9 | 24.9 | Membrane |
| | | | | | | | protein |
| 20 | 2b5m: | 706 | 165 | 2.00E−40 | 26.9 | 23.2 | DNA binding |
| | | | | | | | protein/ |
| | | | | | | | protein |
| | | | | | | | binding |

Mif1 Requires the N and C Terminus for Loading into the MACs Complex and the Entire Mif1 Protein is Required for the Induction of Metamorphosis.

To begin to understand how the Mif1 protein is loaded into the MACs complex, the protein was knocked out in 200 amino acid pieces across the 943 total amino acids and observed its ability to fill the inner lumen of the protein complex (FIG. 2, A). Previously we have shown that the 605 protein is required for Mif1 filling of the inner tube complex³⁷. Using cryo-electron microscopy we observed the ratio of filled versus empty tubes after growing these knockout strains and extracted MACs. Our results show the N-200aa and C-200aa portions of the protein complex were required for filling the protein complex (FIG. 2, B). Interestingly, both portions of the protein were predicted to contain globular alpha-helical domains and long (approx. 20aa) portions of unfolded residues. We predict that these long-unfolded portions of the protein contain signal sequences necessary for the loading of Mif1 into the inner tube complex⁴². These data give insight into required portions of the protein which are required for proper protein loading.

To further understand the portions of the protein required for function the Δ200aa knockouts were tested for metamorphosis. Adding MACs containing 200aa knockouts to Hydroides our results showed that the entire protein has portions which are required for metamorphosis and none of the 200aa knockouts proved to be functional. This is predictable as the complexity of the MACs complex likely limits the modifications of the effector proteins in both the loading, ejection into the host cell, and the function once it enters the host.

FIG. 2: The N and C-term domains are required for protein loading into the MACs inner tube complex. (A) Two hundred amino acid residues were systematically removed from Mif1 in order to determine their role in Mif1 effector loading. (B) The deletion mutants were then subject to cryo-electron microscopy to assess the filled versus empty status of the various deletions. These were plotted as a percentage of filled vs unfilled MACs structures (n=x). (C-G) Representative images from each knockout to determine the filled versus empty status. (D) Metamorphosis assays of extracted MACs complexes with the various mutants were tested and assessed for their ability to induce metamorphosis (the average of 3 biological replicates was plotted with the average of 4 technical replicates each).

Association of the 605 Protein with the Linker Regions Between Domains Suggest Stabilization of Unfolded Residues is Required for Loading into the MACs Complex

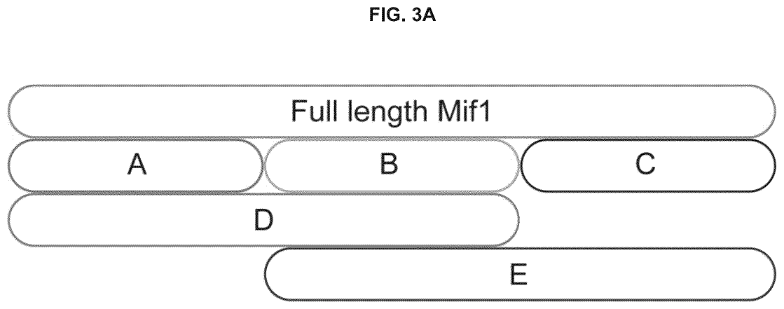

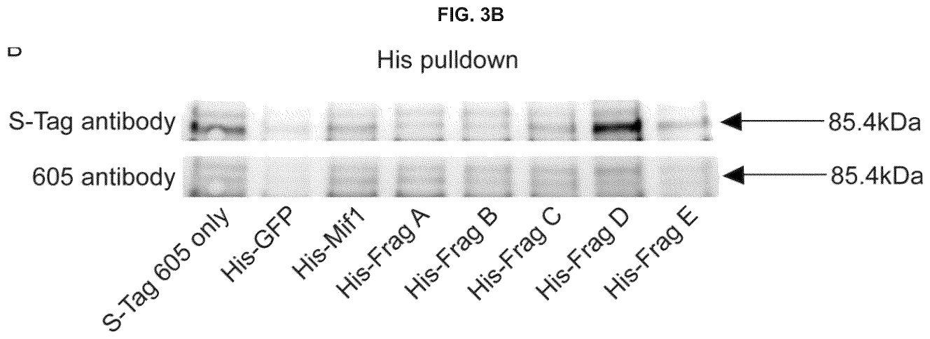

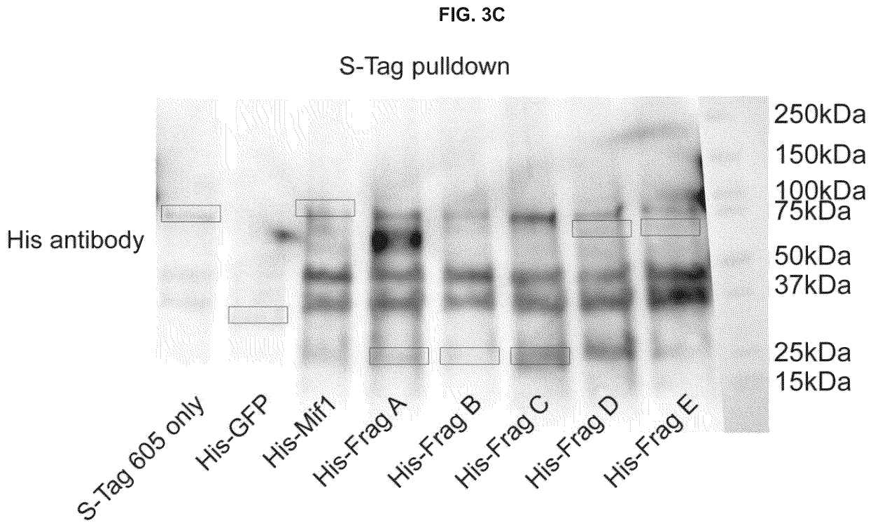

To understand the interaction that takes place between Mif1 and the loading chaperone protein 605 the two proteins were co-expressed within an expression host E. coli BL21 each with a different protein tag (S-tag 605 and His×6-Mif1). We have previously shown these two proteins interact⁴³ but did not know how where 605 binds to Mif1. To determine the binding interaction portion of Mif1 and 605 we broke the protein down into thirds (fragment A, B, and C) with two larger portions covering two thirds of the protein (fragment D and E) overlapping the smaller thirds in case the intersections are required for interaction (FIG. 3, A). The reciprocal pulldowns show that the 605 protein tightly binds to the D fragment of Mif1 which includes fragment A and B but spans the junction across them. This result is interesting because this junction falls at the 333aa which is after the portion of the protein that is required for loading. Our current hypothesis is the large unfolded connected region which exits between the 1-200aa alpha-helical region and the 200-760aa beta-pleated sheet region makes the protein wobbly. This wobble effects the ability for the protein to tightly pack into the small inner diameter of the MACs tube and therefore must be stabilized. By stabilizing the wobble portion of the protein, the protein can linearize and successfully pack into the MACs.

FIG. 3. Mif1 amino acid residues are required for binding with the MACs loading protein 1 (Mlp1). (a) Western blot showing the presence of Mlp1 tagged with a S-tag. Ni²⁺ agarose pull-down using Mif1 or Mif1 fragments was washed of unbound protein and the resultant preparation was blotted for the presence of Mlp1. Total lysate was used for comparison of pull-down protein versus total expressed protein. (b and c) The reciprocal S-tag was also used as bait and the Mif1 or Mif1 fragments were blotted by 6×His tag antibody.

### Mif1 Contains Two Toxin Domains Located at the N and C-Term of the Protein.

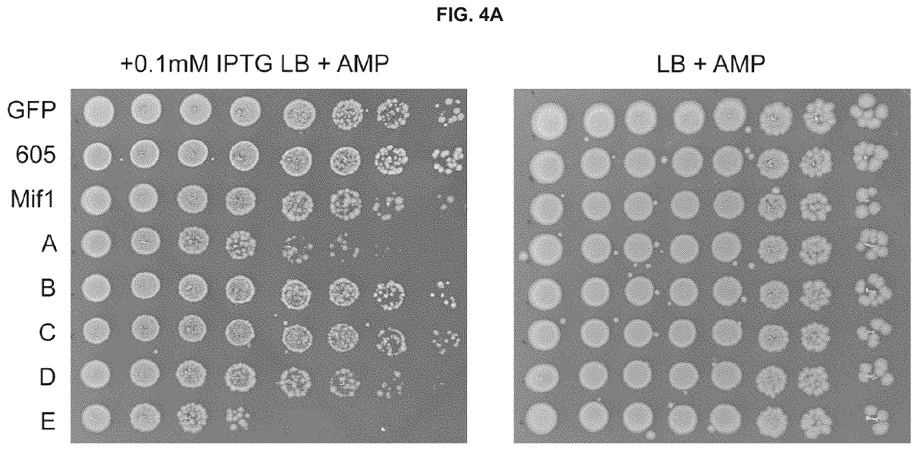

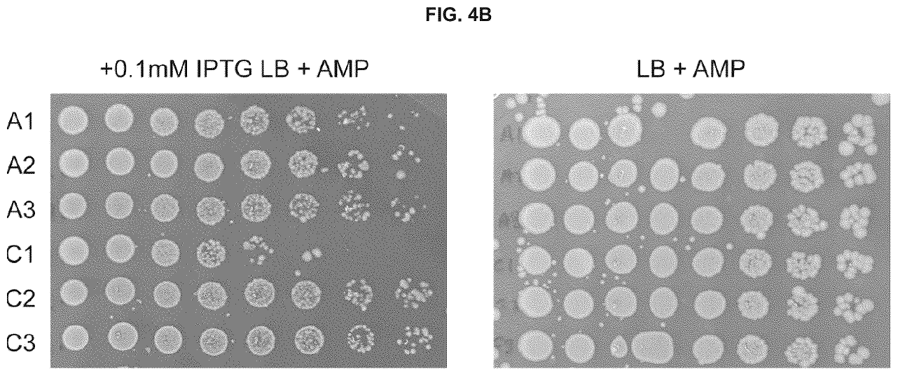

Besides the successful loading of the Mif1 protein into the MACs complex the protein also needs to perform some function once delivered to the Hydroides host. We noticed that during expression of the Mif1 fragments that certain portions of the Mif1 protein appeared to contain a toxin domain and were highly toxic to the E. coli. To assess the toxicity of Mif1, we expressed recombinant Mif1 in E. coli BL21 PlysE from the IPTG inducible T7 promoter. The A-E fragment regions of the protein were cloned and expressed. The cells were plated on a media containing 0.1 mM IPTG to drive expression of the fragments and serial diluted to determine death after expression. Our results show that the E and A fragment but not the full length protein lead to increased cell death (FIG. 4, A-B). These results suggest that protein may contain a toxin anti-toxin domain within the protein itself. This gives us insight into the folds that are predicted in each of these domains with the A fragment containing the lipase chaperone domain and the E fragment contain certain toxin domains. It is possible that the N and C termini of the protein interact during expression and prevent cell death from occurring, however, when one of the two termini are missing the protein becomes toxic.

After identifying the toxic regions located in the N and C terminus of the protein, we further broke those pieces into 3 more overlapping proteins A1-A3 and C1-C3. We Identified that the portion of the protein in the N-term likely requires more than the 150aa acid pieces that we broke the protein down into and may require up to the entire 338aa piece of the N-terminus. However, for The C-terminus we were able to identify the 150 aa C1 fragment as the portion of the protein found in the C-term responsible for its toxin phenotype.

We further confirmed these findings by looking for cell death via propidium iodide staining. These same E. coli constructs were expressed and then stained with the membrane impermeable nuclear stain propidium iodide. This will identify cells which have died and/or have compromised cellular membranes. Our results confirm the death assays performed by serial dilution on the IPTG plates.

### Mif1 Binds to Glycerophosphoinositol Membrane Lipids

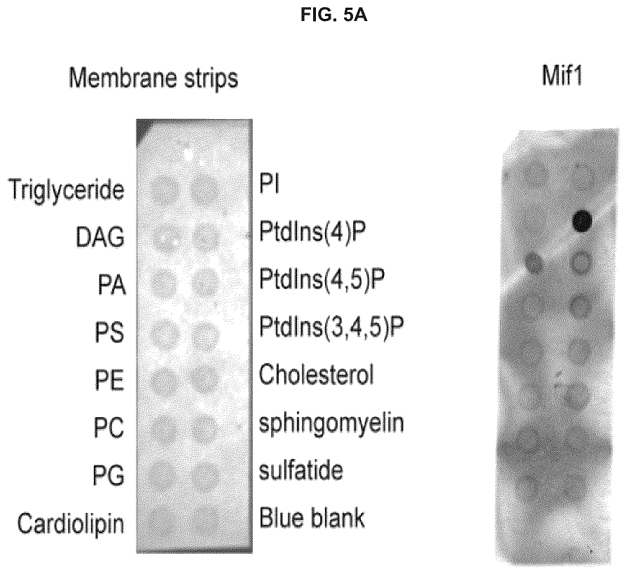

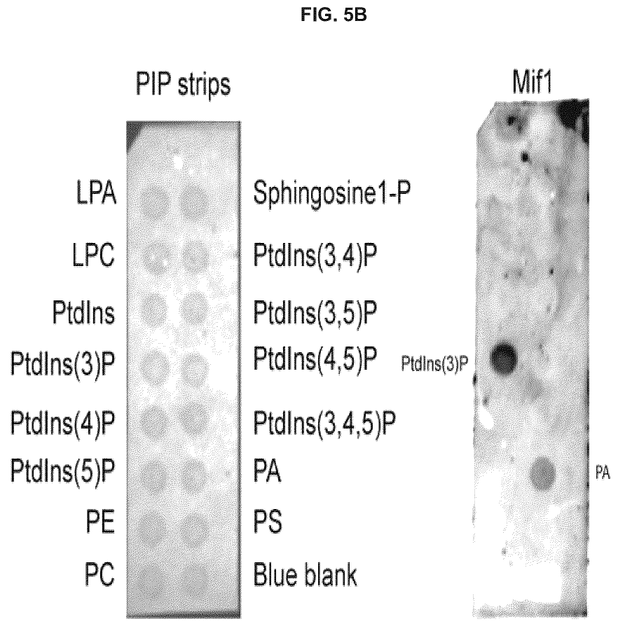

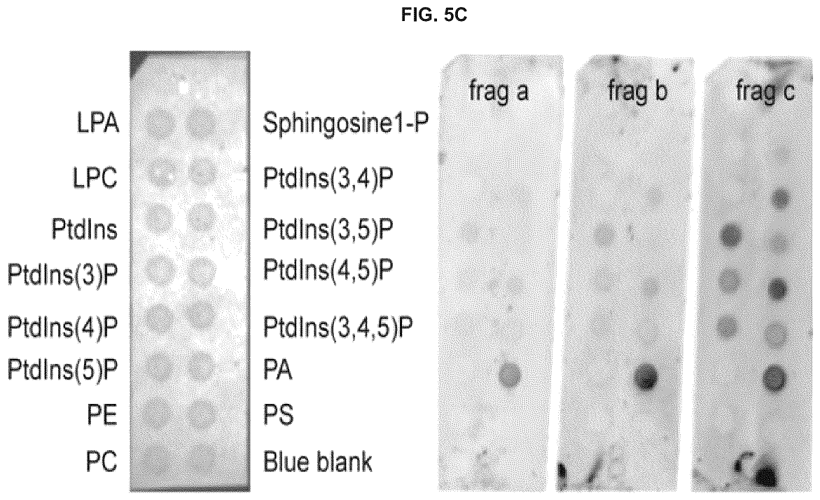

After identifying the disruption of the cellular membrane via propidium iodide staining, our next aim was to determine whether Mif1 was capable of binding to membrane lipids. Since Mif1 usually acts in the eukaryotic membranes of the Hydroides we screened the known membrane components which exist in eukaryotes. This was performed using membranes spotted with a range of membrane phospholipids and performed a far western blot to identify specific membrane binding interactions. If the protein successfully binds to the membranes, we can determine the relative association and preference based on spot intensity. Two commercially available membrane strips were used to assess binding, the first membrane strip showed that Mif1 was capable of binding to Phosphatidylinositol-4-phosphate (Ptdins(4)P) with high affinity and to a lesser degree Phosphatidic acid (PA) (FIG. 5, A). The second membrane with a variety of phosphatidylinositol isomers, showed Mif1 has high affinity for Ptdins(3,5)P over Ptdins(3)P or Ptdins(4)P (FIG. 5, B). Finally, To determine the binding region of Mif1 for the various phospholipids, the first, middle, and last third of the protein (fragment A, B, and C) were tested for binding. Our results show that interestingly all fragments were able to associate with phosphatidyl serine (PS), which was not seen with the full protein, it is unclear why the fragments but not the full protein was able to associate with this membrane lipid. Fragments A and B were only shown to bind to PS, however, fragment C was able to bind to the membrane lipids, PS, Ptdins(4,5)P, Ptdins(3)P, Ptdin(3,4)P, Ptdins(4)P and Ptdins(5)P in that order of preference (figure x, C). Our results showed that full length mif1 binds preferentially binds Ptdins(3,5)P>Ptdins(3)P>Ptdins(4)P>Ptdins(5)P>PA, in that order of preference.

FIG. 5. Mif1 binds membrane lipids and possesses lipase activity. (A) Lipid spotted membrane with various membrane lipids. (B) Far western using purified Mif1 protein and Mif1 specific antibody shows binding to both PI3P and PA. (C) Lipid cleavage assay with purified Mif1 protein or chaperone (12605) protein, incubated for 1 hour with decanoic acid-PNPP substrate. Cleavage and PnPP (4-nitrophenyl phosphate) release occurs if acyl-ester linkage is hydrolyzed. (D) PLD specific lipid cleavage assay with phosphatidylcholine substrate to assess enzymatic cleavage site of lipases by presence of choline release. Data are represented as the mean±SD of n=12 technical replicates across three independent biological replicates. Significance is indicated as a comparison between the two conditions indicated by the line above (***p<0.0001; ***p<0.001).

Association of bacterial proteins with inositol phospholipids is unique due to most bacteria do not make inositol glycolipids. These data suggest a role for this protein specifically in eukaryotes and potentially targeted to specific compartments within the eukaryotic host. The finding of the toxin domain within the E and C1 fragments illuminate the possibility of an enzymatic activity which resides within these fragments and acts non-specifically. The ability to bind to specific lipids and have toxin activity need not be mutually exclusive. The association with membrane lipids and toxin activity suggest a role for this protein as a phospholipase. Since Mif1 is associated with inositol phospholipids which make up a tiny fraction of the total membrane lipids it is possible that Mif1 is a lipase that acts as a signaling molecule producing lipid second messengers. As mentioned previously DAG is upregulated in Hydroides only when Mif1 is present. However, an equally likely alternative possibility exits, it is possible that Mif1 associates with the membrane and creates a membrane ion gradient via pore formation. This could lead to the downstream activation of a membrane phospholipid which cleaves membrane lipids and produces DAG. To determine whether Mif1 may act as a lipase we performed lipase assays with Mif1.

### Mif1 Possesses Esterase Activity and is Able to Cleave PLA1 and PLD Phospholipid Type Cleavages

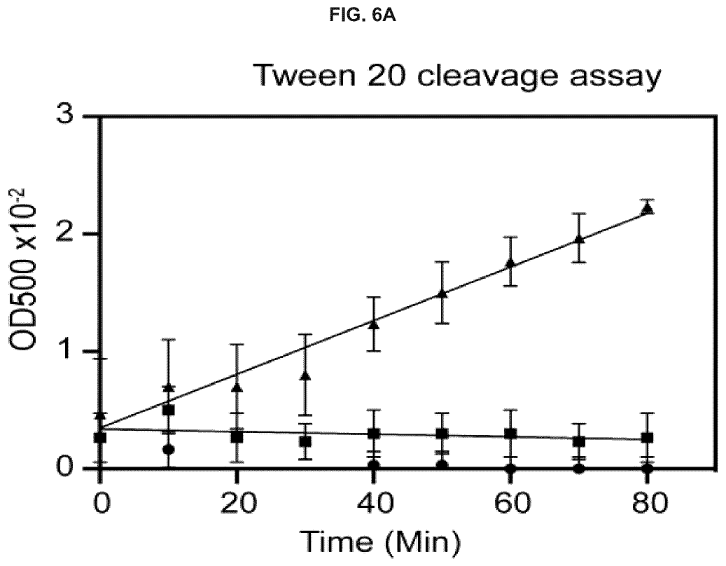

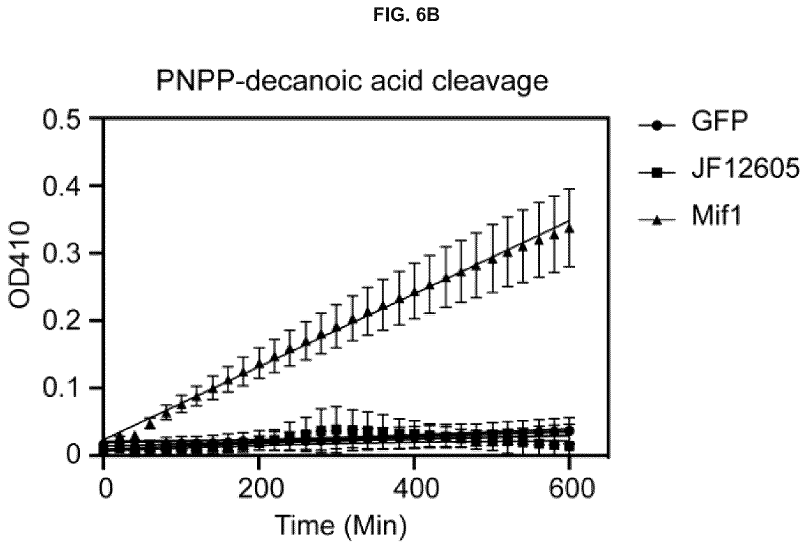

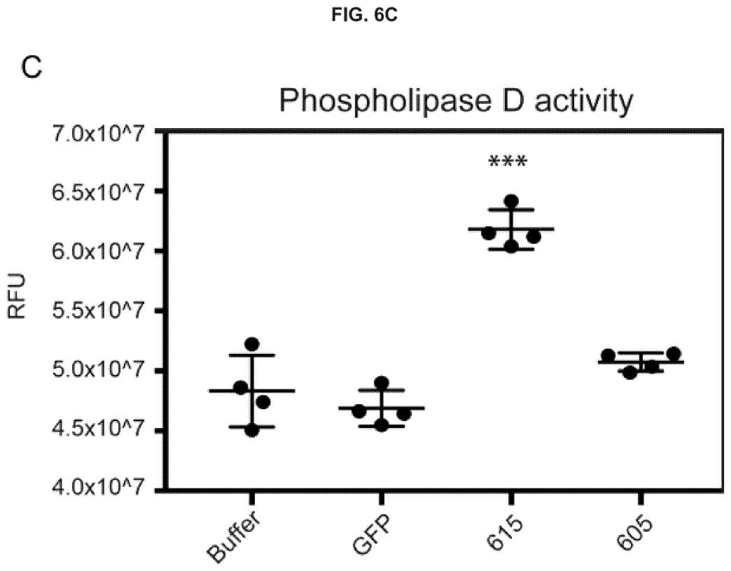

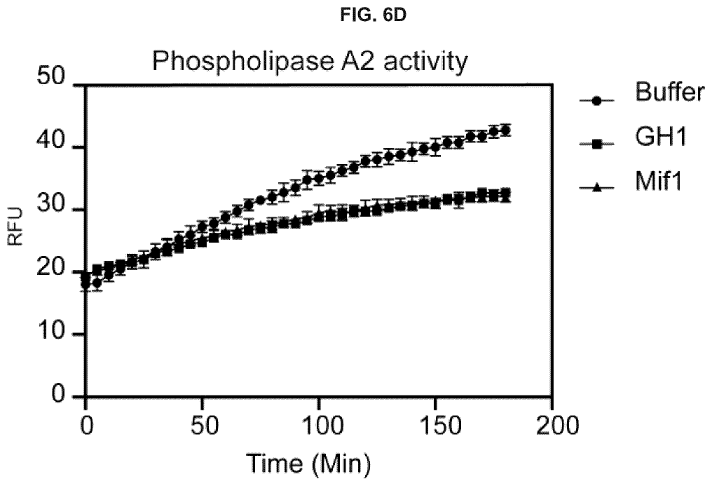

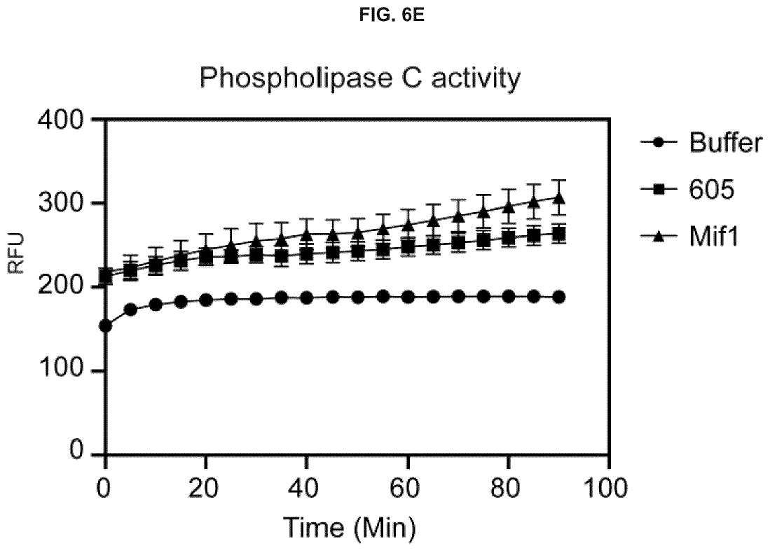

After determining Mif1's association with membrane lipids, we wanted to test for enzymatic activity. Since membrane binding is a feature that all lipases possess. We then tested for enzymatic activity of the protein by looking at the proteins ability to cleave acyl-ester linkages which is a common function across most lipases. We determined through cleavage of a synthetic lipid decanoic acid-PNPP fusion and through the cleavage of TWEEN-20™, Mif1 possesses the ability to cleave acyl ester linkages (FIGS. 6, A and B).

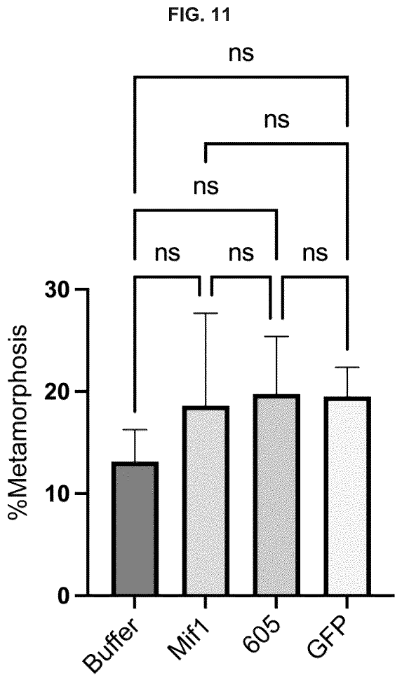

These results prompted us to look further into the specific activity and cleavage products of the protein/Since there are many different potential enzymatic cleavage sites of phospholipases, we tested all the common cleavage types. Mif1 was assayed for specificity to Phospholipase A2 (PLA2), Phospholipase C (PLC), and Phospholipase D (PLD) activity. We saw no activity from the PLA2 specific, or PLC specific assays, however we did see some increased activity in the PLD assay (Figure x, D). PLA1 activity has previously been measured using the tween-20 and PNPP-decanoic acid assays which are a form of PLA1 type cleavages. This result further validates our binding experiments which show that MIF1 can strongly bind to PIPs and weakly binds to PA, which would be the resultant product from a PLD type cleavage. These data suggest a role for Mif1 as a phospholipase in the membrane and a potential activator of lipid second messengers. It is unclear whether the PLD activity or the PLA1 activity are required for metamorphosis by Mif1 and the products produced (decanoic acid and phosphatidic acid) by either of these activities were not successful in stimulating metamorphosis (see FIG. 11).

FIG. 6. Mif1 possesses lipase activity. (A) Lipid cleavage assay with purified Mif1 protein or chaperone (12605) protein, or a GFP control protein incubated with Tween-20 in the presence of Ca²⁺. (B) Purified proteins incubated for 1 hour with decanoic acid-PNPP substrate. Cleavage and PnPP (4-nitrophenyl phosphate) release occurs if acyl-ester linkage is hydrolyzed. (C) PLD specific lipid cleavage assay with phosphatidylcholine substrate to assess enzymatic cleavage site of lipases by presence of choline release. (D) Phospholipase A2 specific cleavage assay with Mif1, Buffer, or a control protein GH1. (E) Phospholipase C specific cleavage assay with Buffer, Mif1 or 605 control protein. Data are represented as the mean±SD of n=12 technical replicates across three independent biological replicates. Significance is indicated as a comparison between the two conditions indicated by the line above (***p<0.0001; ***p<0.001).

### Mif1 Three Identified Domains Work Cooperatively to Cleave Lipids

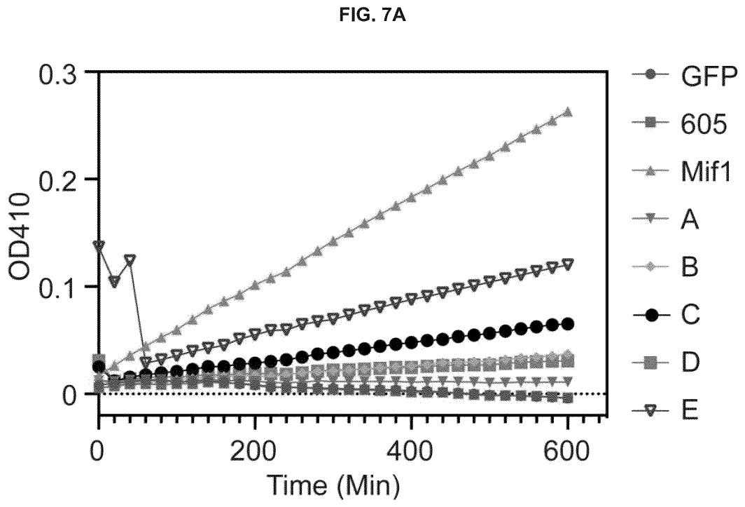

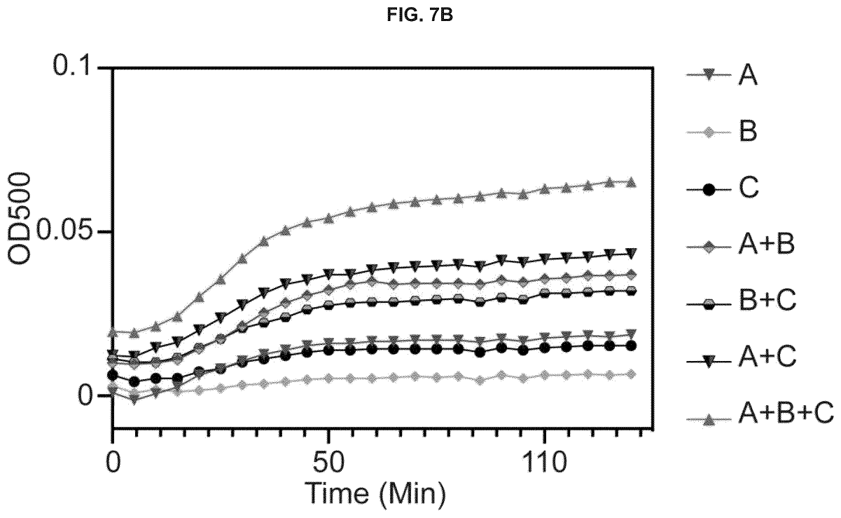

To determine the portion of the protein which contains the lipase activity we analyzed each of the Mif1 expression fragments for lipase activity via PNPP-decanoic acid cleavage assy. Our results show that the E and C fragment which contain the C-terminal portion of the protein possessed the most activity (FIG. 7, b). However, we did identify some activity associated with just the A fragment of the protein. To better understand how each of the fragments contribute to the Mif1 lipase activity we combinatorial combined these fragments together. Each of the fragments were expressed in a separate E. coli and purified. After purification the protein concentrations were normalized and added together such that the total protein concentration was kept constant. After combining the fragments together, the lipase assay was repeated to determine how the protein works together. Our findings show that the Mif1 protein acts cooperatively with each of the fragments contributing to the lipase function. Where the A+B+C fragment retained the most activity followed by the A+C fragment and then by the A+B and B+C, finally the C and A fragment alone possessed some activity while the B fragment alone contained no lipase activity (FIG. 7, C).

These data suggest that the Mif1 protein requires all the protein for wild-type activity and no isolated domain exists which contains the lipase activity. These data corroborate the findings showing any of the 200aa deletions in the protein resulted in a loss of function for the metamorphic activity of this protein. The complexity of these findings obscures the potential targeted KO of a domain or single residue which could be responsible for the activity. Likely many portions of the protein contribute to these functions.

Many Type 6 Secretion System effectors exert their effect on target cells by lipase activity. Many of these lipases are cytotoxic lipases that are used as toxin proteins and delivered to induce cell death in the target cells usually bacteria antagonists. However, there are examples of lipases which produce very specific signaling to occur within the host cell. One example is the MARTX toxins from Vibrio cholerae, these are large proteins which self-cleave into smaller functional proteins. The MARTX toxin has been shown to bind PIPs and through PLA1 cleavage inhibit endosomal trafficking and autophagy⁴⁴. These proteins are not generic toxics like Phosphatidylcholine specific PLDs which completely degrade the major component of the cellular membrane, but instead act on low abundance lipids and specific signaling pathways within the cell. MIF1 likely acts through a different signal cascade than the MARTX toxin since is not toxic to Hydroides larvae, however, since the lipids Mif1 targets are found on endosomes and lysosomes PI(3,5)P, PI(4)P, and PI(3)P, the target lipids and subcellular localization may be shared. The protein may also be cleaved and facilitate its own activity or act as an anti-toxin within the host cell. Our data illuminates the complexity of these large effector toxins and their potential role in multiple different signaling processes.

### CONCLUSION

Our finding show that the Mif1 protein requires the N and C terminus for loading into the MACS complex. The 605 chaperone previously characterized to facilitate loading binds to an unfolded portion of the protein stabilizing what appears to be a wobble region. Systematic knock out analysis of 200 bp within the Mif1 protein shows that the entire protein is required for function as a metamorphosis inducing protein. Through expression of the protein and Far-western blot analysis on membranes impregnated with various eukaryotic phospholipids, we identify that Mif1 has the ability to bind to inositol phospholipids and phosphatidic acid. Through fragment expression breaking the Mif1 protein into 5 different fragments we characterize the protein has ability to bind Lipids through its C-terminus. By further analysis of the fragments, we identified a toxin domain located in the C-terminus of the protein, and the 1-200aa N-terminus as well. These fragments were further characterized to possess lipase activity and likely to cleave both PLA1 and PLD type lipid linkages. When combining the fragments, the protein appears to cooperatively increase lipase activity suggesting interactions between the entire protein are required for wild-type levels of activity. Our findings demonstrate the role of a bacterial lipase effector and its functional abilities, which furthers our understanding the complex nature of bacteria-animal signaling interactions that take place.

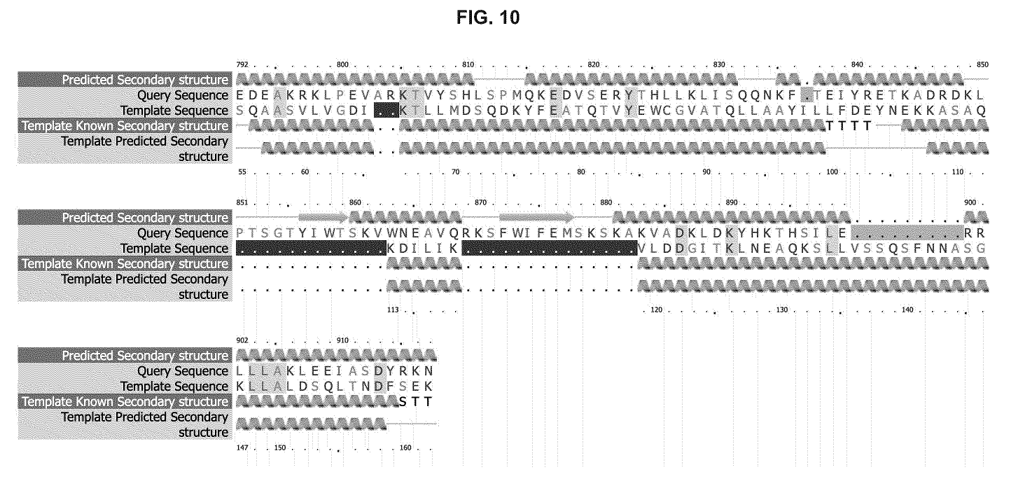

FIG. 10 schematically illustrates the alignment of Mif1 and E. coli hemolysin E pore forming toxin via PHYRE2™ (Protein Homology/AnalogY Recognition Engine) (Creative Commons Attribution-2.0);

SEQ ID NO: 6 illustrates the query sequence:
EDEAKRKLPEVARKTVYSHLSPMQKEDVSERYTHLLKLISQQNKFTPTS
GTYIWTSKVWNEAVQRKSFWIFEMSKSKAKVADKLDKYHKTHSILLLAK
LEEIASDYRKN
SEQ ID NO: 7 illustrates the template sequence:
SQAASVLVGDIKTLLMDSQDKYFEATQTVYEWCGVATQLLAYILKDILI
KVLDDGITKLNEAQKSKLLALDSQLTNDFSEKs

### REFERENCES

1. Shikuma, N. J. Bacteria-Stimulated Metamorphosis: an Ocean of Insights from Investigating a Transient Host-Microbe Interaction. mSystems 6, 1-5 (2021).
2. Jandhyala, S. M. et al. Role of the normal gut microbiota. World J. Gastroenterol. 21, 8787-8803 (2015).
3. Aschtgen, M.-S., et al. Vibrio fischeri-derived outer membrane vesicles trigger host development. Cell. Microbiol. 18, 488-499 (2016).
4. Hill, J. H., et al. A conserved bacterial protein induces pancreatic beta cell expansion during zebrafish development. Elife 5, 1-18 (2016).
5. Zobell, C. E. & Allen, E. C. The Significance of Marine Bacteria in the Fouling of Submerged Surfaces. J. Bacteriol. 29, 239-51 (1934).
6. Cavalcanti, G., et al. The Influence of Bacteria on Animal Metamorphosis. Annu. Rev. Microbiol. 74, in press (2020).
7. Hadfield, M. G. Why and how marine-invertebrate larvae metamorphose so fast. Semin. Cell Dev. Biol. 11, 437-443 (2000).
8. Wang, Y. & Ruby, E. G. The roles of NO in microbial symbioses. Cell. Microbiol. 13, 518-526 (2011).
9. Chambon, J.-P., et al. ERK- and JNK-signalling regulate gene networks that stimulate metamorphosis and apoptosis in tail tissues of ascidian tadpoles. Development 134, 1203-1219 (2007).
10. Amador-Cano, G., et al. Role of Protein Kinase C, G-Protein Coupled Receptors, and Calcium Flux During Metamorphosis of the Sea Urchin Strongylocentrotus purpuratus. Biol. Bull. 210, 121-131 (2006).
11. Wang, H. & Qian, P.-Y. Involvement of a novel p38 mitogen-activated protein kinase in larval metamorphosis of the polychaete Hydroides elegans (Haswell). J. Exp. Zool. B. Mol. Dev. Evol. 314, 390-402 (2010).
12. McCauley, D. W. Serotonin plays an early role in the metamorphosis of the hydrozoan Phialidium gregarium. Dev. Biol. 190, 229-240 (1997).
13. Moniri, N. H. et al. Role of PKA and PKC in histamine H1 receptor-mediated activation of catecholamine neurotransmitter synthesis. Neurosci. Lett. 407, 249-253 (2006).
14. Henningi, G., et al. Metamorphic processes in the soft corals Heteroxenia fuscescens and Xenia umbellata: The effect of protein kinase c activators and inhibitors. Invertebr. Reprod. Dev. 34, 35-45 (1998).
15. Leitz, T. Biochemical and cytological bases of metamorphosis in Hydractinia echinata. Mar. Biol. Int. J. Life Ocean. Coast. Waters 116, 559-564 (1993).
16. Unabia, C. R. C. et al. Role of bacteria in larval settlement and metamorphosis of the polychaete Hydroides elegans. Mar. Biol. 133, 55-64 (1999).
17. Biggers, W. J. & Laufer, H. Settlement and Metamorphosis of Capitella Larvae Induced by Juvenile Hormone-Active Compounds Is Mediated by Protein Kinase C and Ion Channels. Biol. Bull. 196, 187-198 (1999).
18. Freeman, G. et al. Cellular and intracellular pathways mediating the metamorphic stimulus in hydrozoan planulae. Roux's Arch. Dev. Biol. 199, 63-79 (1990).
19. Leitz, T. & Klingmann, G. Metamorphosis inHydractinia: Studies with activators and inhibitors aiming at protein kinase C and potassium channels. Roux's Arch. Dev. Biol. 199, 107-113 (1990).
20. Yamamoto, H., et al. Protein Kinase C (PKC) Signal Transduction System Involved in Larval Metamorphosis of the Barnacle, Balanus amphitrite. Zoological Science 12, 391-396 (1995).
21. Freckelton, M. L. et al. Bacterial lipopolysaccharide induces settlement and metamorphosis in a marine larva. bioRxiv (2019). doi:doi: http://dx.doi.org/10.1101/851519
22. Guo, H., et al. Two Distinct Bacterial Biofilm Components Trigger Metamorphosis in the Colonial Hydrozoan Hydractinia echinata. MBio 12, (2021).
23. Geller, A. M. et al. The extracellular contractile injection system is enriched in environmental microbes and associates with numerous toxins. Nat. Commun. 2021 121 12, 1-15 (2021).
24. Fraser, A. D., Plattner, M. & Leiman, P. G. Energetics of Sheath Contraction in Contractile Injection Systems. Biophys. J. 112, 334a (2017).
25. Shikuma, N. J. et al. Marine tubeworm metamorphosis induced by arrays of bacterial phage tail-like structures. Science (80-). 343, 529-33 (2014).
26. Flaugnatti, N. et al. A phospholipase A 1 antibacterial Type VI secretion effector interacts directly with the C-terminal domain of the VgrG spike protein for delivery. doi:10.1111/mmi.13292
27. Sana, T. G. et al. Salmonella TYPHIMURIUM utilizes a T6SS-mediated antibacterial weapon to establish in the host gut. Proc. Natl. Acad. Sci. U.S.A 113, E5044-51 (2016).
28. Aubert, D. F. et al. A Burkholderia Type VI Effector Deamidates Rho GTPases to Activate the Pyrin Inflammasome and Trigger Inflammation. Cell Host Microbe 19, 664-674 (2016).
29. Ma, L. S., et al. Agrobacterium tumefaciens deploys a superfamily of type VI secretion DNase effectors as weapons for interbacterial competition in planta. Cell Host Microbe 16, 94-104 (2014).
30. Russell, A. B. et al. Diverse type VI secretion phospholipases are functionally plastic antibacterial effectors. Nature 496, 508-512 (2013).
31. Chatzidaki-Livanis, M., et al. Bacteroides fragilis type VI secretion systems use novel effector and immunity proteins to antagonize human gut Bacteroidales species. Proc. Natl. Acad. Sci. 113, 3627-3632 (2016).
32. A., S. H. et al. Disruption of lipid homeostasis in the Gram-negative cell envelope activates a novel cell death pathway. Proc. Natl. Acad. Sci. 113, E1565-E1574 (2016).
33. Cummings, B. S., et al. Phospholipase A(2)s in cell injury and death. J. Pharmacol. Exp. Ther. 294, 793-799 (2000).
34. Bender, J. et al. Lipases as Pathogenicity Factors of Bacterial Pathogens of Humans BT—Handbook of Hydrocarbon and Lipid Microbiology. in (ed. Timmis, K. N.) 3241-3258 (Springer Berlin Heidelberg, 2010).
35. Suh, P. G. et al. Multiple roles of phosphoinositide-specific phospholipase C isozymes. J. Biochem. Mol. Biol. 41, 415-434 (2008).
36. Rocchi, I. et al. A Bacterial Phage Tail-like Structure Kills Eukaryotic Cells by Injecting a Nuclease Effector. Cell Rep. 28, 295-301.e4 (2019).
37. Nicholas J. Shikuma, Beyhan, S., Pilhofer, M. & Ericson, C. Protein and peptide delivery systems and methods for making and using them. (2019).
38. Unabia, C. R. C. et al. Role of bacteria in larval settlement and metamorphosis of the polychaete Hydroides elegans. Mar. Biol. 133, 55-64 (1999).
39. Malter, K. E. et al. Diacylglycerol, PKC and MAPK Signaling Initiate Tubeworm Metamorphosis in Response to Bacteria. Dev. Biol. (2022). doi:https://doi.org/10.1016/j.ydbio.2022.04.009
40. Welte, W., Nestel, U., Wacker, T. & Diederichs, K. Structure and function of the porin channel. Kidney Int. 48, 930-940 (1995).
41. Carpizo-Ituarte, E. et al. Stimulation of metamorphosis in the polychaete Hydroides elegans Haswell (Serpulidae). Biol. Bull. 194, 14-24 (1998).
42. Quentin, D. et al. Mechanism of loading and translocation of type VI secretion system effector Tse6. Nat. Microbiol. 3, 1142-1152 (2018).
43. Rocchi, I. et al. A Bacterial Phage Tail-like Structure Kills Eukaryotic Cells by Injecting a Nuclease Effector. Cell Rep. 28, 295-301.e4 (2019).
44. Agarwal, S. et al. Autophagy and endosomal trafficking inhibition by Vibrio cholerae MARTX toxin phosphatidylinositol-3-phosphate-specific phospholipase A1 activity. Nat. Commun. 6, 8745 (2015).

A number of embodiments of the invention have been described. Nevertheless, it will be understood that various modifications may be made without departing from the spirit and scope of the invention. Accordingly, other embodiments are within the scope of the following claims.

1: A chimeric product of manufacture for delivering a proteinaceous cargo, or a heterologous protein or peptide, or a compound, into a cell, comprising:
(a) a recombinant bacterial Contractile Injection System (CIS) or a Metamorphosis Associated Contractile structure (MAC) formed or configured to comprise a tube having an inner core,
(b) a Metamorphosis-Inducing Factor 1 (Mif1) protein positioned in the inner core of the tube of the CIS or MAC,
(c) a chaperone 605 protein non-covalently associated with or covalently associated with or linked to the Mif1 protein positioned in the inner core of the tube of the CIS or MAC, and
(d) a proteinaceous cargo, or a heterologous protein or peptide, or compound, non-covalently associated or covalently associated or linked to the Mif1.

2: A liposome or lipid-comprising nanoparticle comprising, or incorporating or expressing on its outer surface, a chimeric product of manufacture of claim 1.

3: A protoplast or a spheroplast comprising, or incorporating or expressing on its outer surface, a chimeric product of manufacture of claim 1.

4: A cell comprising, or expressing on its extracellular surface, a chimeric product of manufacture of claim 1.

5: A method for delivering a proteinaceous cargo, or a protein or a peptide, or a compound, to a cell, optionally to a eukaryotic, mammalian or human cell, or to a plant cell, or to an individual in need thereof, comprising contacting the cell with:
a chimeric product of manufacture of claim 1,
under conditions wherein the proteinaceous cargo, or the protein or peptide, or the compound, is delivered into the cell.

6: The method of claim 5, wherein the proteinaceous cargo, or the protein or peptide, comprises or is an antibody or an enzyme or an active biological agent.

7: The method of claim 5, wherein the contacting of the formulation or composition with the cell eukaryotic cell is in vitro, ex vivo, or in vivo.

8: The method of claim 1, wherein the eukaryotic cell is a mammalian, human or an animal cell.

9: A pharmaceutical composition comprising:
a chimeric product of manufacture of claim 1.

10: A kit comprising:
a chimeric product of manufacture of claim 1,
wherein optionally the kit further comprises instructions for practicing a method of any of the preceding claims.

11-12: (canceled)

13: The chimeric product of manufacture of claim 1, wherein the proteinaceous cargo, the heterologous protein or peptide, or drug is chemically linked or electrostatically linked to the Mif1.

14: The chimeric product of manufacture of claim 1, wherein the compound is or comprises a small molecule, a lipid, a saccharide, a nucleic acid, a drug or a marker, optionally a detectable marker or a detectable moiety.

15: The chimeric product of manufacture of claim 1, wherein the proteinaceous cargo, the heterologous protein or peptide has enzymatic activity, optionally a lipase activity.

16: The chimeric product of manufacture of claim 13, wherein the proteinaceous cargo, the heterologous protein or peptide has binding activity, optionally heterologous protein or peptide comprises an antibody or antigen binding fragment.

17: The chimeric product of manufacture of claim 1, wherein the Mif1 protein is encoded by a nucleic acid sequence having at least about 85%, 86%, 87%, 88%, 89%, 90%, 91%, 92%, 93%, 94%, 95%, 96%, 97%, 98% or 100% sequence identity to SEQ ID NO:1, or between about 80% to 100% sequence identity to SEQ ID NO:1.

18: The chimeric product of manufacture of claim 1, wherein the Mif1 protein comprises a sequence having at least about 85%, 86%, 87%, 88%, 89%, 90%, 91%, 92%, 93%, 94%, 95%, 96%, 97%, 98% or 100% sequence identity to SEQ ID NO:2, or between about 80% to 100% sequence identity to SEQ ID NO:2.

19: The chimeric product of manufacture of claim 1, wherein the CIS or MAC proteins are encoded by a nucleic acid sequence having at least about 85%, 86%, 87%, 88%, 89%, 90%, 91%, 92%, 93%, 94%, 95%, 96%, 97%, 98% or 100% sequence identity to SEQ ID NO:5, or between about 80% to 100% sequence identity to SEQ ID NO:5.

20: The chimeric product of manufacture of claim 1, wherein the chaperone 605 protein is encoded by a nucleic acid sequence having at least about 85%, 86%, 87%, 88%, 89%, 90%, 91%, 92%, 93%, 94%, 95%, 96%, 97%, 98% or 100% sequence identity to SEQ ID NO:3, or between about 80% to 100% sequence identity to SEQ ID NO:3.

21: The chimeric product of manufacture of claim 1, wherein the chaperone 605 protein comprises a sequence having at least about 85%, 86%, 87%, 88%, 89%, 90%, 91%, 92%, 93%, 94%, 95%, 96%, 97%, 98% or 100% sequence identity to SEQ ID NO:4, or between about 80% to 100% sequence identity to SEQ ID NO:4.

22: The cell of claim 4, wherein the cell is a microbial cell or a eukaryotic cell, and optionally the microbial cell is a bacterial cell or a yeast cell.
